(19) United States
(12) Patent Application Publication
(10) Pub. No.: US 2026/0092259 A1
(43) Pub. Date: Apr. 2, 2026

(54) CELL ADHESION SYSTEM FOR INTERSPECIES CHIMERAS

(71) Applicant: THE BOARD OF REGENTS OF THE UNIVERSITY OF TEXAS SYSTEM, Austin, TX (US)

(72) Inventors: Jun Wu, Dallas, TX (US); Emily B. Ballard, Dallas, TX (US); Jia Huang, Dallas, TX (US)

(21) Appl. No.: 19/286,057

(22) Filed: Jul. 30, 2025

Related U.S. Application Data
(60) Provisional application No. 63/678,050, filed on Jul. 31, 2024.

(51) Int. Cl. C12N 5/071; C12N 5/074

(52) U.S. Cl. CPC C12N 5/0697; C12N 5/0696; C12N 2513/00; C12N 2517/02; C12N 2533/50

(57) ABSTRACT

Disclosures herein are directed to populations of cells comprising one or more cell of a first mammalian species and one or more cell of a second mammalian species, such as stem cells and embryos, wherein the cells are engineered to express an either an antigen-binding protein or an antigen, wherein each are expressed with a cell membrane signaling peptide and/or a cell membrane anchor protein. The inclusion of the cell membrane signaling peptide or a cell membrane anchor protein enhances cell adhesion, enabling improved formation of interspecies chimeras, for example the generation of a human and non-human interspecies chimera. Also provided herein are methods of manufacturing the population of cells as described herein. Also provided are methods of organogenesis to generate an organ, organoid, or tissue mass comprising an interspecies chimeric animal for organ transplant, and methods of treatment using the same.

### SEQUENCE LISTING

The instant application contains a Sequence Listing which has been submitted electronically in Extensible Markup Language (.xml) format and is hereby incorporated by reference in its entirety. Said XML copy, created on Dec. 15, 2025, is named “106546-852065-4402 SL ST26.xml” and is 643,072 bytes in size.

### FIELD OF THE INVENTION

The present disclosure relates to improvement of cell adhesion systems and their use in developing interspecies chimeras for organogenesis for organ transplant.

### BACKGROUND

Organ and tissue transplant can save lives, however according to the CDC on any given day in the United States (US), there are around 100,000 people on the active waiting list for organs, but only approximately 14,000 deceased organ donors (data from 2021), with each providing on average 3.5 organs. Living donors provide on average only around 6,000 organs per year. Additionally, in the US, around 3.3 million tissue grafts are distributed each year. About 2.5 million grafts are transplanted. Organ and graft rejection is also a common problem that can result in serious complications and even death.

The technique of generating interspecies chimeras using human pluripotent stem cells (hPSCs) is a promising in vivo platform to study human development and offers a potential source for growing personalized human tissue and donor organs in animals. Although robust chimerism can be achieved between closely related species, it is far more difficult to generate chimeras between evolutionarily distant species. The low chimerism of human cells in animals (e.g., mice and pigs) is presumably due to multiple xenogeneic barriers during early development, which include but are not limited to differences in developmental pace, incompatibility in cell adhesion molecules, and interspecies cell competition. Several strategies have been developed to improve human cell chimerism in animal embryos by genetically inhibiting human cell apoptosis. However, these strategies are not practical for future use in regenerative medicine as the modified genes and pathways are mostly oncogenic.

There is therefore a need in the field for methods to facilitate and improve establishment of mammalian interspecies chimeric cell populations for proliferation mammalian host animals.

### SUMMARY

Provided herein is a population of cells, comprising one or more cell of a first mammalian species and one or more cell of a second mammalian species, wherein the one or more cell of the first mammalian species comprises an exogenous nucleic acid encoding an antigen-binding protein that specifically binds to an antigen; and a cell membrane signaling peptide and/or a cell membrane anchor protein, wherein the antigen-binding protein is expressed at the cell surface of the one or more cell of the first mammalian species and specifically binds to an antigen at the cell surface of the one or more cell of the second mammalian species, and wherein the antigen is endogenous or exogenous to the one or more cell of a second mammalian species.

The cell membrane signaling peptide of the one or more cell of the first mammalian species may comprise at least one transmembrane domain; bind to a signal recognition protein (SRP); target the antigen-binding protein to the endoplasmic reticulum (ER) membrane; introduce a lipid anchor; and/or localize the antigen-binding protein to the cell membrane. The cell membrane signaling peptide of the one or more cell of a first mammalian species introduces a lipid anchor, optionally wherein the lipid anchor is glycosylphosphatidylinositol (GPI). The cell membrane signaling peptide may comprise an amino acid sequence selected from any one of SEQ ID NOS: 1-4 or 8, or an amino acid sequence having at least 80% identity thereto.

The cell membrane anchor protein of the one or more cell of the first mammalian species may comprise at least one transmembrane domain; localize the antigen-binding protein to cell surface of the cell of a first mammalian species; and/or is glycosylated or is not glycosylated. The cell membrane anchor protein of the one or more cell of the first mammalian species may comprise one or more transmembrane domain selected from a CD8 transmembrane domain, ICAM1 transmembrane domain, E-cadherin transmembrane domain, CLDN6 transmembrane domain, ITGB 1 transmembrane domain, ITGB2 transmembrane domain, JAM-B transmembrane domain, NCAM-1 transmembrane domain, MUC-4 transmembrane domain, PDGFRA transmembrane domain, or fragment thereof each. The cell membrane anchor protein of the one or more cell of the first mammalian species may selected from CD8, ICAM1, E-Cad, CLDN6, ITGB1, ITGB2, JAM-B, NCAM-1, MUC-4, PDGFRA, or a fragment thereof each. The cell membrane anchor protein of the one or more cell of the first mammalian species may comprise an amino acid sequence comprising the sequence set forth in any one of SEQ ID NO:9-13, or an amino acid sequence having at least 80% identity thereto.

The antigen-binding protein of the one or more cell of a first mammalian species may comprise an antibody or antigen-binding fragment thereof, optionally wherein the antigen-binding protein comprises a fragment variable domain (FV); a fragment antigen binding (Fab) domain; a Fab′; a (Fab′) 2; a half-IgG; a single-chain fragment variable domain (scFv); a diabody (di-scFv); a triabody (tri-scFv)′ a single chain Fab (scFab); a minibody; an scFv-Fc, or a nanobody.

The antigen-binding protein of the one or more cell of a first mammalian species may be a nanobody, optionally wherein the nanobody may be selected from an anti-GFP nanobody, further optionally wherein the anti-GFP nanobody may be selected from vhhGFP4, LaG-2, LaG-3, LaG-5, LaG-6, LaG-8, LaG-9, LaG-10, LaG-11, LaG-12, LaG-17, LaG-18, LaG-43, LaG-19, LaG-21, LaG-42, LaG-24, LaG-26, LaG-27, LaG-29, LaG-30, LaG-35, LaG-37, LaG-41, LaM-2, LaM-3, LaM-4, LaM-6, or LaM-8. The antigen-binding protein of the one or more cell of a first mammalian species may comprise the amino acid sequence set forth in any one of SEQ ID NOS: 9-36, or a sequence having at least 80% identity thereto.

The antigen-binding protein, cell membrane signaling peptide, and/or a cell membrane anchor protein of the cell of the first mammalian species may be connected with linker, optionally wherein the linker comprises the amino acid sequence set forth in any one of SEQ ID NOS: 5-7.

The antigen of the one or more cell of the second mammalian species may be endogenous to the second mammalian species, optionally may be endogenous to the first and second mammalian species.

The antigen of the one or more cell of the second mammalian species may be exogenous to the second mammalian species, optionally may be exogenous to the first and second mammalian species.

The one or more cell of the second mammalian species may comprise an exogenous nucleic acid encoding the exogenous antigen; and a cell membrane signaling peptide and/or a cell membrane anchor protein. The exogenous antigen may be selected from mCherry, or an isoform thereof; or green-fluorescent protein, or isoform thereof, optionally wherein the exogenous antigen comprises the amino acid sequence set forth in any one of SEQ ID NOS: 14-16, or a sequence having at least 80% identity thereto. The cell membrane signaling peptide of the one or more cell of a second mammalian species may comprise at least one transmembrane domain; bind to a signal recognition protein (SRP); target the antigen to the endoplasmic reticulum (ER) membrane; introduce a lipid anchor; and/or localize the antigen to the cell membrane.

The cell membrane signaling peptide of the one or more cell of the second mammalian species may introduce a lipid anchor, optionally wherein the lipid anchor is glycosylphosphatidylinositol (GPI). The cell membrane signaling peptide may comprise an amino acid sequence selected from any one of SEQ ID NOS: 1-4 or 8, or an amino acid sequence having at least 80% identity thereto.

The cell membrane anchor protein of the one or more cell of a second mammalian species may comprise at least one transmembrane domain; localize the antigen to cell surface of the cell of a second mammalian species; and/or is glycosylated or is not glycosylated.

The cell membrane anchor protein of the one or more cell of a second mammalian species may comprise one or more transmembrane domain selected from a CD8 transmembrane domain, ICAM1 transmembrane domain, E-cadherin transmembrane domain, CLDN6 transmembrane domain, ITGB1 transmembrane domain, ITGB2 transmembrane domain, JAM-B transmembrane domain, NCAM-1 transmembrane domain, MUC-4 transmembrane domain, PDGFRA transmembrane domain, or fragment thereof each. The cell membrane anchor protein of the one or more cell of the second mammalian species may be selected from CD8, ICAM1, E-cad, CLDN6, ITGB1, ITGB2, JAM-B, NCAM-1, MUC-4, PDGFRA, or a fragment thereof each. The cell membrane anchor protein of the one or more cell of the second mammalian species may comprise an amino acid sequence comprising the sequence set forth in any one of SEQ ID NO:9-13, or an amino acid sequence having at least 80% identity thereto.

The antigen-binding protein, cell membrane signaling peptide, and/or a cell membrane anchor protein of the cell of the second mammalian species may be connected with linker, optionally wherein the linker comprises the amino acid sequence set forth in any one of SEQ ID NOS: 5-7.

The one or more cell of the first mammalian species may a human cell and the one or more cell of the second mammalian species may be a cell of a non-human mammalian species.

The one or more cell of the first mammalian species may be a cell of a non-human mammalian species and the one or more cell of the second mammalian species may be a human cell.

The one or more cell of the non-human mammalian species may be selected from: a rodent cell, optionally selected from a mouse cell or rat cell; a non-human primate cell, optionally selected from a monkey cell, chimpanzee cell, gorilla cell, orangutan cell, rhesus macaque cell, marmoset cell or bonobo cell); or an ungulate cell, optionally selected from a pig cell, horse cell, cattle cell, sheep cell, goat cell, or donkey cell.

The one or more cell of the first mammalian species or the one or more cell of the second mammalian species may be a stem cell, optionally an embryonic stem cell. The one or more cell of the first mammalian species or the one or more cell of the second mammalian species may be an induced pluripotent stem cell (iPSC), embryonic stem cell (ESC), or epiblast-derived stem cell (EpiSCs). The one or more cell of the first mammalian species may be an iPSC and the one or more cell of the second mammalian species is an ESC, or the one or more cell of the first mammalian species may be an ESC and the one or more cell of the second mammalian species is an iPSC. The one or more cell of the non-human mammalian species may be organogenesis-disabled. The population of cells may cells may be a chimeric embryo or a chimeric blastocyst.

Provided herein is a method of generating an organ, organoid, or tissue mass comprising of a first mammalian species, the method comprising the steps of: providing a population of one or more cell of the first mammalian species comprising an antigen-recognition domain at the cell membrane, and providing a population of one or more cell of a second mammalian species comprising cells expressing an antigen at the cell membrane; or providing a population of one or more cell of the first mammalian species comprising an antigen at the cell membrane, and providing a population of one or more cell of a second mammalian species comprising cells expressing an antigen-binding protein at the cell membrane, whereby an interspecies chimera is generated, whereby an interspecies chimera is generated, and culturing the interspecies chimera to under conditions that allow the interspecies chimera to develop into the organ, organoid, or tissue mass of the cell of a first mammalian species. The first mammalian species may be a human, and the second mammalian species may be a non-human mammalian. The interspecies chimera may be a chimeric blastocyst.

Provided herein is an organ, organoid, or tissue, which is developed from a population of cells as described herein, or by a method as described herein. The organ, organoid, or tissue mass may be a kidney, liver, heart, lung, pancreas, muscle, stomach, intestine, spleen, bladder, reproductive organs, bone marrow, or skin. The organ, organoid, or tissue may be a human organ, human organoid, or human tissue, and optionally may comprise more than 90% human cells.

Other technical features may be readily apparent to one skilled in the art from the following figures, descriptions, and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects of the present disclosure are illustrated by way of example in which like reference numerals indicate similar elements and in which:

FIGS. 1A-1U shows that cell adhesion is a component of the xenogeneic barrier and can be overcome in vitro using a synthetic nanobody adhesion system. FIG. 1A shows in vitro cultured mouse embryos at Day 1 and Day 3 after injection of hPSCs (red fluorescent signal). FIG. 1B shows immunofluorescence staining for tight junction (ZO1) and adherens junction (E-Cadherin) markers in 2D co-cultured mouse and human PSCs. Human and mouse PSCs were distinguished from one another by red fluorescent labeling of hPSCs (red fluorescent channel not shown) and the larger size of hPSCs as compared to mPSCs. FIG. 1C shows representative electron micrographs of co-cultured mouse and human PSCs. Mouse and human PSCs were distinguished via correlative light-electron microscopy (CLEM), which involves taking fluorescent images of fluorescently labeled hPSCs and mPSCs co-cultured in gridded plates, followed by correlation of colony location and morphology between these images and subsequent electron micrographs. FIG. 1D shows a schematic of the doublet flow cytometry assay. This assay involves combining 200,000 cells each from two PSC lines expressing different fluorescent markers (which may be from the same or different species) then co-culturing these cell lines in suspension on an orbital shaker set to 80 rpm for one hour. The cell suspension is then analyzed via flow cytometry, with a gating strategy that excludes both single cells and larger aggregates to focus only on doublets. Cell doublets with both red and green fluorescence are assumed to be composed of one cell from each line. FIG. 1E depicts results of an interspecies doublet assay using flow cytometry, showing a significant reduction in cell adhesion (as measured by the percentage of dual-colored doublets) between rodent and human cells as compared to adhesion between cells of the same or closely related species (n=6 independent biological replicates). FIG. 1F depicts results of a mouse-human doublet assay, which was performed between mouse and human cells cultured in the naïve state (4CL medium), in the primed state (NBFR medium), and after 7 days of random differentiation (N2B27+10% FBS) (n=5 biological replicates). FIG. 1G shows a schematic of the 3D co-aggregation assay, which involves combining 7500 cells each from two fluorescently labeled PSC lines in an Aggrewell 800 plate. This plating density generates aggregates of 25+25 cells per microwell. Cells are co-cultured in the Aggrewell plate for 24 hours and allowed to form small aggregates, at which point they are imaged in situ to determine the percentage of intermixed and segregated aggregates. FIG. 1H shows representative images of the interspecies 3D co-aggregation assay between mouse-mouse, human-human, and mouse-human cell combinations. FIG. 1I shows quantification of the interspecies co-aggregation assay, measuring the percentage of aggregates containing significant intermixing between cell lines (n=5 biological replicates). FIG. 1J shows a schematic of wash assay protocol. 100,000 red fluorescent cells of each PSC line of interest are plated into Matrigel-coated cell culture inserts in an 8-well ibidi chamber slide (the remainder of the chamber slide is uncoated) and allowed to generate a confluent monolayer. The following day, the inserts are removed and 100,000 membrane-GFP-expressing mPSCs are added to each well of the culture chamber. After 30 minutes, the chambers are washed with PBS to remove non-adherent cells, then fixed in 4% PFA and imaged on a confocal microscope. FIG. 1K shows representative images of the wash assay showing red fluorescent mEpiSC and hPSC monolayers with attached GFP-GPI mEpiSCs after washing.

FIG. 1L shows quantification of interspecies wash assay results by measuring the average number of attached GFP-GPI mEpiSCs per mm (Chen et al., Proc Natl Acad Sci (1993) 90, 4528-4532) 2 (n=8 biological replicates). FIG. 1M depicts the strategy for increasing adhesion between mouse and human PSCs via binding between membrane-localized nanobody and antigen. FIG. 1N shows a summary of results from the mouse-human doublet flow cytometry assay, showing a significantly higher percentage of dual-color doublets between Mouse+Nano-GPI hiPSCs and Mouse+Nano-CD8 hESCs as compared to Mouse+WT hiPSCs (n=6 biological replicates). FIG. 1O shows representative images of the 3D co-aggregation assay between mouse and human PSCs, with and without nanobody adhesion. FIG. 1P shows a summary of 3D co-aggregation assay results for mouse-human aggregation. There were significantly more intermixed aggregates in the Mouse+Nano-GPI hiPSC and Mouse+Nano-CD8 hESC conditions than the Mouse+WT hiPSC condition, n=6 independent biological replicates. FIG. 1Q shows representative images of the wash assay showing WT hiPSC, Nano-GPI hiPSC, and Nano-CD8 hESC monolayers with attached GFP-GPI mEpiSCs. FIG. 1R shows a summary of wash assay results, showing a significant increase in attached GFP-GPI-mEpiSCs on Nano-GPI hiPSC and Nano-CD8 hESC monolayers as compared to WT hiPSC monolayers (n=5 biological replicates). FIG. 1S shows representative images of the lumen formation assay, which measures the ability of human PSCs to undergo polarization and contribute to the formation of a lumen in mixed mouse-human aggregates. Aggregates were formed in a 10% Matrigel overlay and stained with phalloidin to mark actin accumulation on the apical surface of cells. FIG. 1T shows quantification of the average number of human cells in each chimeric aggregate during the lumen formation assay. FIG. 1U shows average percentage of polarized human cells in chimeric aggregates. Polarized cells were identified by apical actin accumulation and direct contact with the central lumen.

FIGS. 2A-2H show that nanobody adhesion significantly increases human PSC contribution to interspecies chimeric embryos at mid-gestation. FIG. 2A shows representative fluorescent images of an E12.5 intraspecies chimeric embryo generated by injection of Nano-GPI mESCs into GFP-GPI mouse blastocysts. Chimeric embryos appeared grossly normal at mid-gestation. FIG. 2B shows intraspecies chimeric embryos with Nano-GPI mESC donor cells were able to give rise to live-born pups, shown here on postnatal day 8. Donor cells were derived from the C57BL/6 mouse strain, so chimera contribution can be visualized by coat color change. FIG. 2C depicts a schematic of interspecies chimera experiments involving injection of Nano-GPI hiPSCs into GFP-GPI mouse blastocysts, followed by embryo transfer to a surrogate mother and harvesting embryos for analysis at E8.5. Embryos were then analyzed via immunohistochemistry and genomic qPCR for human-specific mitochondrial markers. FIG. 2D show fluorescence stereomicroscopy of a representative interspecies chimeric embryo at E8.5. Donor cells were Nano-GPI hiPSCs labeled with nuclear mKO fluorescence. GFP channel not shown. FIG. 2E shows mitochondrial genomic qPCR quantification of human contribution to representative E8.5 chimeric embryos with primers recognizing a human-specific mitochondrial DNA sequence, as compared to a standard curve of known human/mouse cell mixtures. FIG. 2F shows percentage of Nano-GPI hiPSC interspecies chimeric embryos per embryo transfer above the positive qPCR threshold (equivalent to 1:10,000 human cells) at E8.5, as compared to the percentage of positive control hiPSC chimeric embryos per transfer. FIG. 2G shows comparison of the average percentage of human DNA per embryo between Nano-GPI hiPSC chimeric embryos and control hiPSC chimeric embryos (n=37 control hiPSC embryos and n=43 Nano-GPI hiPSC embryos). Percentage of human DNA was calculated using a linear regression equation derived from the standard curve of human/mouse cell ratios. FIG. 2H shows immunohistochemistry (IHC) staining of frozen sections from E8.5 Nano-GPI hiPSC interspecies chimeric embryos. Tissue sections were co-stained with ectoderm/mesoderm/endoderm markers and an antibody recognizing a human-specific mitochondrial antigen.

FIGS. 3A-3Q show expression of a membrane-localized anti-GFP nanobody to overcome the interspecies adhesion barrier, related to FIGS. 1A-1U. FIG. 3A depicts a proposed mechanism for elimination of human cells in chimeric embryos due to a lack of cell adhesion during epiblast formation. FIG. 3B shows representative images of the interspecies 3D co-aggregation assay between primate species including human, chimpanzee, and rhesus macaque. FIG. 3C shows quantification of the interspecies co-aggregation assay between chimpanzee, rhesus macaque, and human (n=5 biological replicates). FIG. 3D shows representative images of the interspecies 3D co-aggregation assay between pig and human. FIG. 3E shows quantification of the interspecies co-aggregation assay between pig and human (n=6 biological replicates). FIG. 3F depicts the gating strategy for the doublet flow cytometry assay. FIG. 3G shows results of the doublet flow cytometry assay with human, chimpanzee, and rhesus macaque PSCs, showing no significant interspecies differences in adhesion on a single-cell level (n=5 biological replicates). FIG. 3H depicts a schematic of GPI and CD8 membrane-anchoring strategies for localization and orientation of an anti-GFP nanobody. FIG. 2I shows immunostaining with an anti-camelid antibody showing membrane localization of nanobody expression. FIG. 3J shows GFP binding assay demonstrating that membrane-embedded nanobody is capable of binding to free-floating GFP in the media after treatment with purified GFP protein. FIG. 3K shows that nanobody expression does not affect mESC doubling time (n=3 independent biological replicates.) FIG. 3L shows immunostaining of pluripotency factors OCT4 and SOX2 in Nano-GPI and Nano-CD8 mESCs. FIG. 3M shows teratoma formation assay showing that Nano-GPI and Nano-CD8 mESCs can differentiate into cells derived from all three germ layers after injection into immunodeficient mice. FIG. 3N shows representative results of the doublet flow cytometry assay for Mouse+Mouse, Mouse+WT hPSC, Mouse+Nano-GPI hPSC, and Mouse+Nano-CD8 hPSC conditions. FIG. 3O shows representative images of the 3D co-aggregation assay performed between human and pig PSCs with and without Nano-GPI expression in the human cells. FIG. 3P shows quantification of the human-pig 3D co-aggregation assay, showing an increase in highly intermixed aggregates in the Nano-GPI condition (n=6 biological replicates). FIG. 3Q shows results of the doublet flow cytometry assay performed between human-human, pig-pig, and human-pig with and without Nano-GPI expression.

FIGS. 4A-4I depict further details of intraspecies and interspecies chimera formation, related to FIGS. 2A-2H. FIG. 4A shows fluorescence microscopy images of P0 pups from the GFP-GPI mouse strain demonstrating strong GFP expression in all tissues. FIG. 4B shows representative fluorescence microscopy images of additional E12.5 intraspecies chimeric embryos generated with Nano-GPI mESC donor cells. FIG. 4C shows immunohistochemistry (IHC) staining of frozen sections from E12.5 intraspecies chimeric embryos generated with Nano-GPI mESC donor cells. Tissue sections were co-stained with ectoderm/mesoderm/endoderm markers and an anti-mKO antibody to mark donor cell contribution. FIG. 4D shows fluorescent images of additional E8.5 interspecies chimeric embryos generated by injecting Nano-GPI hiPSCs into GFP-GPI mouse blastocysts. GFP channel not shown. FIG. 4E shows mitochondrial genomic qPCR with primers recognizing a second human-specific mitochondrial sequence in representative E8.5 Nano-GPI hiPSC chimeric embryos, as compared to known human/mouse cell ratios. FIG. 4F shows percentage of Nano-GPI hiPSC chimeric embryos per embryo transfer above the positive qPCR threshold (equivalent to 1:10,000 human cells) for human contribution at E8.5 as compared to the percentage of positive control hiPSC chimeric embryos, calculated using Mitochondrial Element 2.

FIG. 4G shows average percentage of human DNA per embryo in control and Nano-GPI hiPSC interspecies chimeric embryos, calculated using Mitochondrial Element 2 qPCR. FIG. 4H shows representative images of E8.5 intraspecies chimeric embryos generated by heterochronic injection of primed Nano-GPI mEpiSCs into blastocyst-stage GFP-GPI embryos. GFP channel not shown.

FIG. 41 shows immunohistochemistry (IHC) staining of frozen sections from E8.5 intraspecies chimeric embryos generated with stage-mismatched Nano-GPI mEpiSC donor cells. Tissue sections were co-stained with ectoderm/mesoderm/endoderm markers and an anti-mKO antibody to mark donor cell contribution.

FIGS. 5A-5B show that cytoskeleton-bound cell adhesion proteins (e.g., ICAM1) efficiently facilitate binding and cell adhesion. FIG. 5A shows a schematic diagram of wash assay adapted to verify ICAM1 as a cell membrane anchor protein. FIG. 5B shows GFP signal after the wash assay, with or without GFP nanobody expression in mESCs.

### DETAILED DESCRIPTION

The following detailed description references the accompanying drawings that illustrate various aspects of the present disclosure. The drawings and description are intended to describe aspects and aspects of the present disclosure in sufficient detail to enable those skilled in the art to practice the present disclosure. Other components can be utilized and changes can be made without departing from the scope of the present disclosure. The following description is, therefore, not to be taken in a limiting sense.

The generation of transplantable organs to address the global shortage of human donors is a key objective in regenerative medicine. Interspecies blastocyst complementation is a promising technique, but its effectiveness is hampered by limited human cell integration into animal chimeras due to various xenogeneic barriers. The current disclosure is based on the unexpected discovery that differential cell-cell adhesion, crucial for tissue separation and boundary formation during development, is a major barrier to interspecies chimerism. Using a series of interspecies pluripotent stem cell (PSC) cell adhesion assays, it is demonstrated here that this barrier is more pronounced between evolutionarily distant species and during primed pluripotency. To overcome this barrier, the current disclosure takes a synthetic biology approach, using membrane-localized antigen and antigen-binding protein interactions to enhance adhesion between mouse and human PSCs in vitro. Importantly, these studies demonstrate that a synthetic cell adhesion strategy significantly increases the chimeric contribution of human cells in mouse embryos in vivo. This approach has the potential to broaden the scope of interspecies organogenesis.

In an aspect, the current disclosure encompasses the design of a range of interspecies co-culture assays to investigate cell adhesion among PSCs. These include single-cell level analysis using doublet flow cytometry, and more complex structures like aggregates and lumens that mimic natural cell assemblies. Utilizing these assays, it is shown herein that cell adhesion barriers are present between evolutionarily distant species. Without being bound to theory, these barriers are notably more evident during primed pluripotency compared to naïve pluripotency and early differentiation.

In an aspect, the current disclosure encompasses synthetic biology techniques to artificially enhance cell adhesion. This innovative cell adhesion system employs engineered cells to display antigen-binding proteins (e.g., nanobodies) and their corresponding antigens on the outer plasma membrane. This modification markedly improved the interaction between human and mouse PSCs in vitro, and significantly boosted the integration of human PSCs into mouse embryos, enhancing chimerism. This validates the effectiveness of the disclosed methods in promoting adhesion among cells that typically do not adhere well to one another. This study also takes this approach a step further by applying it to enhance adhesion between pluripotent cells of different species (e.g., human and non-human). This enhancement significantly improves the cell adhesion between PSCs from different species and the chimeric contribution of hPSCs to mouse embryos. The enhanced cell adhesion further improves survival of in interspecies chimeric embryos, for example human cells in mouse-human chimeric embryos.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to #1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., +10%, +9%, +8%, +7%, +6%, +5%, +4%, +3%, +2%, or +1%.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence, one may replace it by: a polypeptide represented by an amino acid sequence comprising a sequence that has at least about 80% sequence identity or similarity with the recited amino acid sequence. Another preferred level of sequence identity or similarity is about 85%. Another preferred level of sequence identity or similarity is about 90%. Another preferred level of sequence identity or similarity is about 95%. Another preferred level of sequence identity or similarity is about 98%. Another preferred level of sequence identity or similarity is about 99%.

Each amino acid sequence described herein by virtue of its identity or similarity percentage with a given amino acid sequence respectively has in a further preferred aspect an identity or a similarity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [b162], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and Val to Ile or Leu.

An “individual” or “subject,” as used interchangeably herein, is a mammal. In certain aspects, the individual or subject is a human.

### II. Engineering of cell for enhanced cellular adhesion

In an aspect, provided herein is a population of cells, comprising one or more cell of a first mammalian species and one or more cell of a second mammalian species, wherein the one or more cell of the first mammalian species comprises an exogenous nucleic acid encoding an antigen-binding protein that specifically binds to an antigen on the cell of a second mammalian species. The antigen may be exogenous or endogenous to the cell of a second mammalian species.

In an aspect, provided herein is a population of cells, comprising one or more cell of a first mammalian species and one or more cell of a second mammalian species, wherein the one or more cell of a second mammalian species comprises an exogenous nucleic acid encoding an antigen-binding protein that specifically binds to an antigen on a cell of a first mammalian species. The antigen may be exogenous or endogenous to the cell of a first mammalian species.

### (a) Cell Types

A cell as described herein (e.g., as in a population of cells comprising one or more cells, or a method comprising a cell or population of cells, as described herein) may be a cell of a first mammalian species or a cell of a second mammalian species. The first mammalian species and second mammalian species will be different species.

A cell of a first mammalian species, as described herein, may be a stem cell. In some embodiments, the cell of the first mammalian species may be selected from a totipotent stem cell, pluripotent stem cell, multipotent stem cell, oligopotent stem cell, or unipotent stem cell. The totipotent stem cell of the first mammalian species may be a blastomere or 4-cell embryo. The pluripotent stem cell of the first mammalian species may be an embryonic stem cell or an induced stem cell. The pluripotent stem cell of the first mammalian species may be naïve or primed. The pluripotent stem cell of the first mammalian species may bean epiblast-derived stem cell. A multipotent stem cell of a first mammalian species may be an endothelial stem cell. The unipotent cell of a first mammalian species as described herein may be a progenitor cell. The human progenitor cell may be selected from a kidney progenitor cell, hepatic progenitor cell, cardiac progenitor cell, intestinal progenitor cell, lung progenitor cell, epidermal progenitor cell, muscle progenitor cell, mesenchymal progenitor cell, olfactory progenitor cell, hematopoietic progenitor cell, neural progenitor cell, or any progenitor cell of the desired organ, organoid, or tissue.

A cell of a second mammalian species, as described herein, may be a stem cell. In some embodiments, the cell of the second mammalian species may be selected from a totipotent stem cell, pluripotent stem cell, multipotent stem cell, oligopotent stem cell, or unipotent stem cell. The totipotent stem cell of the second mammalian species may be a blastomere or 4-cell embryo. The pluripotent stem cell of the second mammalian species may be an embryonic stem cell or an induced stem cell. The pluripotent stem cell of the second mammalian species may be naïve or primed. The pluripotent stem cell of the second mammalian species may bean epiblast-derived stem cell. A multipotent stem cell of a second mammalian species may be an endothelial stem cell. The unipotent cell of a second mammalian species as described herein may be a progenitor cell. The human progenitor cell may be selected from a kidney progenitor cell, hepatic progenitor cell, cardiac progenitor cell, intestinal progenitor cell, lung progenitor cell, epidermal progenitor cell, muscle progenitor cell, mesenchymal progenitor cell, olfactory progenitor cell, hematopoietic progenitor cell, neural progenitor cell, or any progenitor cell of the desired organ, organoid, or tissue.

The one or more cell of a second mammalian species may be organogenesis-disabled, for example is genetically modified to have a dysorganogenetic phenotype or trait. An organogenesis-disabled cell may comprise a disabling of one or more host genes critical for a particular organ's development. The organogenesis-disabled cell of a second mammalian species is preferably completely organogenesis-disabled, but may be partially organogenesis-disabled. An organogenesis-disabled cell may develop an empty organ niche (e.g., developmental niche) during organogenesis. For example, the cell of a second mammalian species may be anephrogenic, ahepatogenic, apancreatic (e.g., lack the ability to develop a kidney, liver, or pancreas), or lack the ability to develop another organ. A population of cells comprising one or more organogenesis-disabled cell of a second mammalian species may have its dysorganogenetic phenotype corrected by complementation with a cell of a second mammalian species comprising an organogenetic phenotype for the disabled organ. In other words, the cell of a second mammalian species may fill the developmental niche for a particular organ that has been disabled in the cell of a second mammalian species, or the cell of a second mammalian species may be organogenesis-enabled.

In an aspect the population of cells is a chimeric embryo. The chimeric embryo may be at any stage of embryonic development, wherein the stage is selected from a blastocyst stage, implantation stage, gastrulation stage, neurulation stage, organogenesis stage, or fetal development stage. In some embodiments, the population of cells is a chimeric blastocyst.

A chimeric embryo may be an interspecies chimeric embryo. For example, a chimeric embryo may be a developing embryo comprising one or more cells of a second mammalian species and one or more cells of another mammalian species. The chimeric embryo may comprise one or more cells from at least a second mammalian species and a second mammalian species, optionally a third mammalian species, and further optionally a fourth mammalian species. As a chimeric embryo develops, the one or more cell of a second mammalian species may fill the vacated developmental organ niche, or may enrich in a target organ.

A cell of a first or second mammalian species as described herein may be selected from a human cell or non-human mammalian cell. The non-human mammalian cell may be a rodent cell, optionally selected from a mouse cell or rat cell; a non-human primate cell, optionally selected from a monkey cell, chimpanzee cell, gorilla cell, orangutan cell, rhesus macaque cell, marmoset cell or bonobo cell); an ungulate cell, optionally selected from a pig cell, horse cell, cattle cell, sheep cell, goat cell, or donkey cell; or another mammal. In some embodiments, a cell of the first species is a human cell and the cell of the second species is a non-human mammalian cell. In some embodiments, a cell the second species is a human cell and the cell of the first species is a non-human mammalian cell.

In an aspect, the chimeric embryo may comprise at least 2-cells, at least 4-cells, at least 8-cells, at least 16-cells, at least 32-cells, at least 64-cells, at least 128 cells, or more. The respective percentage of cells of the first and second mammalian species in the population of cells may vary depending on, for example, the developmental stage of the embryo, the duration of chimerism, and the cellular growth rate of the cells of the first and second mammalian species in the embryo.

The population of cells may comprise one or more cell of a first mammalian species comprising about 1% to about 5%, about 5% to about 10%, about 10% to about 15%, about 15% to about 20%, about 20% to about 30%, about 30% to about 40%, about 40% to about 50%, about 50% to about 60%, about 60% to about 70%, about 70% to about 80%, about 80% to about 85%, about 90% to about 95%, or greater than 95% of the cells of the chimeric embryo. In some embodiments, the population of cells may comprise one or more cell of a first mammalian species comprising about 1% or more, about 5% or more, about 10% or more, about 15% or more, about 15% or more, about 20% or more, about 30% or more, about 40% or more, about 50% or more, about 60% or more, about 70% or more, about 80% or more, about 85% or more, about 90% or more, about 91% or more, about 92% or more, about 93% or more, about 94% or more, about 95% or more, about 96% or more, about 97% or more, about 98% or more, or about 99% or more of the cells of the chimeric embryo.

The population of cells may comprise one or more cell of a second mammalian species comprising about 1% to about 5%, about 5% to about 10%, about 10% to about 15%, about 15% to about 20%, about 20% to about 30%, about 30% to about 40%, about 40% to about 50%, about 50% to about 60%, about 60% to about 70%, about 70% to about 80%, about 80% to about 85%, about 90% to about 95%, or greater than 95% of the cells of the chimeric embryo. In some embodiments, the population of cells may comprise one or more cell of a second mammalian species comprising about 1% or more, about 5% or more, about 10% or more, about 15% or more, about 15% or more, about 20% or more, about 30% or more, about 40% or more, about 50% or more, about 60% or more, about 70% or more, about 80% or more, about 85% or more, about 90% or more, about 91% or more, about 92% or more, about 93% or more, about 94% or more, about 95% or more, about 96% or more, about 97% or more, about 98% or more, or about 99% or more of the cells of the chimeric embryo.

### (b) Nucleic Acids

In an aspect, a cell of a first mammalian species or a cell of a second mammalian species, as described herein (e.g., as in a population of cells, or a method comprising a cell or population of cells), comprises a nucleic acid encoding one or more protein selected from an antigen-binding protein, antigen, cell membrane signal peptide, or cell membrane anchor protein. The nucleic acid encoding the protein may be an exogenous nucleic acid or recombinant nucleic acid.

The nucleic acid encoding the protein may comprise a nucleic acid sequence encoding an antigen-binding protein and one or more cell membrane signal peptide and/or one or more cell membrane anchor protein. The nucleic acid encoding the protein may comprise a nucleic acid sequence encoding an antigen and one or more cell membrane signal peptide and/or one or more cell membrane anchor protein. Multiple cell membrane anchor proteins, such as a two, three, or four, may be encoded along with an antigen or antigen-binding protein. The antigen-binding protein may be on the cell of a first mammalian species and the antigen on the cell of a second mammalian species, or the antigen-binding protein may be on the cell of a second mammalian species and the antigen on the cell of a first mammalian species.

In a further aspect, the nucleic acid sequence encoding the protein comprises a nucleic acid sequence encoding a linker. A linker may attach the expressed protein (e.g., an antigen-binding protein or antigen) to the cell membrane signal peptide, or to the cell membrane anchor protein, or both. Multiple linkers can be used, which can be the same or the linkers may be different from each other. A linker may also attach the cell membrane signal peptide to the cell membrane anchor protein. In some embodiments, the linker comprises an amino acid sequence that is rich in glycine, serine, alanine, or a combination thereof. In some embodiments, the peptide linker may be of from 1 to about 30 amino acid residues in length, inclusive. In some embodiments, the peptide linker may be 2-3 amino acid residues in length, 4-5 amino acid residues in length, 6-7 amino acid residues in length, 8-9 amino acid residues in length, 10-11 amino acid residues in length, 12-13 amino acid residues in length, 14-15 amino acid residues in length, 16-17 amino acid residues in length, 18-19 amino acid residues in length, or greater than 20 amino acid residues in length. In some embodiments, the linker comprises the amino acid sequence of GGGG/S. In some embodiments, the linker comprises an amino acid selected from any one of SEQ ID NOS: 5-7, or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with the sequence set forth in any one of SEQ ID NOS: 5-7.

In an aspect, the nucleic acid sequence encoding the protein may further comprise one or more regulatory sequences. The nucleic acid encoding the protein may be operably linked to one or more transcription regulatory sequences. “Transcriptional regulatory elements” refer to any nucleotide sequence that influences transcription initiation and rate, or stability and/or mobility of a transcript product. Regulatory sequences include, but are not limited to, promoters, promoter control elements, enhancers, protein binding sequences, 5′ and 3′ UTRs, transcriptional start sites, termination sequences, polyadenylation sequences, introns, etc. Such transcriptional regulatory sequences can be located either 5′-, 3′-, or within the coding region of the nucleic acid encoding the protein and can be either promote (positive regulatory element) or repress (negative regulatory element) transcription. The nucleic acid may comprise one or more transposase ITR. The one or more transoposase ITR may be a piggybac transposase ITR. A transposase system may be used to enhance genetic modification or integration.

The nucleic acid as described herein may comprise one or more regulatory sequence, transcription regulatory sequence, or other nucleic acid component of a vector as described herein, such as disclosed in any one of SEQ ID NOS: 46-113. The nucleic acid sequence may comprise a regulatory sequence having the nucleic acid sequence of a regulatory sequence within any one of SEQ ID NOS: 46-113, or a variant thereof having a percent identity to any one of SEQ ID NOS: 46-113 that conserves function of the regulatory sequence.

In an aspect, the transcription regulatory sequence is a promoter, an enhancer, or both. A regulatory sequence can be a promoter. A disclosed promoter can comprise a ubiquitous promoter, a constitutive promoter, or a tissue specific promoter. In an aspect, a disclosed promoter can be operably linked to a nucleic acid sequence encoding a protein as described herein. In an aspect, a disclosed promoter can be a promoter/enhancer. An enhancer element is a nucleic acid sequence that functions to enhance transcription. In an aspect, a disclosed promoter can be an endogenous promoter. In an aspect, a disclosed endogenous promoter can be an endogenous promoter/enhancer. In an aspect, a disclosed promoter or a disclosed promoter/enhancer can be used for constitutive and efficient expression of the disclosed nucleic acid. In an aspect, a disclosed promoter or a disclosed promoter/enhancer can be used for inducible and efficient expression of a disclosed antigen or antigen-binding protein. A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter, CAG promoter). In some cases, the promoter can be a spatially restricted and/or temporally restricted promoter (e.g., a tissue specific promoter, a cell type specific promoter, etc.). Promoters and enhancer sequences are known to the art.

The promoter can be chosen so that it will function in the target cell(s) of interest. Cell- or tissue-specific promoters refer to promoters that have activity in only certain cell types. The use of a cell- or tissue-specific promoter in a nucleic acid expression cassette can restrict unwanted transgene expression in the unaffected tissues or cells as well as facilitate persistent transgene expression by escaping from transgene induced host immune responses. Tissue specific promoters include, but are not limited to, kidney-specific promoters, liver-specific promoters, heart-specific promoters, lung-specific promoters, pancreas-specific promoters, muscle-specific promoters (e.g., skeletal muscle-specific promoters), or specific to a cell, organ, organoid, or type type described herein.

In other aspects, the promoter can be a constitutive promoter. Constitutive promoters refer to promoters that allow for continual transcription of its associated gene. Constitutive promoters are always active and can be used to express genes in a wide range of cells and tissues. Examples of constitutive promoters include, but are not limited to, U6 promoter, a CMV major immediate-early enhancer/chicken beta-actin promoter, a cytomegalovirus (CMV) major immediate-early promoter, an Elongation Factor 1-a (EF1-a) promoter, a simian vacuolating virus 40 (SV40) promoter, an AmpR promoter, a PgK promoter, a human ubiquitin C gene (Ubc) promoter, a MFG promoter, a human beta actin promoter, a CAG promoter, a EGRI promoter, a FerH promoter, a FerL promoter, a GRP78 promoter, a GRP94 promoter, a HSP70 promoter, a b-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous promoters. In an aspect, the constitutively active promoter can comprise human b-actin, human elongation factor-la, chicken b-actin combined with cytomegalovirus early enhancer, cytomegalovirus (CMV), simian virus 40, or herpes simplex virus thymidine kinase.

Inducible promoters refer to promoters that can be regulated by positive or negative control. Factors that can regulate an inducible promoter include, but are not limited to, chemical agents (e.g., the metallothionein promoter or a hormone inducible promoter), temperature, and light. As discussed above, a disclosed promoter can be an endogenous promoter. Endogenous refers to a disclosed promoter or disclosed promoter/enhancer that is naturally linked with its gene. In an aspect, a disclosed endogenous promoter can generally be obtained from a non-coding region upstream of a transcription initiation site of a gene.

Methods of introducing the disclosed nucleic acids into the cell of the first or second species are well known in the art, and may include viral transfection, plasmid transduction, CRISPR related technologies etc. In an aspect, the cell of the first or second species or both may further comprise one or more additional genetic modifications. In an aspect, the one or more genetic modification may promote the growth of cell and cell division of the cells of one species, over the growth and cell division of cells of the other species. In an aspect, the one of more genetic modification may comprise a deletion in a developmental gene. In an aspect, the one of more genetic modification may comprise induction of organo-genesis deficiency.

The nucleic acid as described here may be a vector as described herein, either fully or partially. The nucleic acid may comprise the nucleotide sequence set forth in any one of SEQ ID NOS: 46-113, a fragment thereof, or a functionally equivalent variant sequence thereof.

(c) Antigen-binding protein

The antigen present at the surface of the cell to be adhered to specifically binds to the antigen-binding protein of the other species. Cells engineered to express antigens in the outer plasma membrane can significantly improve the adhesion between cells of different species, when the cell from the other species expresses an antigen-binding protein at its surface to mediate the interaction. The antigen-binding domain may bind to an endogenous antigen on the cell of the other mammalian species, or an exogenous antigen on the cell of the other mammalian species.

An antigen-binding protein may be selected from an antibody or antigen-binding fragment thereof. The antibody may be a conventional antibody (e.g., an IgG antibody comprising a Fab and Fc) derived from a rodent (e.g., mouse or rodent), human, non-human primate, or ungulate. The may be a camelid-derived antibody or a shark-derived antibody. The antibody may polyclonal or monoclonal. The antibody may be humanized or not humanized. The antibody may be from the first mammalian species, or second mammalian species. The antigen-binding protein may be a fragment of antibody comprising the antigen-binding domain.

The antigen-binding protein may be a single domain antibody. The antigen-binding protein may comprise the VHH domains or VLL domains of an antibody. The antigen-binding protein may be a nanobody. Exemplary nanobodies are provided herein, including the anti-GFP nanobodies of the Examples.

An antigen-binding protein may be selected from a fragment variable domain (FV) (e.g., comprising a VH and VL); a fragment antigen binding (Fab) domain (e.g., comprising a VH-CHI and VL-CL1); a Fab′ (e.g., a Fab with part of the hinge), a (Fab′) 2 (e.g., two Fab regions with a hinge), or a half-IgG (e.g., an rIgG, or having only the left or right half of an antibody such as VH-CH1-CH2-CH3 and VL-CL). The antigen-binding protein may comprise antibody fragments linked together with linkers. The antigen-binding protein may be selected from: a single-chain fragment variable domain (scFv) (e.g., VH and VL connected with a linker); a diabody or dscFv (e.g., a divalent scFV comprising two scFv fragments connected with a linker); a triabody or tri-scFv (e.g., three scFv fragments connected with a linker); an single chain Fab (scFab) (e.g., a Fab element connected by a linker instead of hinge); a minibody (e.g., two scFv fragments connected with with CH3 regions, optionally with a hinge or linker); or an scFv-Fc (e.g., two scFv fragments linked to partial Fc region).

In some embodiments, the antigen-binding protein binds to an endogenous antigen of the cell type of the first mammalian species or second mammalian species (e.g., human or non-human cell type). In some embodiments, the antigen-binding protein binds to an exogenous antigen of the cell type of the first mammalian species or second mammalian species (e.g., human or non-human cell type). In some embodiments, the antigen-binding protein binds to green fluorescent protein, or an isoform thereof.

In some embodiments, an antigen-binding protein comprises the amino acid sequence set forth in any one of SEQ ID NOS: 17-45, or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with the sequence set forth in any one of SEQ ID NOS: 17-45.

In some embodiments, an antigen-binding protein may have the amino acid sequence of a component of a vector that binds to the antigen as described herein, such as disclosed in any one of SEQ ID NOS: 46-113, or a variant thereof having a percent identity to any one of SEQ ID NOS: 46-113 that conserves function of the antigen-binding protein.

### (d) Antigens

The antigen specifically bound to by the antigen-binding protein may be a protein or a fragment thereof, which is present at the surface of the cell to be adhered to. Cells engineered to express antigens in the outer plasma membrane can significantly improve the adhesion between cells of different species, when the cell from the other species expresses an antigen-binding protein at its surface to mediate the interaction. The antigen be an outer plasma membrane antigen, or surface antigen. In some embodiments, the antigen is present on the cell of a first mammalian species and the antigen-binding protein is present on the cell of a second mammalian species. In some embodiments, the antigen is present on the cell of a second mammalian species, and the antigen-binding protein is present on the cell of a first mammalian species. The cell of the first species may be a human cell, and the cell of the second species may be a non-human mammalian cell.

In an aspect, the antigen may be endogenous to the cell type selected for the first or second mammalian species (e.g., human or non-human cell). For example, an antigen-binding protein may be engineered into a cell of a first mammalian species, wherein the antigen-binding protein specifically binds to an antigen endogenously expressed by a certain cell type of the second mammalian species. The antigen-binding protein may be engineered into a cell of a second mammalian species, wherein the antigen-binding protein specifically binds to an antigen endogenously expressed by a certain cell type of a first mammalian species. The antigen may be endogenously expressed by the first or second mammalian species (e.g., human or non-human mammalian species) totipotent stem cell, pluripotent stem cell, multipotent stem cell, oligopotent stem cell, or unipotent stem cell, as described herein. The antigen may be endogenously expressed by a blastomere, 4-cell embryo, embryonic stem cell, epiblast-derived stem cell, induced pluripotent stem cell, naïve pluripotent stem cell, endothelial stem cell, or progenitor cell of the first or second mammalian species, as described herein. The antigen may be from an embryonic or induced cellular state, and/or a primed or naive cellular state.

The endogenous antigen may be an antigen specific for an embryonic stem cell, optionally wherein the antigen is selected from CD9, CD15/SSEA-1, CD24, CD29 (B1 integrin), CD31 (PECAM-1), CD49b/ITGA2, CD49c/ITGA3, CD49e/ITGA5, CD49f/ITGA6 (integrin A6), CD59, CD90 (Thy-1), CD117 (c-Kit), CD133, CD324 (E-Cadherin), CD326/EpCAM, CD338/ABCG2, Cripto, or Frizzled5. The endogenous antigen may be an antigen specific for a pluripotent stem cell, optionally wherein the antigen is selected from cSSEA1, SSEA3, SSEA4, SSEA5, TRA-1-60, TRA-1-81, or TRA-2-54. Amino acid sequences for endogenous antigens may be found for a specific species or cell type from publicly available databases, e.g., NCBI or UniProt, which provides sequence annotation of domains that are targetable for antigen-binding proteins.

The antigen may be exogenous to the cell type selected for the first or second mammalian species. When the first or second mammalian species is a human, the exogenous antigen may not be an endogenous antigen of that particular human cell type. When the first or second mammalian species is a non-human mammal, the exogenous antigen may not be an endogenous antigen of that particular non-human mammalian cell type. the antigen may be engineered into a cell of a first mammalian species, when the antigen-binding protein is engineered into the cell of a second mammalian species. Or, the antigen may be engineered into a cell of a second mammalian species, when the antigen-binding protein is engineered into the cell of a first mammalian species. In some embodiments, the antigen is exogenous to both the first and second mammalian species, or is exogenous to the specific cell type of each of the first and second mammalian species.

Any protein or polypeptide that can be recognized by an antigen-binding protein, as described herein, can be an antigen engineered into the cell membrane. In some embodiments, the antigen is fluorophore or an isoform thereof. The antigen may be selected from mCherry, or an isoform thereof; or green-fluorescent protein, or isoform thereof. Exemplary antigens are provided herein, including the GFP constructs of the Examples.

The antigen may be an exogenous peptide or polypeptide, which can be natural or synthetic. For example, an antigen-binding protein may be generated against an antigen using techniques known in the art (e.g., immunization of an animal, and recombinant production of the antigen-binding fragment of the antibodies from the immunized animal).

In some embodiments, an antigen comprises the amino acid sequence set forth in any one of SEQ ID NOS: 14-16, or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with the sequence set forth in any one of SEQ ID NOS: 14-16.

In some embodiments, an antigen may have the amino acid sequence of a component of a vector that binds to the antigen-binding protein as described herein, such as disclosed in any one of SEQ ID NOS: 46-113, or a thereof having a percent identity to any one of SEQ ID NOS: 46-113 that conserves function of the antigen.

### (e) Cell Membrane Signaling Peptides

A cell of a first or second mammalian species (e.g., a human cell or non-human cell, as described herein), may comprise a nucleic acid encoding a cell membrane signaling peptide. The cell membrane signaling peptide directs the encoded protein to which it is attached to the cell membrane. The cell membrane signaling protein may bind to a signal recognition protein for directing to the cell membrane. The cell membrane signaling peptide may direct the encoded protein to the membrane of the endoplasmic reticulum (ER). The cell membrane signaling peptide may translocate the encoded protein to the ER. After incorporation into the ER, the encoded protein may be directed to the cell membrane. The cell membrane signaling protein may comprise at least one transmembrane domain. The cell membrane signaling peptide may be an amino acid sequence that directs the expressed protein (e.g., an antigen-binding protein or an antigen) to the cell surface (e.g., the outer cell surface) of the cell of the first or second mammalian species (e.g., human cell or non-human cell). Cell membrane signaling peptides are well known in the art. For example, they are immediately derivable from proteins that are transported to the cell membrane after translation, as can be identified using publicly available databases such as NCBI or UniProt.

When a particular cell membrane anchor protein is utilized, the canonical cell membrane signaling protein for that anchor protein may be used. Alternatively, the canonical cell membrane signaling protein for that anchor protein may not be used. In some embodiments, a cell membrane signaling peptide is selected from an acrosin membrane signaling peptide, a CD8 membrane signaling peptide, an integrin membrane signaling peptide, or a Cldn6 membrane signaling peptide. In some embodiments, the cell membrane signaling peptide is encoded by a component of a vector as described herein, such as disclosed in any one of SEQ ID NOS: 46-113, or a thereof having a percent identity to any one of SEQ ID NOS: 46-113 that conserves function of the cell membrane signaling peptide.

In some embodiments, the cell membrane signaling protein comprises the amino acid sequence set forth in any one of SEQ ID NOS: 1-4, or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with the sequence set forth in any one of SEQ ID NOS: 1-4.

The cell membrane signaling peptide may introduce a lipid anchor into the encoded protein. The lipid anchor may be introduced into the encoded protein as a post-translation modification. The lipid anchor may be a molecule that inserts into the cell membrane, whereby the encoded protein is anchored to the cell membrane. The lipid anchor may comprise one or more glycerol moiety, phosphate moiety, fatty acid moiety, or carbohydrate moiety. The lipid anchor may comprise one or more acyl chains (e.g., sn-1 acyl chain, or sn-2 acyl chain). The lipid anchor may comprise one or more hydrophobic moieties (e.g., a hydrophobic phosphatidyl-inositol). The lipid anchor may comprise one, two, three, or more fatty acid chains. Some or all of the fatty acid chains may be inserted into the cell membrane. The lipid anchor may be a phospholipid. The lipid anchor may be attached to encoded protein at the C-terminus, N-terminus, or to an amino acid or modified amino acid within the encoded protein. The attachment of the lipid anchor to the encoded protein may comprise a linker (e.g., a carbohydrate-containing linker, such as comprising glucosamine or mannose). The attachment may comprise an ethanolamine phosphate bridge.

In some embodiments, the cell membrane signaling peptide introduces a glycosylphosphatidylinositol (GPI) into the encoded protein (e.g., an antigen-binding protein or an antigen). In some embodiments, the cell membrane signaling peptide marks protein for post-translational GPI addition. In some embodiments, the cell membrane signaling peptide is a Thy-1 signaling peptide. In some embodiments, the cell membrane signaling protein comprises the amino acid sequence set forth in SEQ ID NO:8, or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with the sequence set forth in any one of SEQ ID NO:8.

### (f) Cell Membrane Anchor Proteins

A cell membrane anchor protein as described herein may be present on a cell of a first mammalian species or cell of a second mammalian species, as described herein. Such cell membrane anchor proteins localize the antigen-binding protein to cell surface. The cell membrane anchor protein may comprise at least one transmembrane domain (e.g., one, two, three, or more transmembrane domains). The transmembrane domain may comprise a transmembrane a-helix, or transmembrane β-sheet. The cell membrane anchor protein may be biotopic (e.g., single pass) or polytopic (e.g., multipass). The cell membrane anchor protein may be glycosylated, or may be not glycosylated.

Certain cell membrane anchor proteins may be only peripherally, rather than integrally, embedded into the membrane. In a further aspect, the membrane anchor protein associates with or attaches to the cytoskeleton. In some embodiments, the cell membrane anchor protein integrates into the membrane by connecting with the cytoskeleton.

The cell anchor protein may be selected from cluster of differentiation 8 (CD8), intercellular adhesion molecule 1 (ICAM1), cadherin-1 or epithelial cadherin (E-Cad), claudin (CLDN6), or integrin subunit beta 1 (ITGB1). The cell anchor protein may comprise one or more transmembrane domains selected from the transmembrane domains of CD8, ICAM1, E-Cad, CLDN6, or ITGB1.

The anchor protein may be selected from integrin subunit beta 2 (ITGB2), junctional adhesion molecule B (JAM-B), neural cell adhesion molecule 1 (NCAM-1), mucin 4 (MUC-4), or platelet derived growth factor receptor alpha (PDGFRA). The cell anchor protein may comprise one or more transmembrane domains selected from the transmembrane domains of ITGB2, JAM-B, NCAM-1, MUC-4, or PDGFRA.

A CD8 cell anchor protein as described herein (e.g., as encoded by a nucleic acid or as present on a cell of a first mammalian species or cell of a second mammalian species), may be a complete CD8 protein or a fragment thereof comprising an amino acid sequence that is a cell membrane anchor. In some embodiments, the CD8 protein comprises a transmembrane domain from a human CD8 protein. In some embodiments, the CD8 protein comprises a transmembrane domain from a rodent CD8 protein, optionally selected from a mouse CD8 protein or rat CD8 protein. In some embodiments, the CD8 protein comprises a transmembrane domain from a non-human primate CD8 protein, optionally selected from a monkey CD8 protein, chimpanzee CD8 protein, gorilla CD8 protein, orangutan CD8 protein, rhesus macaque CD8 protein, marmoset CD8 protein, or bonobo CD8 protein. In some embodiments, the CD8 protein comprises a transmembrane domain from an ungulate CD8 protein, optionally selected from a pig CD8 protein, horse CD8 protein, cattle CD8 protein, sheep CD8 protein, goat CD8 protein, or donkey CD8 protein.

A ICAM1 cell anchor protein as described herein (e.g., as encoded by a nucleic acid or as present on a cell of a first mammalian species or cell of a second mammalian species), may be a complete ICAM1 protein or a fragment thereof comprising an amino acid sequence that is a cell membrane anchor. In some embodiments, the ICAM1 protein comprises a transmembrane domain from a human ICAM1 protein. In some embodiments, the ICAM1 protein comprises a transmembrane domain from a rodent ICAM1 protein, optionally selected from a mouse ICAM1 protein or rat ICAM1 protein. In some embodiments, the ICAM1 protein comprises a transmembrane domain from a non-human primate ICAM1 protein, optionally selected from a monkey ICAM1 protein, chimpanzee ICAM1 protein, gorilla ICAM1 protein, orangutan ICAM1 protein, rhesus macaque ICAM1 protein, marmoset ICAM1 protein, or bonobo ICAM1 protein. In some embodiments, the ICAM1 protein comprises a transmembrane domain from an ungulate ICAM1 protein, optionally selected from a pig ICAM1 protein, horse ICAM1 protein, cattle ICAM1 protein, sheep ICAM1 protein, goat ICAM1 protein, or donkey ICAM1 protein.

A E-CAD cell anchor protein as described herein (e.g., as encoded by a nucleic acid or as present on a cell of a first mammalian species or cell of a second mammalian species), may be a complete E-CAD protein or a fragment thereof comprising an amino acid sequence that is a cell membrane anchor. In some embodiments, the E-CAD protein comprises a transmembrane domain from a human E-CAD protein. In some embodiments, the E-CAD protein comprises a transmembrane domain from a rodent E-CAD protein, optionally selected from a mouse E-CAD protein or rat E-CAD protein. In some embodiments, the CD protein comprises a transmembrane domain from a non-human primate E-CAD protein, optionally selected from a monkey E-CAD protein, chimpanzee E-CAD protein, gorilla E-CAD protein, orangutan E-CAD protein, rhesus macaque E-CAD protein, marmoset E-CAD protein, or bonobo E-CAD protein. In some embodiments, the E-CAD protein comprises a transmembrane domain from an ungulate E-CAD protein, optionally selected from a pig E-CAD protein, horse E-CAD protein, cattle E-CAD protein, sheep E-CAD protein, goat E-CAD protein, or donkey E-CAD protein.

A CLDN6 cell anchor protein as described herein (e.g., as encoded by a nucleic acid or as present on a cell of a first mammalian species or cell of a second mammalian species), may be a complete CLDN6 protein or a fragment thereof comprising an amino acid sequence that is a cell membrane anchor. In some embodiments, the CLDN6 protein comprises a transmembrane domain from a human CLDN6 protein. In some embodiments, the CLDN6 protein comprises a transmembrane domain from a rodent CLDN6 protein, optionally selected from a mouse CLDN6 protein or rat CLDN6 protein. In some embodiments, the CLDN6 protein comprises a transmembrane domain from a non-human primate CLDN6 protein, optionally selected from a monkey CLDN6 protein, chimpanzee CLDN6 protein, gorilla CLDN6 protein, orangutan CLDN6 protein, rhesus macaque CLDN6 protein, marmoset CLDN6 protein, or bonobo CLDN6 protein. In some embodiments, the CLDN6 protein comprises a transmembrane domain from an ungulate CLDN6 protein, optionally selected from a pig CLDN6 protein, horse CLDN6 protein, cattle CLDN6 protein, sheep CLDN6 protein, goat CLDN6 protein, or donkey CLDN6 protein.

A ITGB1 cell anchor protein as described herein (e.g., as encoded by a nucleic acid or as present on a cell of a first mammalian species or cell of a second mammalian species), may be a complete ITGB 1 protein or a fragment thereof comprising an amino acid sequence that is a cell membrane anchor. In some embodiments, the ITGB1 protein comprises a transmembrane domain from a human ITGB1 protein. In some embodiments, the ITGB1 protein comprises a transmembrane domain from a rodent ITGB1 protein, optionally selected from a mouse ITGB1 protein or rat ITGB1 protein. In some embodiments, the ITGB1 protein comprises a transmembrane domain from a non-human primate ITGB1 protein, optionally selected from a monkey ITGB1 protein, chimpanzee ITGB1 protein, gorilla ITGB1 protein, orangutan ITGB1 protein, rhesus macaque ITGB1 protein, marmoset ITGB 1 protein, or bonobo ITGB1 protein. In some embodiments, the ITGB1 protein comprises a transmembrane domain from an ungulate ITGB1 protein, optionally selected from a pig ITGB1 protein, horse ITGB1 protein, cattle ITGB1 protein, sheep ITGB1 protein, goat ITGB 1 protein, or donkey ITGB1 protein.

A ITGB2 cell anchor protein as described herein (e.g., as encoded by a nucleic acid or as present on a cell of a first mammalian species or cell of a second mammalian species), may be a complete ITGB2 protein or a fragment thereof comprising an amino acid sequence that is a cell membrane anchor. In some embodiments, the ITGB2 protein comprises a transmembrane domain from a human ITGB2 protein. In some embodiments, the ITGB2 protein comprises a transmembrane domain from a rodent ITGB2 protein, optionally selected from a mouse ITGB2 protein or rat ITGB2 protein. In some embodiments, the ITGB2 protein comprises a transmembrane domain from a non-human primate ITGB2 protein, optionally selected from a monkey ITGB2 protein, chimpanzee ITGB2 protein, gorilla ITGB2 protein, orangutan ITGB2 protein, rhesus macaque ITGB2 protein, marmoset ITGB2 protein, or bonobo ITGB2 protein. In some embodiments, the ITGB2 protein comprises a transmembrane domain from an ungulate ITGB2 protein, optionally selected from a pig ITGB2 protein, horse ITGB2 protein, cattle ITGB2 protein, sheep ITGB2 protein, goat ITGB2 protein, or donkey ITGB2 protein.

A JAM-B cell anchor protein as described herein (e.g., as encoded by a nucleic acid or as present on a cell of a first mammalian species or cell of a second mammalian species), may be a complete JAM-B protein or a fragment thereof comprising an amino acid sequence that is a cell membrane anchor. In some embodiments, the JAM-B protein comprises a transmembrane domain from a human JAM-B protein. In some embodiments, the JAM-B protein comprises a transmembrane domain from a rodent JAM-B protein, optionally selected from a mouse JAM-B protein or rat JAM-B protein. In some embodiments, the JAM-B protein comprises a transmembrane domain from a non-human primate JAM-B protein, optionally selected from a monkey JAM-B protein, chimpanzee JAM-B protein, gorilla JAM-B protein, orangutan JAM-B protein, rhesus macaque JAM-B protein, marmoset JAM-B protein, or bonobo JAM-B protein. In some embodiments, the JAM-B protein comprises a transmembrane domain from an ungulate JAM-B protein, optionally selected from a pig JAM-B protein, horse JAM-B protein, cattle JAM-B protein, sheep JAM-B protein, goat JAM-B protein, or donkey JAM-B protein.

A NCAM-1 cell anchor protein as described herein (e.g., as encoded by a nucleic acid or as present on a cell of a first mammalian species or cell of a second mammalian species), may be a complete NCAM-1 protein or a fragment thereof comprising an amino acid sequence that is a cell membrane anchor. In some embodiments, the NCAM-1 protein comprises a transmembrane domain from a human NCAM-1 protein. In some embodiments, the NCAM-1 protein comprises a transmembrane domain from a rodent NCAM-1 protein, optionally selected from a mouse NCAM-1 protein or rat NCAM-1 protein. In some embodiments, the NCAM-1 protein comprises a transmembrane domain from a non-human primate NCAM-1 protein, optionally selected from a monkey NCAM-1 protein, chimpanzee NCAM-1 protein, gorilla NCAM-1 protein, orangutan NCAM-1 protein, rhesus macaque NCAM-1 protein, marmoset NCAM-1 protein, or bonobo NCAM-1 protein. In some embodiments, the NCAM-1 protein comprises a transmembrane domain from an ungulate NCAM-1 protein, optionally selected from a pig NCAM-1 protein, horse NCAM-1 protein, cattle NCAM-1 protein, sheep NCAM-1 protein, goal NCAM-1 protein, or donkey NCAM-1 protein.

A MUC-4 cell anchor protein as described herein (e.g., as encoded by a nucleic acid or as present on a cell of a first mammalian species or cell of a second mammalian species), may be a complete MUC-4 protein or a fragment thereof comprising an amino acid sequence that is a cell membrane anchor. In some embodiments, the MUC-4 protein comprises a transmembrane domain from a human MUC-4 protein. In some embodiments, the MUC-4 protein comprises a transmembrane domain from a rodent MUC-4 protein, optionally selected from a mouse MUC-4 protein or rat MUC-4 protein. In some embodiments, the MUC-4 protein comprises a transmembrane domain from a non-human primate MUC-4 protein, optionally selected from a monkey MUC-4 protein, chimpanzee MUC-4 protein, gorilla MUC-4 protein, orangutan MUC-4 protein, rhesus macaque MUC-4 protein, marmoset MUC-4 protein, or bonobo MUC-4 protein. In some embodiments, the MUC-4 protein comprises a transmembrane domain from an ungulate MUC-4 protein, optionally selected from a pig MUC-4 protein, horse MUC-4 protein, cattle MUC-4 protein, sheep MUC-4 protein, goal MUC-4 protein, or donkey MUC-4 protein.

A PDGFRA cell anchor protein as described herein (e.g., as encoded by a nucleic acid or as present on a cell of a first mammalian species or cell of a second mammalian species), may be a complete PDGFRA protein or a fragment thereof comprising an amino acid sequence that is a cell membrane anchor. In some embodiments, the PDGFRA protein comprises a transmembrane domain from a human PDGFRA protein. In some embodiments, the PDGFRA protein comprises a transmembrane domain from a rodent PDGFRA protein, optionally selected from a mouse PDGFRA protein or rat PDGFRA protein. In some embodiments, the PDGFRA protein comprises a transmembrane domain from a non-human primate PDGFRA protein, optionally selected from a monkey PDGFRA protein, chimpanzee PDGFRA protein, gorilla PDGFRA protein, orangutan PDGFRA protein, rhesus macaque PDGFRA protein, marmoset PDGFRA protein, or bonobo PDGFRA protein. In some embodiments, the PDGFRA protein comprises a transmembrane domain from an ungulate PDGFRA protein, optionally selected from a pig PDGFRA protein, horse PDGFRA protein, cattle PDGFRA protein, sheep PDGFRA protein, goal PDGFRA protein, or donkey PDGFRA protein.

Amino acid sequences for CD8, ICAM1, E-Cad, CLDN6, ITGB1, ITGB2, JAM-B, NCAM-1, MUC-4, or PDGFRA of different species are available in public sequence databases, such as UniProt or NCBI, including annotation for transmembrane domains.

In some embodiments, the cell anchor protein is encoded by a component of a vector as described herein, such as disclosed in any one of SEQ ID NOS: 46-113, or a thereof having a percent identity to any one of SEQ ID NOS: 46-113 that conserves function of the cell anchor protein.

In some embodiments, the cell anchor protein comprises the amino acid sequence set forth in any one of SEQ ID NOS: 9-13, or an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with the sequence set forth in any one of SEQ ID NOS: 9-13.

### III. Organ, Organoid or Tissues

In an aspect, the current disclosure also encompasses an organ, an organoid, or a tissue, comprising a population of cells as described herein. The organ, organoid, or tissue develops from a population as described herein.

In an aspect, the organ, organoid or tissue comprises cells from a first mammalian species and a second mammalian species (e.g., a human and non-human species). In an aspect, the first or second mammalian may comprise less than 1% to less than 5%, or less than 5% to less than 10%, or less than 10% to less than 15%, or less than 15% to less than 20%, or less than 20% to less than 30%, or less than 30% to less than 40%, or less than 40% to less than 50%, or less than 50% to less than 60%, or less than 60% to less than 70%, or less than 70% to less than 80%, or less than 80% to less than 90%, or less that 90% to less than 91%, or less that 91% to less than 92%, or less that 92% to less than 93%, or less that 93% to less than 94%, or less that 94% to less than 95%, or less that 95% to less than 96%, or less that 96% to less than 97%, or less that 97% to less than 98%, or less that 98% to less than 99%, or less than 99% to less than 100%, of the cells of the organ, organoid or tissue, wherein the total of all mammal species cell types is 100%. In an aspect, the organ, organoid, or tissue comprises at least 80%, 81%, 82%, 83%, 84%, 85%, 86%, 87%, 88%, 89%, 90%, 91%, 92%, 93%, 94%, 95%, 96%, 97%, 98%, 99%, or more cells of a mammalian species for the target organ.

In an aspect, the organ, organoid or tissue may be selected from kidney, liver, heart, lung, pancreas, muscle (e.g., skeletal muscle), stomach, intestine, spleen, bladder, reproductive organs, bone marrow, or skin.

### IV. Methods of Manufacture

In an aspect, the current disclosure also encompasses a method of generating a mammalian organ, organoid, or tissue within an animal of another species. For example, generating a human organ, organoid, or tissue within an animal of a non-human species. The method may comprise: providing a population of one or more cell of a first mammalian species comprising an antigen-recognition domain at the cell membrane, and providing a cell of a second mammalian species comprising cells expressing an antigen at the cell membrane, whereby an interspecies chimera (e.g., chimeric embryo) is generated. The method may comprise: providing a population of one or more cell of a second mammalian species comprising an antigen-recognition domain at the cell membrane, and providing a cell of a first mammalian species comprising cells expressing an antigen at the cell membrane, whereby an interspecies chimera (e.g., chimeric embryo) is generated. In some embodiments the interspecies chimera is a chimeric embryo. In some embodiments the interspecies chimera is a chimeric blastocyst. The method may further comprise culturing the interspecies chimera under conditions that allow the interspecies chimera to develop into the organ, organoid, or tissue mass of the cell of a first mammalian species or the second mammalian species. In certain embodiments, the interspecies chimera is a human and non-human chimera.

The organ to be produced by the methods described herein may be any solid organ with a fixed shape, such as a kidney, liver, heart, lung, pancreas, muscle (e.g., skeletal muscle), stomach, intestine, spleen, bladder, reproductive organs, bone marrow, or skin. The organ may be produced in the body of a litter by developing totipotent cells or pluripotent cells within an embryonic cell that serves as a recipient. Totipotent cells or pluripotent cells can form organ types by being developed in an embryo. Accordingly, there is no limitation to the solid organ that can be produced depending on the specific type of cells used in the method.

A method of generating a mammalian organ, organoid, or tissue within another mammalian species may comprise microinjecting one or more stem cell from a first mammalian species into an embryo of the different species, thereby generating a chimeric embryo. The microinjection may comprise injecting the embryo of a host mammal with at least one stem cell of another mammal species, as described herein. A cell of a first mammalian species (e.g., an iPSC) may be injected into an embryo of a second mammalian species. For example, a human cell may be injected into an embryo of a rodent, non-human primate, or ungulate species. When the cells of the first and second mammalian species as described herein are engineered to comprise the antigen or antigen-binding protein, the interspecies chimera may have improved adherence which improves the chances of survival for the growth of the desired mammalian organ, organoid, or tissue, as well as the chances of survival of the host animal. The population of cells or interspecies chimera as described herein may be allogeneic or xenogeneic to the host cell and/or host animal.

In some aspects, a method of generating a mammalian organ, organoid, or tissue comprises creation of an interspecies chimera. Interspecies chimera may be generated by injection of one or more stem cell of a first mammalian species into an embryo of a second mammalian species. In some embodiments, about 1 to about 3 cells of a first mammalian species, inclusive, are injected into the one or more cell (e.g., embryo) of a second mammalian species. In some embodiments, about 4 to about 6 cells of a first mammalian species, inclusive, are injected into the one or more cell (e.g., embryo) of a second mammalian species. In some embodiments, about 7 to about 9 cells of a first mammalian species, inclusive, are injected into the one or more cell (e.g., embryo) of a second mammalian species. In some embodiments, about 10 to about 12 cells of a first mammalian species, inclusive, are injected into the one or more cell (e.g., embryo) of a second mammalian species. In some embodiments, about 13 to about 15 cells of a first mammalian species, inclusive, are injected into the one or more cell (e.g., embryo) of a second mammalian species. In some embodiments, about 16 to about 18 cells of a first mammalian species, inclusive, are injected into the one or more cell (e.g., embryo) of a second mammalian species. In some embodiments, about 19 to about 21 cells of a first mammalian species, inclusive, are injected into the one or cell (e.g., embryo) of a second mammalian species. In some aspects, once a chimeric embryo is produced it can be propagated for varying periods of time in culture, where it may undergo a series of developmental steps. Methods of culturing animal embryos (e.g., mammalian embryos) are known in the art. For some uses, the embryos can be brought to term, forming the chimeric animals having a mammalian organ, organoid, or tissue of a different species, as described herein.

The method may further comprise transferring the interspecies chimera into a pseudo-pregnant host mammal which may or may not be the same as the mammalian species from which the interspecies chimera blastocyst was prepared. The pseudo-pregnant host animal may be hormonally prepared. Progression of the development of the interspecies chimera may require implantation and placentation in the host mammal.

The host animal may be genetically modified, or comprise a gene knockout. In some embodiments, the host animal is knock of a gene involved in organogenesis for the human organ to be produced.

### V. Compositions

In some aspects, the current disclosure also encompasses pharmaceutical composition comprising a population of cells as described herein, and a pharmaceutically acceptable diluent(s), excipient(s), and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained.

In some aspects, pharmaceutical compositions herein may comprise stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof.

Techniques for formulation and administration of drugs may be found in “Remington's Pharmaceutical Sciences,” Mack Publishing Co., Easton, Pa., latest edition, which is incorporated herein by reference.

In certain aspects, pharmaceutical compositions disclosed herein may comprise agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, one or more of these agents may be added to improve the performance, efficacy, safety, shelf-life and/or other property of the population of cells of the present disclosure. In some aspects, additives may be biocompatible, without being harsh, abrasive, and/or allergenic.

### VI. Methods of Treatment

In some aspects, methods of treatment as disclosed herein may comprise administering to a subject in need thereof a human organ, organoid, or tissue as described herein. The human organ, organoid, or tissue may be manufactured in a non-human animal in accordance to the methods provided herein. The administration may be surgical administration.

A suitable subject may include may a human in need of replacement of an organ, organoid, or tissue. For example, the human subject may require replacement of a kidney, liver, heart, lung, pancreas, or muscle.

### VII. Sequences

In some aspects, any composition (e.g., a population of cells, organ, organoid, or tissue) or method as described herein may comprise the entirety of, or part of, any one of the sequences as described herein, or a sequence presented in the SEQUENCE TABLE presented below:

SEQUENCE TABLE

| SEQ | | | |
|---|---|---|---|
| ID | | | |
| NO | Type | Description | Sequence |
| 1 | Signal | Acrosin membrane signaling | MVEMLPTVAVLVLAVSVVAKDNTT |
| | | peptide (used for Nano-GPI | |
| | | constructs) | |
| 2 | Signal | CD8 membrane signaling | MASPLTRFLSLNLLLLGESIILGSGEA |
| | | peptide (used for Nano-CD8 | |
| | | constructs) | |
| 3 | Signal | Integrin membrane signaling | MNLQPIFWIGLISSVCCVFA |
| | | peptide (used for Nano-ITGB1 | |
| | | constructs) | |
| 4 | Signal | Cldn6 signal peptide (used for | MASTGLQILGIVLTLLGWVNA |
| | | Nano-Cldn6 constructs) | |
| 5 | Linker | Linker between membrane | LQEFATMQ |
| | | peptide and nanobody (used for | |
| | | Nano-GPI constructs) | |
| 6 | Linker | Linker between nanobody and | QLEN |
| | | GPI anchor signal (used for | |
| | | Nano-GPI constructs) | |
| 7 | Linker | Linker between Cldn6 signal | LVSCALPMWMWKVTAFIGN |
| | | peptide and nanobody (used for | |
| | | Nano-Cldn6 constructs) | |
| 8 | Lipid | GPI anchor (signal peptide from | GGISLLVQNTSWMLLLLLSLSLLQALDFISL |
| | Anchor | Thy-1 that marks protein for | |
| | | post-translational GPI addition) | |
| 9 | Anchor | CD8 anchor (transmembrane seq + | KPQAPELRIFPKKMDAELGQKVDLVCEVLGSVS |
| | Protein | c terminus) | QGCSWLFQNSSSKLPQPTFVVYMASSHNKITWD |
| | | | EKLNSSKLFSAMRDTNNKYVLTLNKFSKENEGY |
| | | | YFCSVISNSVMYFSSVVPVLQKVNSTTTKPVLRT |
| | | | PSPVHPTGTSQPQRPEDCRPRGSVKGTGLDFACD |
| | | | IYIWAPLAGICVALLLSLIITLICYH |
| 10 | Anchor | ICAM1 anchor (transmembrane | IVIITVVAAAVIMGTAGLSTYLYNRQRKIKKYRL |
| | Protein | seq) | QQAQKGTPMKPNTQATPP |
| 11 | Anchor | ITGB1 anchor (transmembrane | VENPECPTGPDIIPIVAGVVAGIVLIGLALLLIWKL |
| | Protein | seq) | LMIIHDRREFAKFEKEKMNAKWDTGENPIYKSA |
| | | | VTTVVNPKYEGK |
| 12 | Anchor | E-Cadherin anchor | SILGILGGILALLILILLLLLFLRRRAVVKEPLLPPE |
| | Protein | (transmembrane seq + c | DDTRDNVYYYDEEGGGEEDQDFDLSQLHRGLD |
| | | terminus) | ARPEVTRNDVAPTLMSVPRYLPRPANPDEIGNFI |
| | | | DENLKAADTDPTAPPYDSLLVFDYEGSGSEAASL |
| | | | SSLNSSESDKDQDYDYLNEWGNRFKKLADMYG |
| | | | GGEDD |
| 13 | Anchor | Cldn6 anchor (transmembrane | ALPQDLQAARALCVVTLLIVLLGLLVYLAGAKC |
| | Protein | seq + c terminus) | TTCVEDRNSKSRLVLISGIIFVISGVLTLIPVCWTA |
| | | | HSIIQDFYNPLVADAQKRELGASLYLGWAASGL |
| | | | LLLGGGLLCCACSSGGTQGPRHYMACYSTSVPH |
| | | | SRGPSEYPTKNYV |
| 14 | Antigen | wtGFP | MASKGEELFTGVVPILVELDGDVNGHKFSVSGE |
| | | | GEGDATYGKLTLKFICTTGKLPVPWPTLVTTFGY |
| | | | GVQCFARYPDHMKQHDFFKSAMPEGYVQERTIF |
| | | | FKDDGNYKTRAEVKFEGDTLVNRIELKGIDFKED |
| | | | GNILGHKLEYNYNSHNVYIMADKQKNGIKVNFK |
| | | | IRHNIEDGSVHLADHYQQNTPIGDGPVLLPDNHY |
| | | | LSTQSALSKDPNEKRDHMVLLEFVTAAGITHGM |
| | | | DELYKGLEVLFQGPSHHHHHH |
| 15 | Antigen | GFPuv | MSKGEELFTGVVPILVELDGDVNGHKFSVSGEG |
| | | | EGDATYGKLTLKFICTTGKLPVPWPTLVTTFSYG |
| | | | VQCFSRYPDHMKRHDFFKSAMPEGYVQERTISF |
| | | | KDDGNYKTRAEVKFEGDTLVNRIELKGIDFKED |
| | | | GNILGHKLEYNYNSHNVYITADKQKNGIKANFKI |
| | | | RHNIEDGSVQLADHYQQNTPIGDGPVLLPDNHY |
| | | | LSTQSALSKDPNEKRDHMVLLEFVTAAGILEHH |
| | | | HHHH |
| 16 | Antigen | GFPuv_M | MSKGEELFTGVVPILVELDGDVNGHKFSVSGEG |
| | | | EGDATYGKLTLKFICTTGKLPVPWPTLVTTFSYG |
| | | | VQCFSRYPDHMKRHDFFKSAMPEGYVQERTISF |
| | | | KDDGNYKTRAEVKFEGDTLVNRIELKGIDFKED |
| | | | GNILGHKLEYNYNSHNVYITADKQKNGIKANFKI |
| | | | RHNIEDGSVQLADHYQQNTPIGDGPVLLPDNHY |
| | | | LSTQSKLSKDPNEKRDHMVLLEFVTAAGILEHH |
| | | | HHHH |
| 17 | Nanobody | vhhGFP4 | VQLVESGGALVQPGGSLRLSCAASGFPVNRYSM |
| | | | RWYRQAPGKEREWVAGMSSAGDRSSYEDSVKG |
| | | | RFTISRDDARNTVYLQMNSLKPEDTAVYYCNVN |
| | | | VGFEYWGQGTQVTVSS |
| 18 | Nanobody | LaG-2 | MAQVQLVESGGGLVQAGGSLRLSCAASGRTFSN |
| | | | YAMGWFRQAPGKEREFVAAISWTGVSTYYADS |
| | | | VKGRFTISRDNDKNTVYVQMNSLIPEDTAIYYCA |
| | | | AVRARSFSDTYSRVNEYDYWGQGTQVTV |
| 19 | Nanobody | LaG-3 | MAQVQLVESGGGLVQAGGSLRVSCAASGRTYS |
| | | | DYAMGWFRQAPGKERDFVAGISGSGGDTYYAD |
| | | | SVKGRFTISRDNAKNTMYLQMNSLKPEDTAVYF |
| | | | CAARTGTVLFTSRVDYRYWGQGTQVTV |
| 20 | Nanobody | LaG-5 | QVQLVESGGGLVQAGGSLRLSCAASGSIFSSNA |
| | | | MAWYRQTPEKQRELICDITRGGITKCADSVKGRF |
| | | | TISRDNTKNTVYLQMNSLKSEDTAVYYCAAKSE |
| | | | GYFGFPRVENEYPYWGQGTQVTV |
| 21 | Nanobody | LaG-6 | MAQVQLVESGGGLVQAGGSLRLSCAASGRTFST |
| | | | SAMAWFRQAPGKEREFAAGITWISSSTYYTDSV |
| | | | KGRFTISRDNAKNTVYLQMNSLKPEDTAVYYCA |
| | | | AKSEGYFGFPRVENEYPYWGQGTQVTV |
| 22 | Nanobody | LaG-8 | MAQVQLVESGGGLVQHGGSLRLSCVTSGFTFDI |
| | | | HDMGWFRQAPGKERDIVARISKSGDITYYADSV |
| | | | KGRFIISRDNTKNTVYLQMNSLKPEDTAVYYCA |
| | | | ATLRATITSFDEYVYRGQGTQVTVS |
| 23 | Nanobody | LaG-9 | MADVQLVESGGGLVQAGGSLRLSCAASGRTFST |
| | | | SAMGWFRQAPGKEREFVARITWSAGYTAYSDSV |
| | | | KGRFTISRDKAKNTVYLQMNSLKPEDTAVYYCA |
| | | | SRSAGYSSSLTRREDYAYWGQGTQVTVS |
| 24 | Nanobody | LaG-10 | MAQVQLVESGGGLVQAGDSLQLSCAFSGGTFST |
| | | | YAMGWFRQAPGKEREFVGGISRSGATTNYEDSV |
| | | | KGRFTISKDNTKNTVYLQLNSLKPEDTAVYYCA |
| | | | ARNNILPVTTIDKYEYWGQGTQVTV |
| 25 | Nanobody | LaG-11 | MADVQLVESGGRSVRAGDSLRLSCLASGGTFSL |
| | | | YAMGWFRQAPGKEREFVAAVTWSGGSTYYTDS |
| | | | VKGRFSISRDNAKNTVYLQMNSLKPEDTAVYYC |
| | | | AVRTSGFFGSIPVTERAFDYWGQGTQVTVS |
| 26 | Nanobody | LaG-12 | MASGAAGGGLGEGLVQAGGSLRLSCAASGRTF |
| | | | NSYPMAWFRQAPGKEREFVAALGWSGGSTDYA |
| | | | DSVKGRFTISRDNSKNTVYLEMNSLKPDDTGVY |
| | | | YCALRRRGGVYNTYSGEKDYDYWGQGTQVTVS |
| 27 | Nanobody | LaG-17 | MAQVQLVESGGGLVQAGGSLRLSCAASGRTYSI |
| | | | SAMGWFRQAPGKEREFVAGISRSGGTTYYADPV |
| | | | KGRFTISRDNAKNTVYLQMNSLKPEDTAVYYCA |
| | | | ARARGWTTFPAREIEYDYWGQGTQVTV |
| 28 | Nanobody | LaG-18 | MAQVQLVESGGGLVQTGGSLKLSCTASVRTLSY |
| | | | YHVGWFRQAPGKEREFVAGIHRSGESTFYADSV |
| | | | KGRFTISRDNAKNTVHLQMNSLKPEDTAVYYCA |
| | | | QRVRGFFGPLRSTPSWYDYWGQGTQVTVS |
| 29 | Nanobody | LaG-43 | MADVQLVESGGGLVQPGGSLRLSCEASGGAFST |
| | | | VAMGWFRQAPGKEREFVGAITWTAGSTYYADS |
| | | | AKGRFTISRDNAKNTVHLQMNSLKPEDTAVYYC |
| | | | AQRVRGFFGPLRTTPSWYEYWGQGTQVTVS |
| 30 | Nanobody | LaG-19 | MAQVQLVESGGGLVQAGGSLRLSCAASGPTGA |
| | | | MAWFRQAPGKEREFVGGISRSGTDTYYVDSVKG |
| | | | RFTIDRDNAKNTVYLQMNSLKPEDTAVYYCAAR |
| | | | RSQILFTSRTDYEFWGQGTQVTV |
| 31 | Nanobody | LaG-21 | MAQVQLVESGGGLVQAGGSLRLSCAASGPTGA |
| | | | MAWFRQAPGMEREFVGGISGSETDTYYADFVK |
| | | | GRLTVDRDNVKNTVDLQMNSLKPEDTAVYYCA |
| | | | ARRRVTLFTSRADYDFWGQGTQVTVS |
| 32 | Nanobody | LaG-42 | MADVQLVESGGGLVQAGDSLRLSCAASGPTGA |
| | | | MAWFHQGLGKEREFVGGISPSGDNIYYADSVKG |
| | | | RFTIDRDNAKNTVSLQMNSLKPEDMGVYYCAA |
| | | | RRRVTLFTSRTDYEFWGRGTQVTVS |
| 33 | Nanobody | LaG-24 | MADVQLVESGGGLVQPGGSLRLSCAASGEIASII |
| | | | AIGWYRQAPGKQRESVALITRSGMITYGDSAQG |
| | | | RFTISRDDAKNTVYLHMDDLVPEDTAVYYCNAK |
| | | | KVSFGDYWGQGTQVTVS |
| 34 | Nanobody | LaG-26 | MAQVQLVESGGGLVQAGASMRLSCAASGITFSL |
| | | | YHWVWFRQAAGREHEFVAGIIRSGGETLSADSV |
| | | | KDRFIISRDDAKNTLYLQMNMLQPEDTATYYCA |
| | | | ATHRADWYSSAFREYIFRGQGTQVTVS |
| 35 | Nanobody | LaG-27 | MADVQLVESGGGLVQAGGSLRLSCTASGLTIST |
| | | | YNIGWFRQAPGKEREFVGIIIRNGDTTYYADSVK |
| | | | GRFTISRDNAKNTVYLQMNSVKPADAAVYSCGA |
| | | | TVRAGAAAEQYNSYIFRGQGTQVTV |
| 36 | Nanobody | LaG-29 | MAQVQLVESGGGLVQAGAALRLSCAASGGTFSF |
| | | | YNMGWFRQAPGKEREFVVSISRSGGGTAYADSV |
| | | | KGRFTISRDNAKNTAYLQMNSLKPEDTAVYYCA |
| | | | AGLRDWGREGEPHYWGQGTQVTVS |
| 37 | Nanobody | LaG-30 | MAQVQLVESGGGLVQAGGSLRLSCAASGRTFST |
| | | | SAMGWFRQAPGREREFVAAITWTVGNTIYGDSM |
| | | | KGRFTISRDRTKNTVDLQMDSLKPEDTAVYYCT |
| | | | ARSRGFVLSDLRSVDSFDYKGQGTQVTVS |
| 38 | Nanobody | LaG-35 | MADVQLVESGGGLVQAGGSLRLSCTVSGRTFSN |
| | | | YAMGWFRQAPGKEREFVAGISWTGGHTLYTDS |
| | | | VKGRFTISRDNAKNTVYLQMNSLKPEDTALYYC |
| | | | AADRAADFFAQRDEYDYWGQGTQVTVS |
| 39 | Nanobody | LaG-37 | MAQVQFVESGGGTVQDGDFLRLSCTASGDTFSN |
| | | | YHAGWFRQPPGREREFVAAISWTGEGTLYADSV |
| | | | KGQFTISRDNAKNAMYLQMNRLKPEDTAVYYC |
| | | | AAARSVGFTWRSSKSNDYAYWGQGTQVTV |
| 40 | Nanobody | LaG-41 | MADVQLVESGGGLVQAGGSLRLSCAASGPTGA |
| | | | MAWFRQAPGKEREFVGGISGSETDTYYVDSVKG |
| | | | RFTVDRDNVKNTVYLQMNSLKPEDTAVYYCAA |
| | | | RRRITLFTSRTDYDFWGRGTQVTV |
| 41 | Nanobody | LaM-2 | MAQVQLVESGGGLVQAGGSLRLSCATSGFTFSD |
| | | | YAMGWFRQAPGKEREFVAAISWSGHVTDYADS |
| | | | VKGRFTISRDNVKNTVYLQMNSLKPEDTAVYSC |
| | | | AAAKSGTWWYQRSENDFGSWGQGTQVTVSKE |
| | | | AI |
| 42 | Nanobody | LaM-3 | MAQVQLVQSGGGLVQAGGSLRLSCAASGRTFSD |
| | | | IAVGWFRQTPGKEREFVAAISWSGLIINYGDSVE |
| | | | DRFTISRDNAKSAVYLQMNSLKPEDTAVYYCAA |
| | | | RIGMNYYYAREIEYPYWGQGTQVTVSKCY |
| 43 | Nanobody | LaM-4 | MAQVQLVESGGSLVQPGGSLRLSCAASGRFAES |
| | | | SSMGWFRQAPGKEREFVAAISWSGGATNYADSA |
| | | | KGRFTLSRDNTKNTVYLQMNSLKPDDTAVYYC |
| | | | AANLGNYISSNQRLYGYWGQGTQVTVSSPFT |
| 44 | Nanobody | LaM-6 | MAQVQLVESGGGLVQAGGSLRLSCVASGSAPSF |
| | | | FAMAWYRQSPGNERELVAALSSLGSTNYADSVK |
| | | | GRFTISMDNAKNTVYLQMNNVNAEDTAVYYCA |
| | | | AGDFHSCYARKSCDYWGQGTQVTVS |
| 45 | Nanobody | LaM-8 | MAQVQLVESGGGLVQAGGSLRLSCAVSGRPFSE |
| | | | YNLGWFRQAPGKEREFVARIRSSGTTVYTDSVK |
| | | | GRFSASRDNAKNMGYLQLNSLEPEDTAVYYCA |
| | | | MSRVDTDSPAFYDYWGQGTQVTVSTPRS |
| 46 | Vector | PB_TRE3G Promoter_GFP | See Sequence Listing |
| | | Nano_hICAM1_HA_EF1a | |
| | | Promoter_Tet-on | |
| | | 3G_PuroR_AmpR | |
| 47 | Vector | PB_TRE3G Promoter_Nano | See Sequence Listing |
| | | LAG5 | |
| | | (Kd = 14,200 nM)_hICAM1_HA | |
| | | EF1a Promoter_Tet-on | |
| | | 3G_PuroR_AmpR | |
| 48 | Vector | PB_TRE3G Promoter_Nano | See Sequence Listing |
| | | LAG6 | |
| | | (Kd = 310 nM)_hICAM1_HA_EF1a | |
| | | Promoter_Tet-on | |
| | | 3G_PuroR_AmpR | |
| 49 | Vector | PB_TRE3G Promoter_Nano | See Sequence Listing |
| | | LAG8(Kd = 20,000 nM)_hICAM1_ | |
| | | HA_EF1a Promoter_Tet-on | |
| | | 3G_PuroR_AmpR | |
| 50 | Vector | PB_TRE3G Promoter_Nano | See Sequence Listing |
| | | LAG10(Kd = 97nM)_hICAM1_HA_ | |
| | | EF1a Promoter_Tet-on | |
| | | 3G_PuroR_AmpR | |
| 51 | Vector | PB_TRE3G Promoter_Nano | See Sequence Listing |
| | | LAG11(Kd = 22,900 nM)_hICAM1_ | |
| | | HA_EF1a Promoter_Tet-on | |
| | | 3G_PuroR_AmpR | |
| 52 | Vector | PB_TRE3G Promoter_Nano | See Sequence Listing |
| | | LAG17 | |
| | | (Kd = 50 nM)_hICAM1_HA_EF1a | |
| | | Promoter_Tet-on | |
| | | 3G_PuroR _mpR | |
| 53 | Vector | PB_TRE3G Promoter_Nano | See Sequence Listing |
| | | LAG18 | |
| | | (Kd = 3,800 nM)_hICAMI_HA_ | |
| | | EF1a Promoter_Tet-on | |
| | | 3G_PuroR_AmpR | |
| 54 | Vector | PB_TRE3G Promoter_Nano | See Sequence Listing |
| | | LAG27(Kd = 9.5 nM)_hICAM1_ | |
| | | HA_EF1a Promoter_Tet-on | |
| | | 3G_PuroR_AmpR | |
| 55 | Vector | PB_TRE3G Promoter_Nano | See Sequence Listing |
| | | LAG29 | |
| | | (Kd = 110 nM)_hICAM1_HA_EF1a | |
| | | Promoter_Tet-on | |
| | | 3G_PuroR_AmpR | |
| 56 | Vector | PB_TRE3G Promoter_Nano | See Sequence Listing |
| | | LAG30 | |
| | | (Kd = 0.5 nM)_hICAM1_HA_EF1a | |
| | | Promoter_Tet-on | |
| | | 3G_PuroR_AmpR | |
| 57 | Vector | PB_TRE3G Promoter_Nano | See Sequence Listing |
| | | LAG42 | |
| | | (Kd = 600 nM)_hICAM1_HA_ | |
| | | EF1a Promoter_Tet-on | |
| | | 3G_PuroR_AmpR | |
| 58 | Vector | PB_CAG | See Sequence Listing |
| | | Promoter_EGFP_hICAM1_Amp | |
| | | R | |
| 59 | Vector | PB_Primed Oct4 | See Sequence Listing |
| | | Promoter_EGFP_hICAM1_Amp | |
| | | R | |
| 60 | Vector | cag-pb-itr-v2-gfp-cd8 | See Sequence Listing |
| 61 | Vector | cag-pb-itr-v2-gfp-gpi | See Sequence Listing |
| 62 | Vector | cag-pb-itr-v2-nuc-mko | See Sequence Listing |
| 63 | Vector | cag-pb-itr-v2-nuc-mko-nano-cd8 | See Sequence Listing |
| 64 | Vector | cag-pb-itr-v2-nuc-mko-nano-gpi | See Sequence Listing |
| 65 | Vector | cag-pb-itr-v2 | See Sequence Listing |
| 66 | Vector | cag-pb-itr-v2-nano-cd8 | See Sequence Listing |
| 67 | Vector | cag-pb-itr-v2-nano-gpi-chromo- | See Sequence Listing |
| | | version | |
| 68 | Vector | gfp-cd8-constitutive | See Sequence Listing |
| 69 | Vector | gfp-gpi-puro | See Sequence Listing |
| 70 | Vector | gfp-morphotrap-cd8 | See Sequence Listing |
| 71 | Vector | nanobody-gpi-puro | See Sequence Listing |
| 72 | Vector | nano-cd8-constitutive | See Sequence Listing |
| 73 | Vector | pds0226-real-version | See Sequence Listing |
| 74 | Vector | pds0227-real-version | See Sequence Listing |
| 75 | Vector | pds0229-real-version | See Sequence Listing |
| 76 | Vector | cag-pb-itr-v2-gfp-ecad-in-frame- | See Sequence Listing |
| | | 5-25-22 | |
| 77 | Vector | cag-pb-itr-v2-gfp-ecad-new- | See Sequence Listing |
| | | version-522 | |
| 78 | Vector | cag-pb-itr-v2-nano-ecad-new- | See Sequence Listing |
| | | version-522 | |
| 79 | Vector | gfp-cldn6-hybrid-construct | See Sequence Listing |
| 80 | Vector | gfp-e-cadherin-c-terminus | See Sequence Listing |
| 81 | Vector | gfp-mecad-in-frame-n-term | See Sequence Listing |
| 82 | Vector | nanobody-cldn6-hybrid- | See Sequence Listing |
| | | construct | |
| 83 | Vector | nanobody-e-cadherin-c-hybrid | See Sequence Listing |
| 84 | Vector | nano-mecad-in-frame-n-term | See Sequence Listing |
| 85 | Vector | cag-itr-piggybac-plasmid | See Sequence Listing |
| 86 | Vector | cag-pb-itr-v2-gfp-itgb1 | See Sequence Listing |
| 87 | Vector | cag-pb-itr-v2-mcherry-cd8 | See Sequence Listing |
| 88 | Vector | cag-pb-itr-v2-mcherry- | See Sequence Listing |
| | | cytoplasmic | |
| 89 | Vector | cag-pb-itr-v2-mcherry-gpi | See Sequence Listing |
| 90 | Vector | cag-pb-itr-v2-mcherry-nano- | See Sequence Listing |
| | | itgb1 | |
| 91 | Vector | cag-pb-itr-v2-mcherry-nuclear | See Sequence Listing |
| 92 | Vector | cag-pb-itr-v2-nuc-mcherry- | See Sequence Listing |
| | | nano-gpi | |
| 93 | Vector | gfp-c6-and-nanobody-c6-same- | See Sequence Listing |
| | | plasmid | |
| 94 | Vector | gfp-cd8-ki-v2 | See Sequence Listing |
| 95 | Vector | gfp-cd8-ki-v3 | See Sequence Listing |
| 96 | Vector | gfp-cd8-knock-in | See Sequence Listing |
| 97 | Vector | gfp-cldn6-hybrid-construct- | See Sequence Listing |
| | | short-version | |
| 98 | Vector | gfp-pdgfr-in-morphotrap-vector | See Sequence Listing |
| 99 | Vector | human-nano-cd8-ki | See Sequence Listing |
| 100 | Vector | human-nano-gpi-chromo-ki | See Sequence Listing |
| 101 | Vector | mouse-rosa26-gfp-cd8 | See Sequence Listing |
| 102 | Vector | mouse-rosa26-gfp-gpi | See Sequence Listing |
| 103 | Vector | mouse-rosa26-gfp-ki | See Sequence Listing |
| 104 | Vector | mouse-rosa26-nano-cd8 | See Sequence Listing |
| 105 | Vector | mouse-rosa26-nano-mko-gpi | See Sequence Listing |
| 106 | Vector | nanobody-cldn6-hybrid- | See Sequence Listing |
| | | construct-short-version | |
| 107 | Vector | nanobody-linker-gpi-puro | See Sequence Listing |
| 108 | Vector | nanobody-pdgfr-in-morphotrap- | See Sequence Listing |
| | | vector | |
| 109 | Vector | nano-cd8-ki-v2 | See Sequence Listing |
| 110 | Vector | nano-cd8-ki-v3 | See Sequence Listing |
| 111 | Vector | nano-gpi-chromo-ki-constitutive | See Sequence Listing |
| 112 | Vector | nano-gpi-ki-mcherry-fusion | See Sequence Listing |
| 113 | Vector | nano-mecad | See Sequence Listing |

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1: Incompatibility in Cell Adhesion Constitutes a Barrier to Interspecies Chimerism

Interspecies blastocyst complementation holds great potential to address the global shortage of human organs for transplant by growing human tissues and organs in animals. However, a major challenge in this approach is the limited chimerism of human cells in evolutionarily distant animal hosts due to a myriad of xenogeneic barriers. One key obstacle is the disparity in cell adhesion, as human cells struggle to form adhesive junctions with animal cells, both in culture and when introduced into mouse embryos. To overcome this barrier, a synthetic biology strategy was developed that leverages nanobody-antigen interactions to artificially enhance cell adhesion. Cells were engineered to express nanobodies and their corresponding antigens in the outer plasma membrane, which significantly improved the bonding between different species' PSCs during in vitro cell adhesion assays and increased the chimerism of human PSCs in mouse embryos. Manipulation of interspecies pluripotent cell adhesion provides valuable insights into the dynamics of cell interaction in chimera formation and early embryonic development.

Blastocyst complementation is a strategy to address the worldwide shortage of donor organs. This technique involves using pluripotent stem cells (PSCs) to complement an organogenesis-disabled host embryo, leading to donor cell enrichment in the emptied developmental organ niche (Zhen et al., Development (2021), 148). Initially developed for studying gene function within the same species (Chen et al., Proc Natl Acad Sci (1993), 90:4528-4532), blastocyst complementation has recently been adopted for interspecies organogenesis, which involves transplanting donor PSCs from one species into the embryo of another species to grow a specific organ. Although successful in generating functional tissues like the pancreas (Kobayashi et al., Cell (2010), 142:787-799; Yamaguchi et al., Nature (2017), 542:191-196; Wu et al., Cell (2017), 168:473-486), kidney (Goto et al., Nat Commun (2019), 10:451), and forebrain (Huang et al., bioRxiv (2023)) between closely related rats and mice, applying this technique to more evolutionarily distant species has faced significant challenges due to various xenogeneic barriers.

Some of these barriers have been previously characterized, including cell competition and developmental timing. Cell competition was recently identified as a barrier to interspecies chimerism, where mouse PSCs become “winners” and induce apoptosis in the “loser” human PSCs upon cell-cell contact (Zheng et al., Nature (2021), 592:272-276). Regarding developmental timing, human PSCs differentiate at a slower rate than mouse PSCs and maintain their species-specific developmental pace during teratoma formation within a mouse host (Barry et al., Dev Biol (2017), 423:101-110). Consequently, they may struggle to keep up with the rapid pace of differentiation during mouse embryogenesis. However, a recent study showed that the differentiation of hPSCs is somewhat accelerated during co-differentiation with mouse PSCs (Brown et al., PLOS Comput Biol (2021), 17). Other potential components of the xenogeneic barrier that have not yet been investigated in detail include mismatches between ligands and receptors from different species and incompatibility in cell adhesion. Cell adhesion plays a pivotal role in tissue and organ formation during development (Halbleib and Nelson, Genes Dev (2006), 20:3199-3214), and its disruption is associated with various developmental disorders (Petruzzelli et al., Am J Med (1999), 106:467-476). Differential cell adhesion due to mismatches in cell adhesion molecules (CAMs) may hinder the effective integration of donor PSCs from an evolutionarily distant species with host epiblast cells, thereby inhibiting their chimeric contribution to host embryos.

### Human Cells Perish in Chimeric Blastocysts Cultured In Vitro

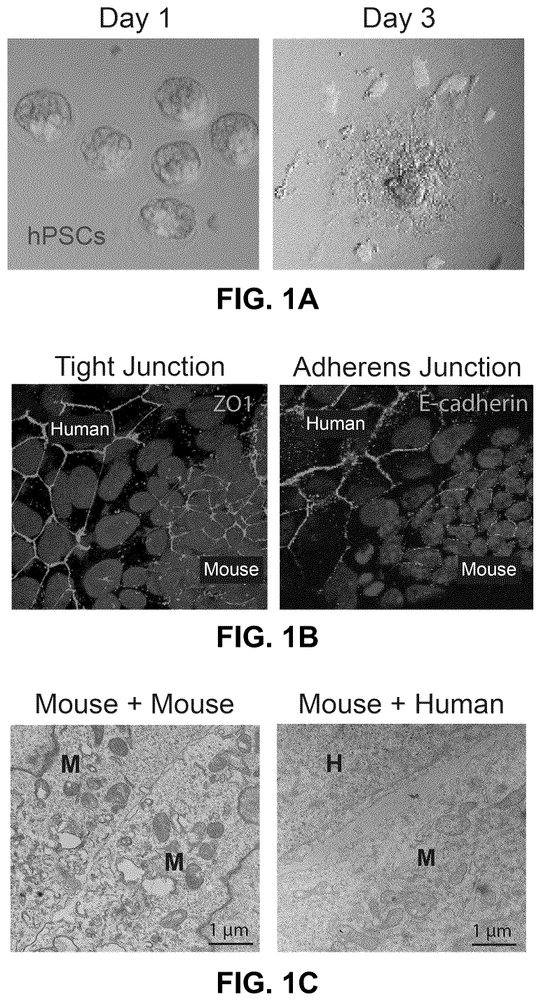

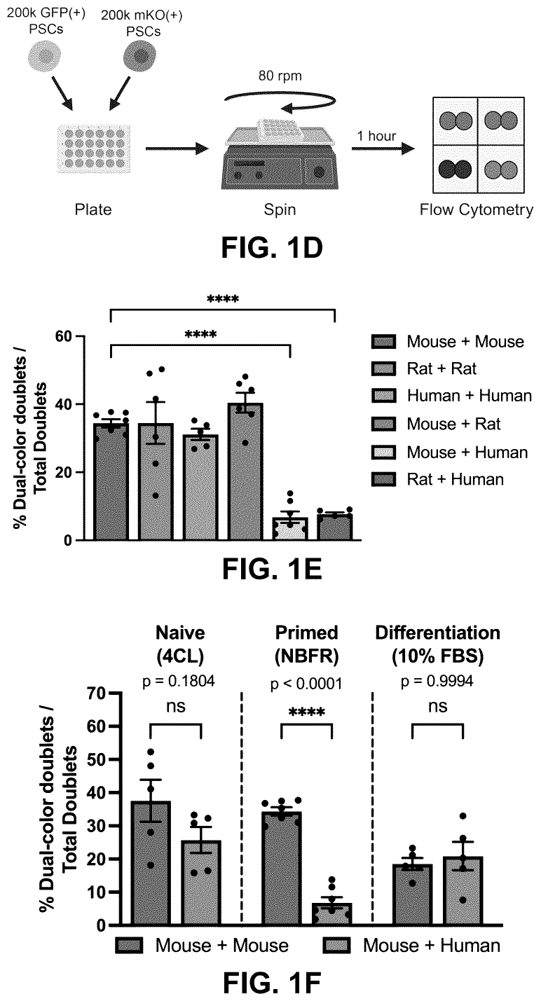

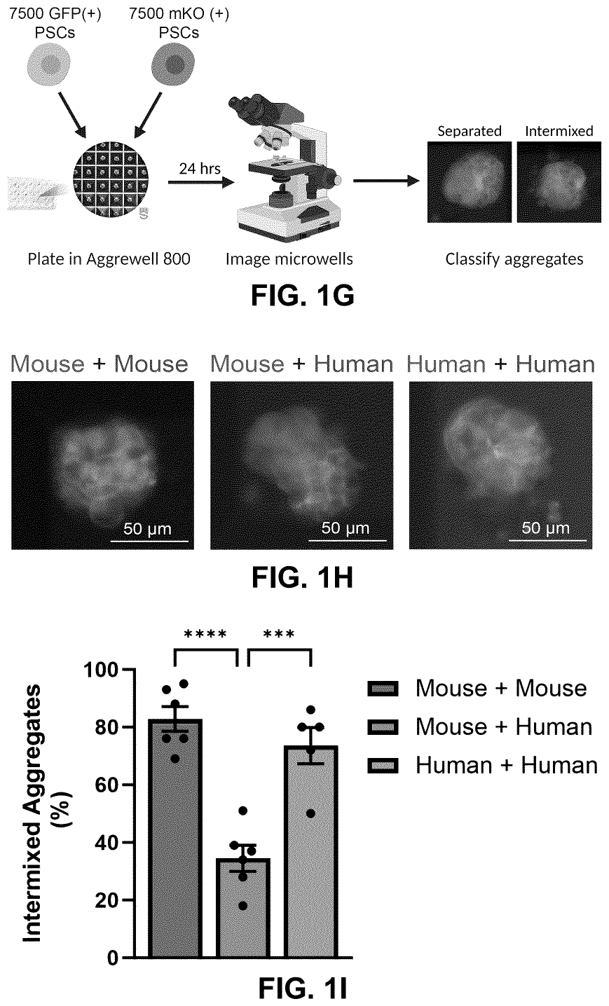

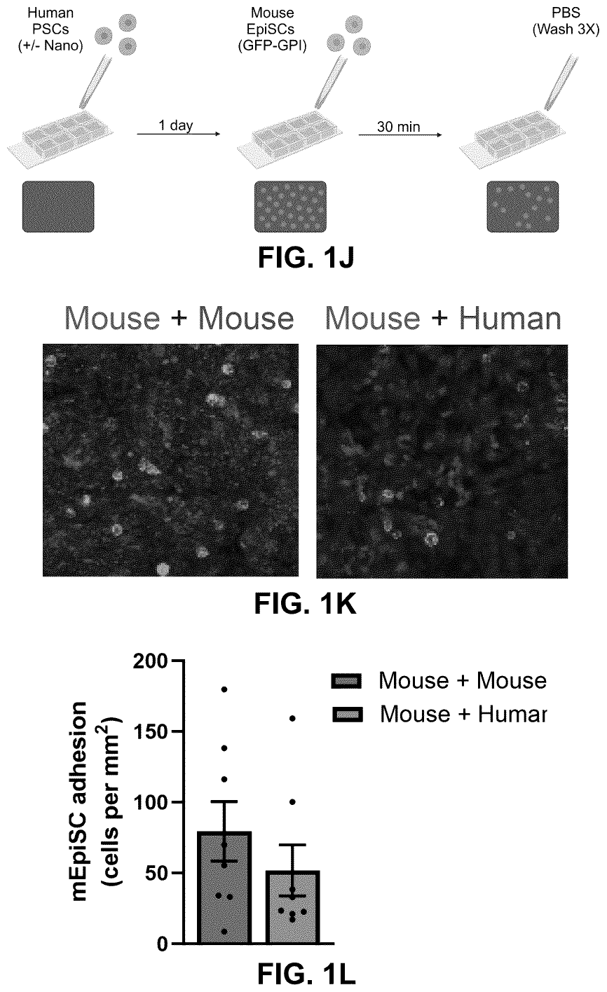

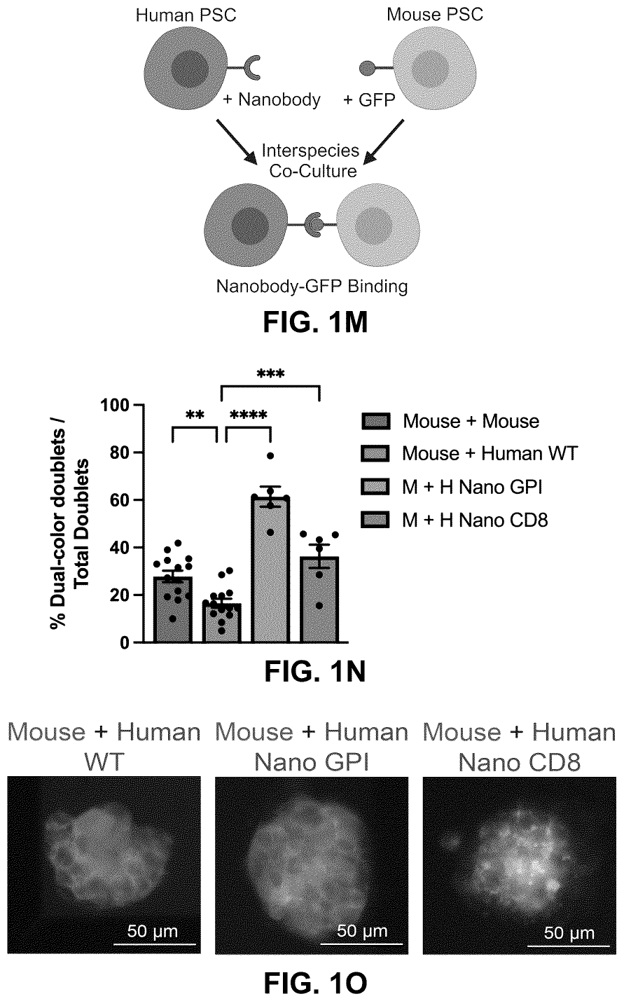

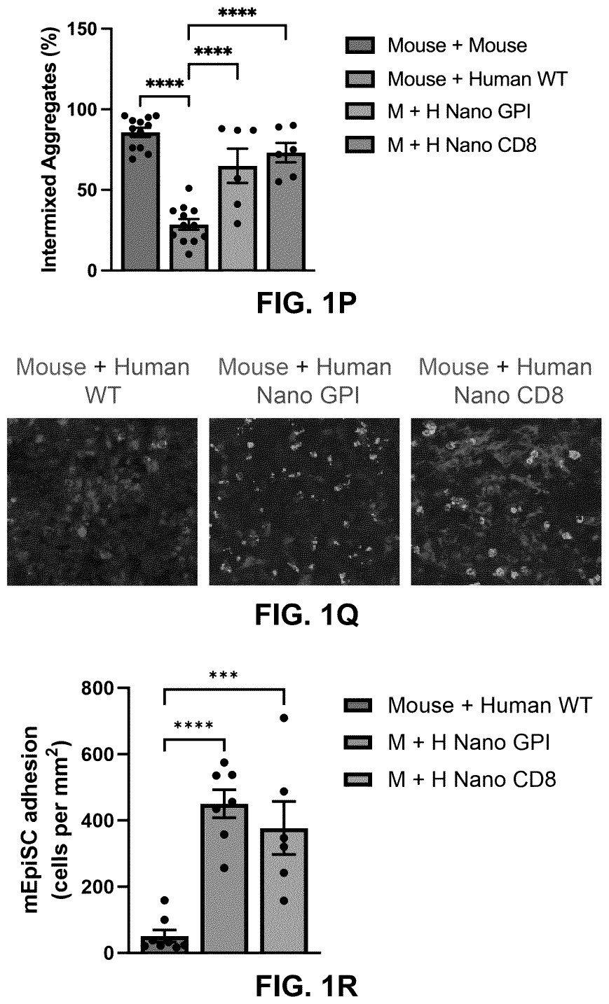

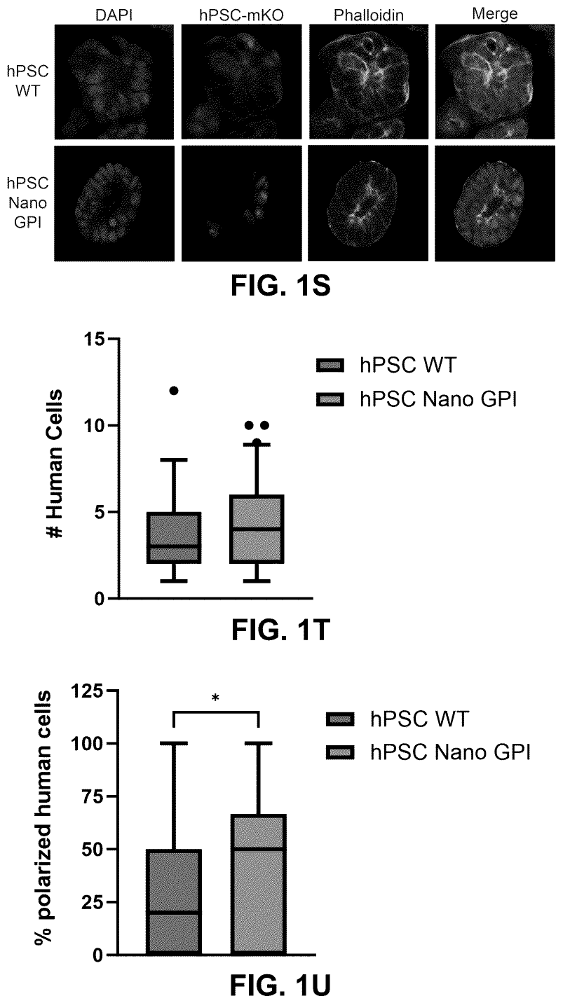

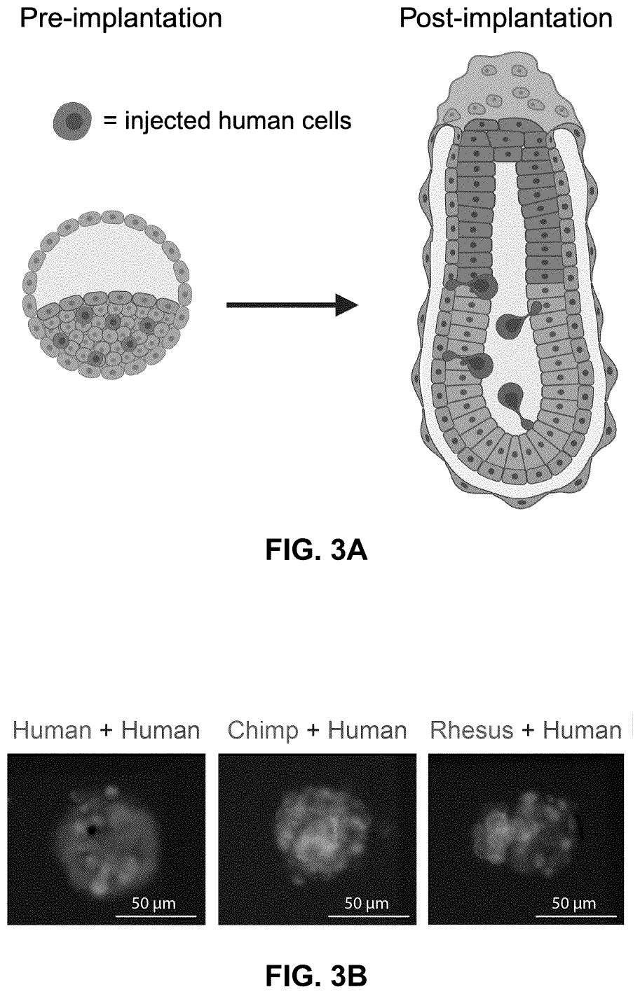

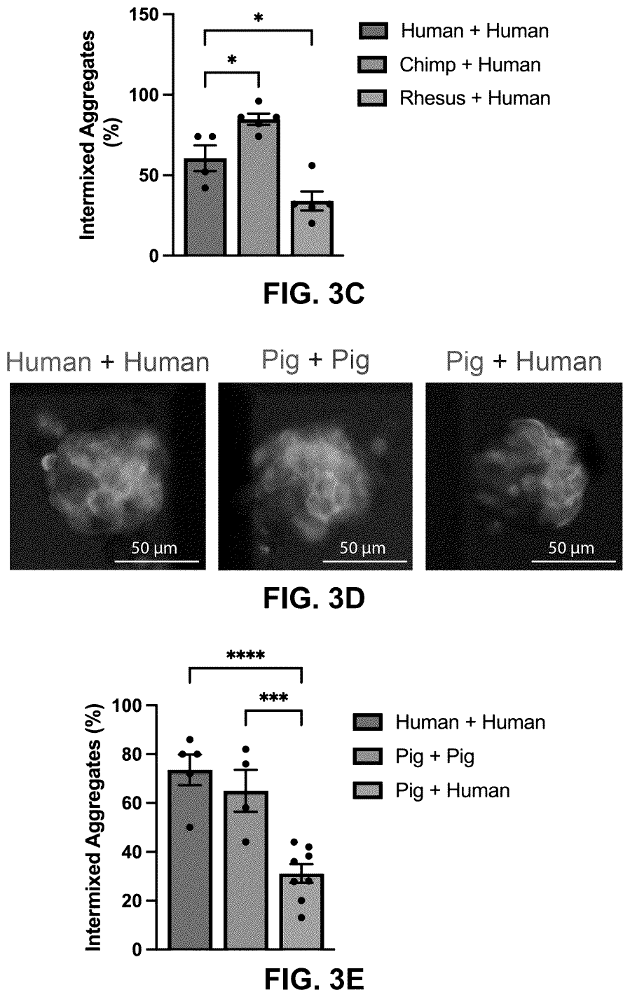

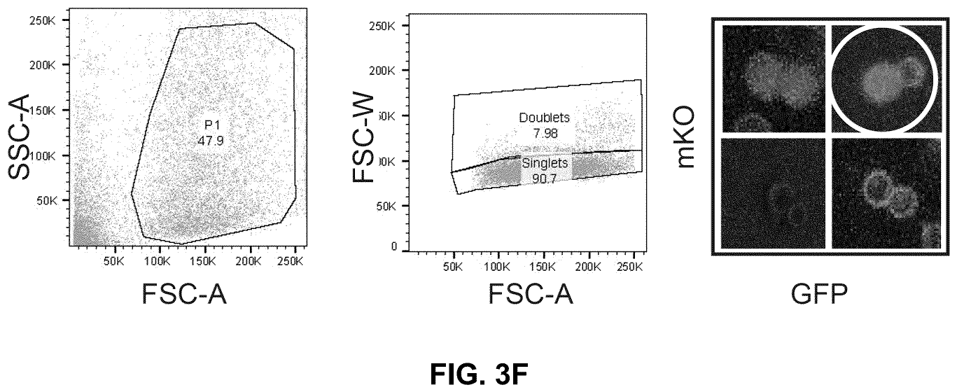

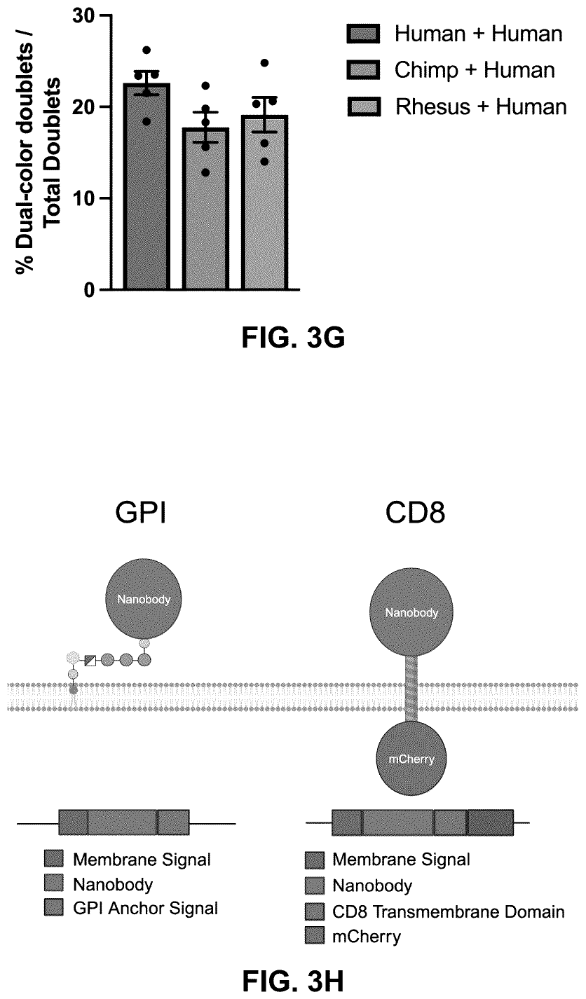

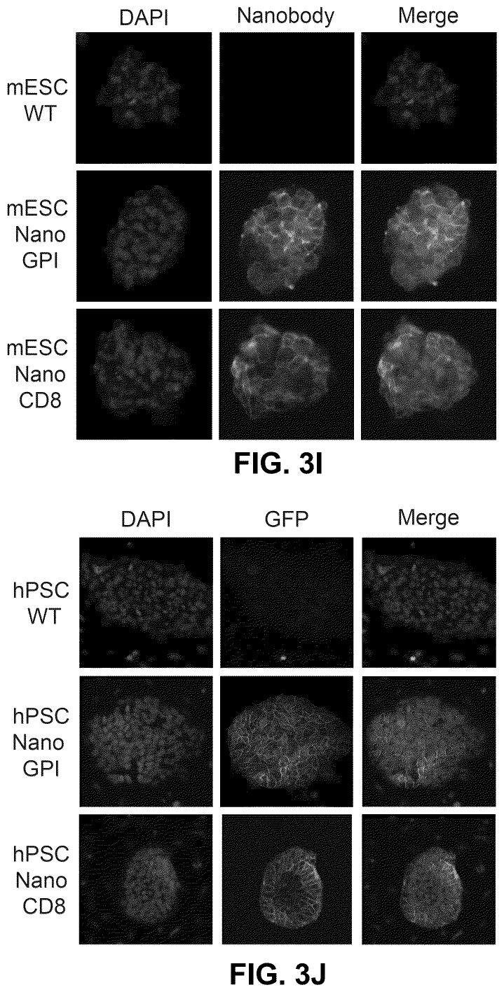

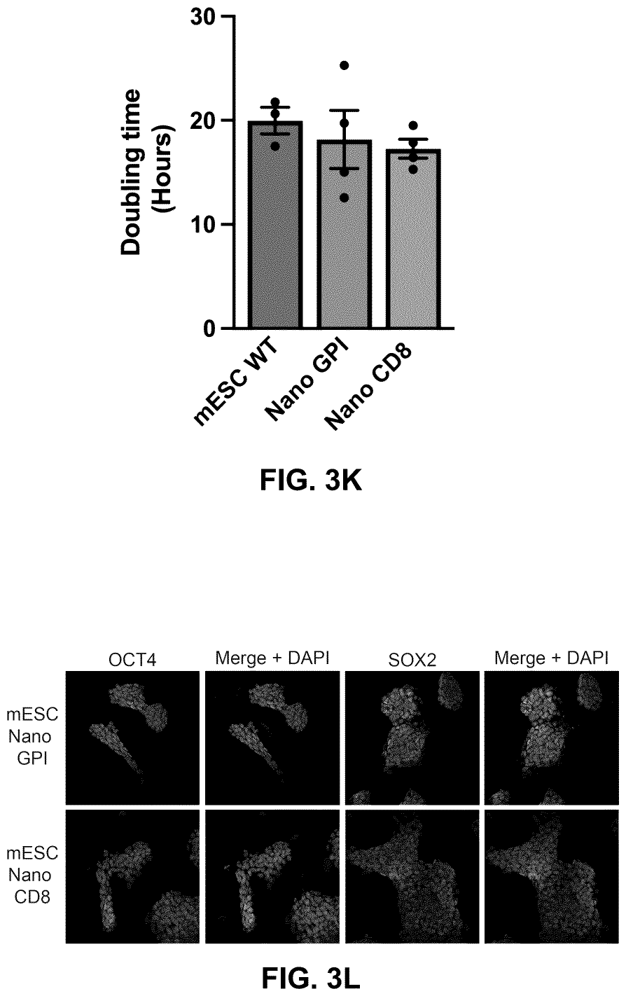

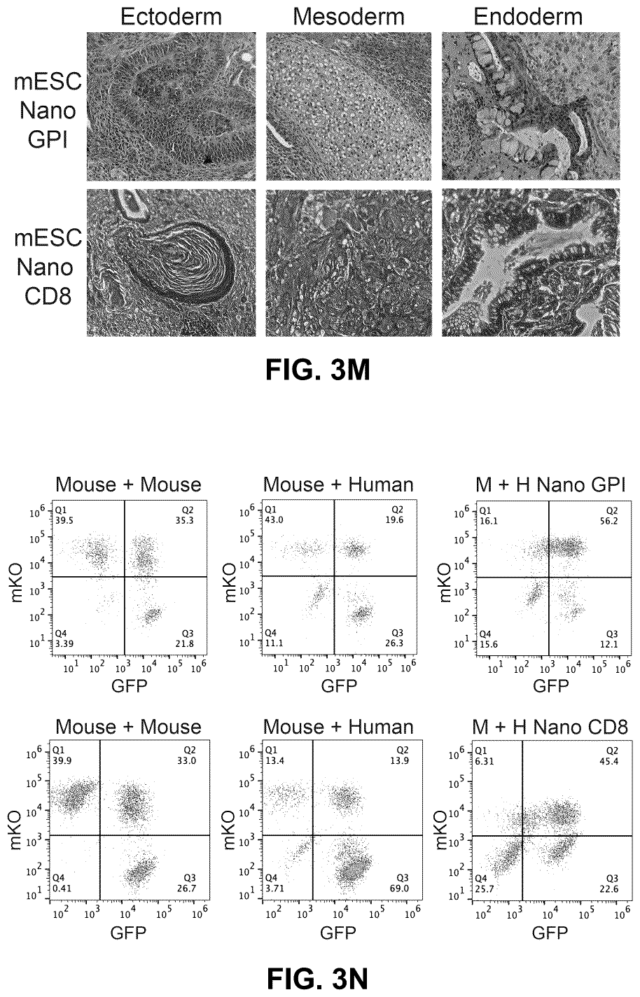

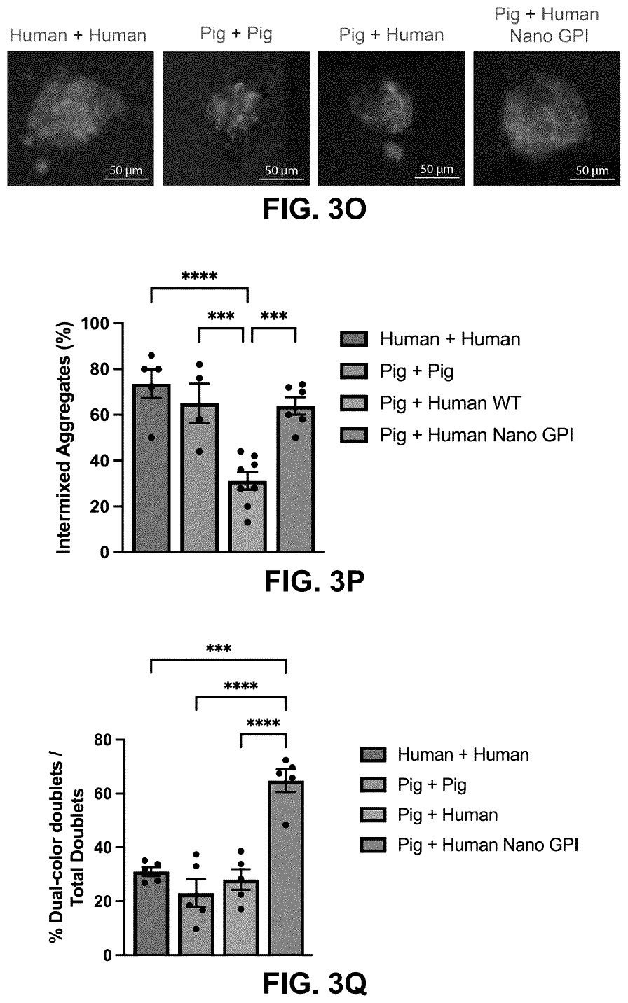

Experiments were conducted to study cell adhesion barriers. Mouse blastocysts were cultured in vitro following the injection of human induced pluripotent stem cells (hiPSCs). In many instances, the human cells perished within the first three days post-injection, likely due to cell competition, a form of “quality control” during primed pluripotency (Zheng et al., Nature (2021), 592:272-276; Baker, Nat Rev Genet (2020), 21:683-697). Remarkably, when human cells did survive, they predominantly localized at the periphery of the embryo, rather than within the epiblast (FIG. 1A). During the transition from naïve to primed pluripotency, epiblast cells undergo mesenchymal-to-epithelial transition (MET) (Sheng, Dev Biol (2015), 401:17-24) and are held together by strong anchoring junctions, such as tight and adherens junctions. This extrusion of human cells from the embryo may be attributed to their inability to form adhesive junctions with mouse epiblast cells, which would impede their participation in subsequent developmental processes (FIG. 3A).

### Adhesion of Human Cells in Co-Cultures

To study the cell adhesion barrier in greater detail, PSCs from different species were co-cultured. Staining was performed on mouse epiblast stem cells (mEpiSCs) (Wu et al., Nature (2015), 521:316-321) and hiPSCs (Wu et al., Cell (2017), 168:473-486) co-cultured in NBFR medium (which maintains cells in the primed state of pluripotency) for markers of tight junctions (ZO1) and adherens junctions (E-cadherin). The junctions were found to predominantly form between cells of the same species, whereas junction formation was significantly less frequent at the interfaces between mouse and human cells (FIG. 1B). Further analysis was conducted using correlative light-electron microscopy (CLEM) to observe the co-cultured mEpiSCs and hiPSCs at higher magnification. The results showed that cells of the same species maintained direct contact along substantial portions of their membranes. In contrast, interactions between mouse and human cells were characterized by numerous gaps, with only limited areas of direct membrane contact (FIG. 1C).

To quantify interspecies PSC adhesion, a modified doublet flow cytometry assay (Segal and Stephany, Cytometry (1984), 5:169-181; Burshtyn and Davidson, Methods Mol Biol (2010), 612:89-96) was adapted for use in testing. This assay measures the percentage of cell pairs (doublets) composed of both a red and a green cell, which can originate from the same or different species (FIGS. 1D and 3F). Results from this assay revealed that rodent (mouse/rat) EpiSCs and primate (human, chimpanzee and rhesus macaque) PSCs showed poor adhesion to one another, while higher percentages of dual-colored doublets were obtained from cells of the same species or from closely related species, such as mouse and rat, human and chimpanzee, or human and rhesus monkey

(FIGS. 1E and 3G). These initial studies were performed in primed cells, and then the doublet assay was performed using naïve PSCs cultured in the 4CL condition (Mazid et al., Nature (2022), 605:315-324) and randomly differentiating PSCs after one week of culture in serum-containing medium. It was discovered that the interspecies adhesion incompatibility was most pronounced in primed PSCs and less evident in naïve PSCs. Intriguingly, after a week of differentiation, no notable differences in adhesion were observed between mouse-mouse and mouse-human pairs (FIG. 1F).

These findings of an interspecies adhesion barrier were further supported by other cell adhesion assays, including the 3D co-aggregation assay and the wash assay. The 3D co-aggregation assay is based on the differential adhesion hypothesis, which suggest that cells with strong mutual adhesion will mix uniformly, while those with weaker adhesion tendencies will form separate clusters or layers (Steinberg, Science (1963), 141:401-408; Foty and Steinberg, Dev Biol (2005), 278:255-263). Similar assays have been used previously to examine adhesion between neural stem cells at different stages of differentiation (Karpowicz et al., J Neurosci (2007), 27:5437-5447), and between cells isolated from different germ layers in zebrafish embryos (Schotz et al., HFSP J (2008), 2:42-56). The 3D co-aggregation assay was performed on several combinations of rodent, primate, and ungulate species. The results showed that co-cultured PSCs from the same species or closely related species, such as mouse and rat or human and chimpanzee, tended to distribute themselves evenly, resulting in high percentages of fully intermixed aggregates. However, PSCs from more evolutionarily distant species, like human and mouse or human and pig, tended to remain segregated (FIGS. 1H-1I, 3B-3E). Interestingly, aggregates of human and rhesus monkey PSCs also remained predominantly segregated, despite both being primate species. This may be attributed to the evolutionary distance separating these two species, approximately 28.8 million years ago (MYA), compared to ˜13.1 MYA between mouse and rat or ˜6.4 MYA between human and chimpanzee (Steppan et al., Syst Biol (2004), 53:533-553; Rhesus Macaque Genome et al., Science (2007) 316, 222-234).

### Development of a Nanobody Based Adhesion System

The wash assay, which has previously been used to measure cell adhesion to substrates like protein-coated surfaces or cell monolayers (Butler et al., Methods Mol Biol (2009). 467:211-228; Chen et al., Cancer Res (2009), 69:3713-3720), was adapted for use as a cell-adhesion assay. This assay quantified the number of mEpiSCs expressing membrane-localized GFP (Rhee et al., Genesis (2006), 44:202-218) anchored by glycosylphosphatidylinositol [GPI] (GFP-GPI mEpiSCs) that remained adhered to a confluent monolayer of red fluorescent mEpiSCs or hiPSCs. The assay involved plating GFP-GPI mEpiSCs on top of the red fluorescent mEpiSC/hiPSC monolayer and allowing a brief 30-minute incubation period for cell attachment. The wells were then washed with PBS to remove any unattached cells and imaged using a fluorescent microscope to quantify the remaining GFP-GPI mEpiSCs (FIG. 1J). The results showed that only a limited number of GFP-GPI mEpiSCs adhered to the hiPSC monolayer, while a larger number adhered to the mEpiSC monolayer (FIG. 1K-1L).

Cell adhesion incompatibility between PSCs of different species may occur because of structural and expression-level variations in CAMs. Modifying CAMs of hPSCs to match those of the host species was proposed as a solution, but it is impractical due to the diverse and dynamic nature of CAM expression during development. Instead, a synthetic biology approach utilizing membrane-tethered nanobody-antigen interactions to enhance cell adhesion. nanobodies, derived from camelid antibodies, are single-domain fragments known for their versatile expression and ability to bind small antigens (Beghein and Gettemans, Front Immunol (2017), 8:771). In this method, cells from one species express a membrane-bound nanobody, while cells from another species display the corresponding membrane-bound antigen, facilitating strong nanobody-antigen interactions upon contact (FIG. 1M). This strategy was originally developed to induce artificial cell adhesion in bacterial systems and has recently been extended to mammalian models (Glass and Riedel-Kruse, Cell (2018), 174:649-658; Stevens et al., Nature (2023), 614:144-152). However, applying this synthetic biology approach to enhance adhesion between cells from different species (e.g., a human cell and a non-human cell), and to improve chimeric contribution of hPSCs in animal embryos remains an unmet need.

To this end, naïve mouse embryonic stem cells (mESCs), primed mEpiSCs, H9 human embryonic stem cells (hESCs) (Thomson et al., Science (1998), 282:1145-1147), and human foreskin fibroblast (HFF)-iPSCs (Wu et al., Cell (2017), 168:473-486) were engineered to express the high-affinity vhhGFP4 anti-GFP nanobody (Kubala et al., Protein Sci (2010), 19:2389-2401) on their outer surface. Different methods were utilized to anchor the nanobody to the plasma membrane, including a peripheral GPI anchor (Rhee et al., Genesis (2006), 44:202-218) in hiPSCs and the transmembrane domain from the CD8 protein (Harmansa et al., Elife (2017), 6) in hESCs (FIG. 3H). The membrane localization and the ability of both the GPI- and CD8-anchored anti-GFP nanobodies to bind purified GFP protein was confirmed (FIGS. 31-3J). Importantly, expressing these membrane-anchored anti-GFP nanobodies did not compromise the pluripotency and self-renewal capabilities of the PSCs (FIGS. 3K-3M).

Using the doublet flow cytometry assay, it was observed that hPSCs expressing anti-GFP nanobodies exhibited significantly higher adhesion rates to GFP-GPI mEpiSCs compared to wild-type (WT) hPSCs. Remarkably, these adhesion rates were on par with, or even exceeded, those observed in same-species cell interactions (FIGS. 1N and 3N).

### In-Vitro Testing of the Nanobody Based Adhesion System

The functioning of the nanobodies in facilitating cell-adhesion was next tested via in-vitro assay systems. In the 3D co-aggregation assay, replacing WT hiPSCs with Nano-GPI hiPSCs or Nano-CD8 hESCs significantly increased the percentage of fully intermixed aggregates (FIGS. 10-1P). These levels were comparable to aggregates formed from PSCs of a single species. The Nano-GPI was observed to have no effect on cell morphology, and the Nano-CD8 induced slight membrane blebbing and deformation (data not shown). In the wash assay, a considerable number of GFP-GPI mEpiSCs remained attached after washing when the underlying monolayer consisted of either Nano-GPI or Nano-CD8 hiPSCs, compared to WT hiPSCs (FIGS. 1Q-1R). Significantly, the adherence level of GFP-GPI mEpiSCs to a monolayer of Nano-GPI hiPSCs or Nano-CD8 hESCs was markedly higher than when these cells were plated onto a monolayer of mEpiSCs (FIGS. 1L and 1R). Additionally, the nanobody system increased adhesion between human and pig cells, as demonstrated by the doublet and co-aggregation assays (FIGS. 3O-3Q).

To better replicate the conditions of a peri-implantation chimeric embryo, mEpiSCs and hiPSCs were co-cultured in Aggrewell™ 800 microwell plates, overlaying them with a 10% Matrigel extracellular matrix to promote cell polarization and the development of rosette and lumen structures within the cell aggregates (Bedzhov, Zernicka-Goetz, Cell (2014), 156:1032-1044). Rosettes typically appeared after 24 hours of culture, and lumens began to form after 48 hours. At this stage, the aggregates were fixed and stained with phalloidin to highlight the apical actin ring around the lumen surface. In these chimeric aggregates composed of both mEpiSCs and hiPSCs, analysis was performed on the extent of human cell polarization and contribution to lumen formation, defined by an elongated shape and direct contact with the central lumen. The findings revealed that Nano-GPI hiPSCs were significantly more effective at contributing to lumen formation than WT hiPSCs (FIGS. 1S-1U).

In-vivo testing of the nanobody based adhesion system

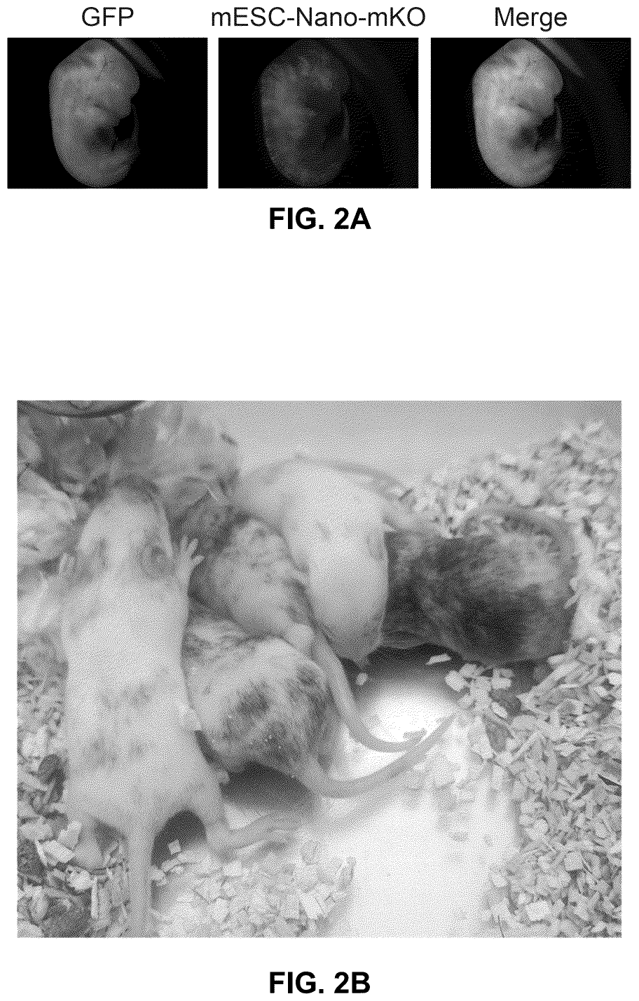

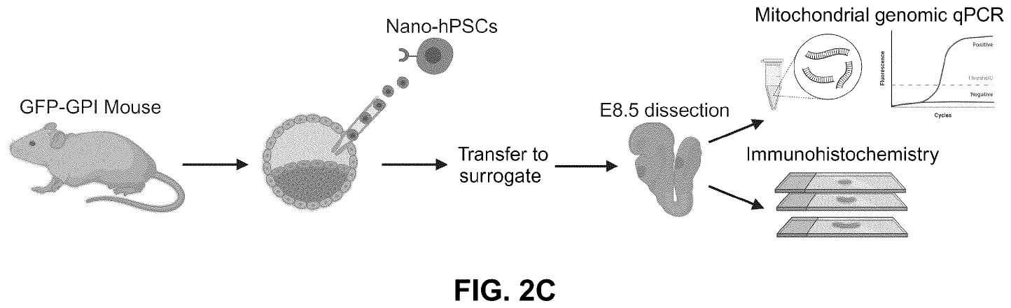

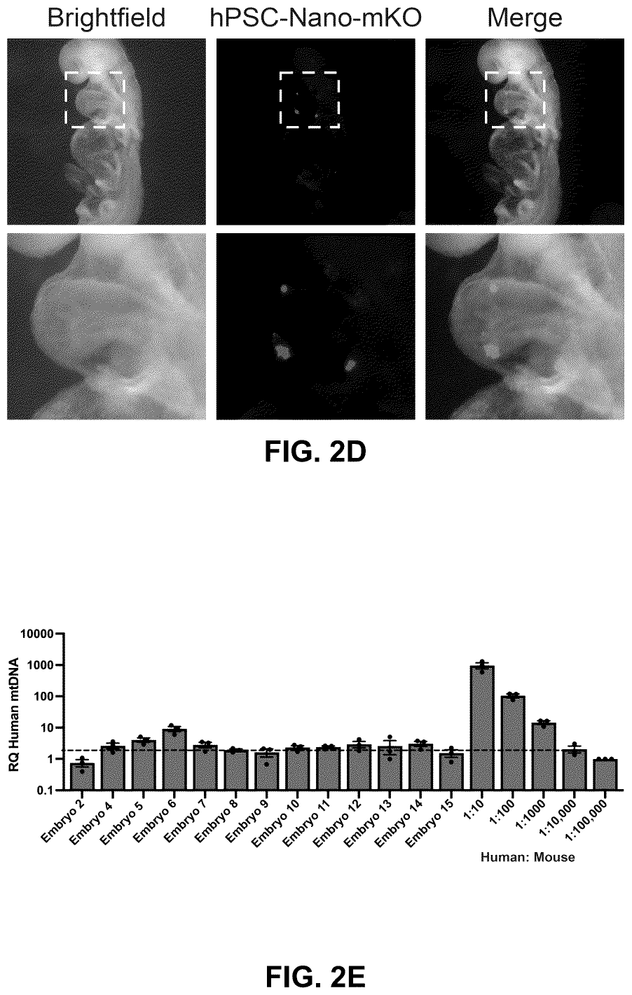

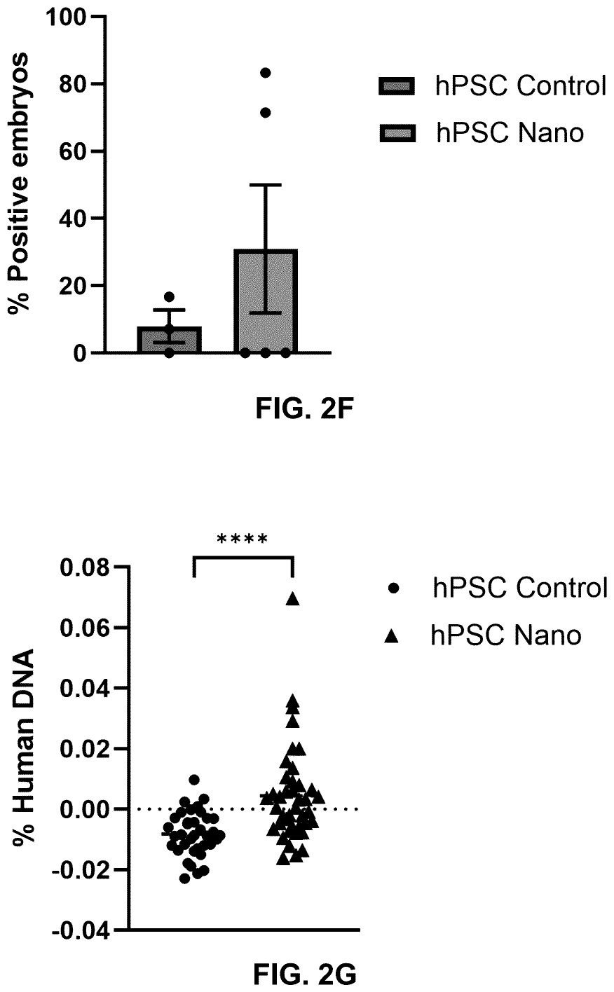

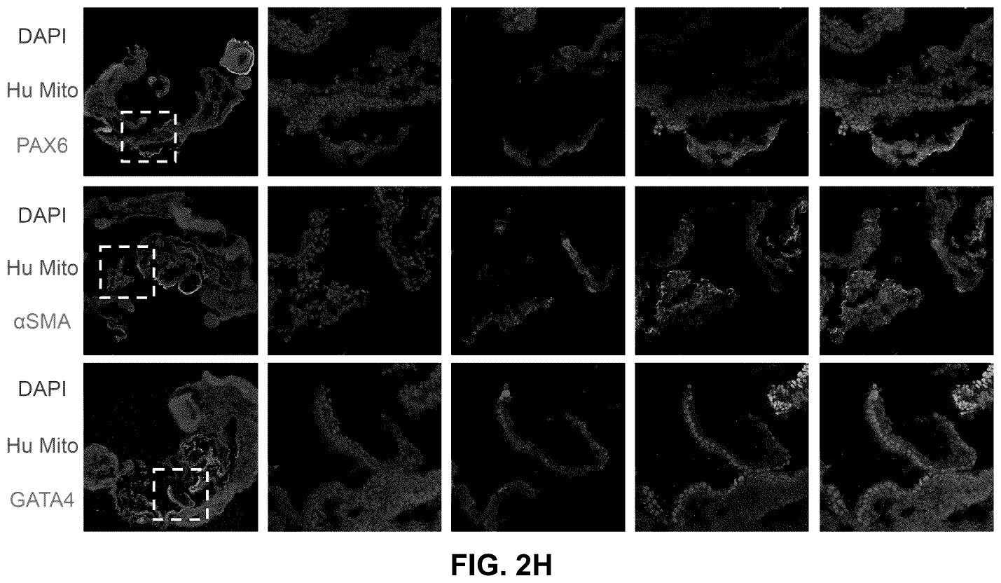

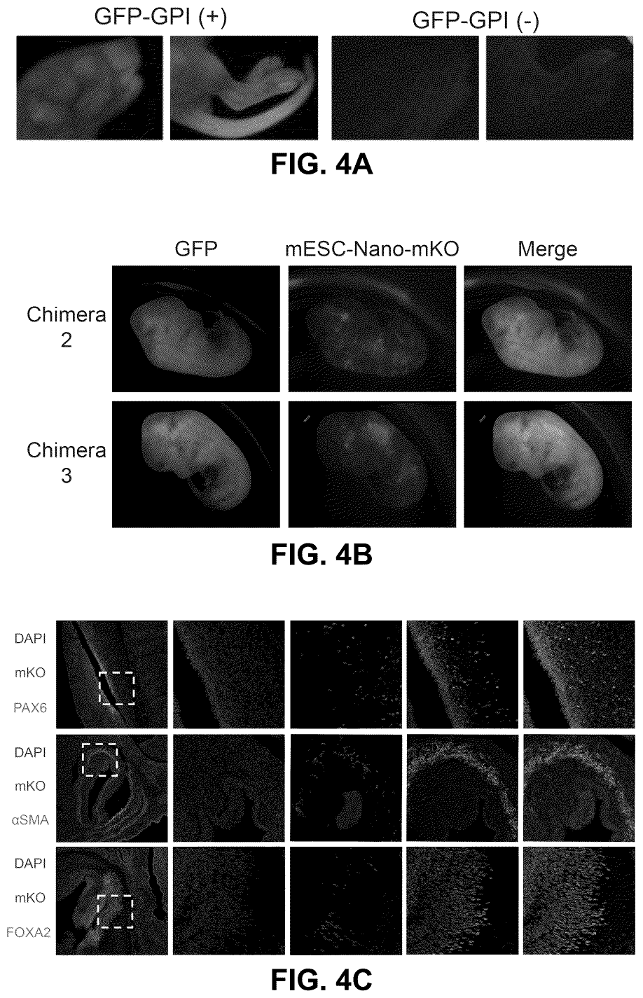

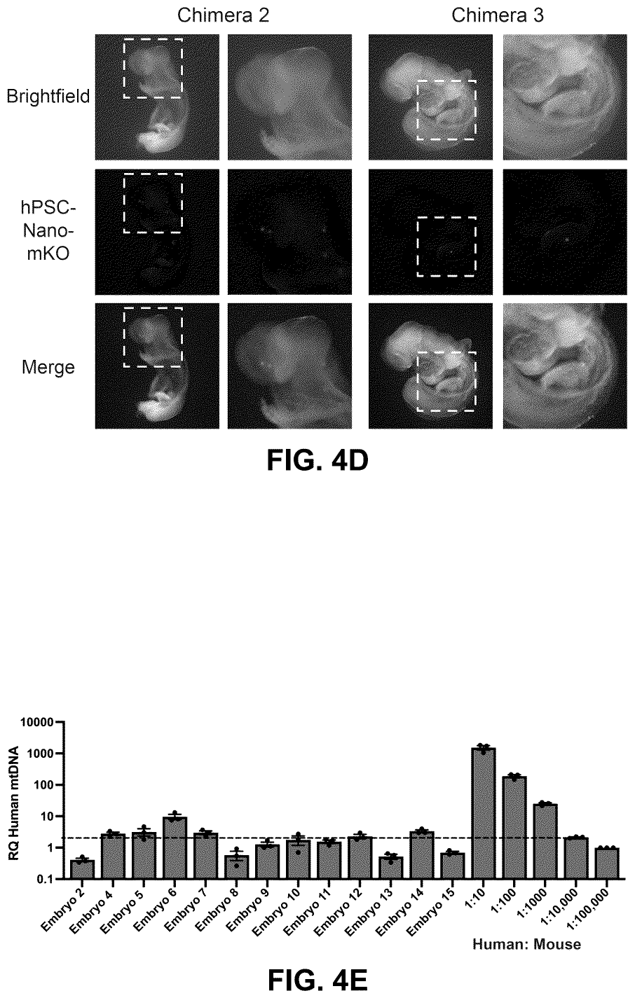

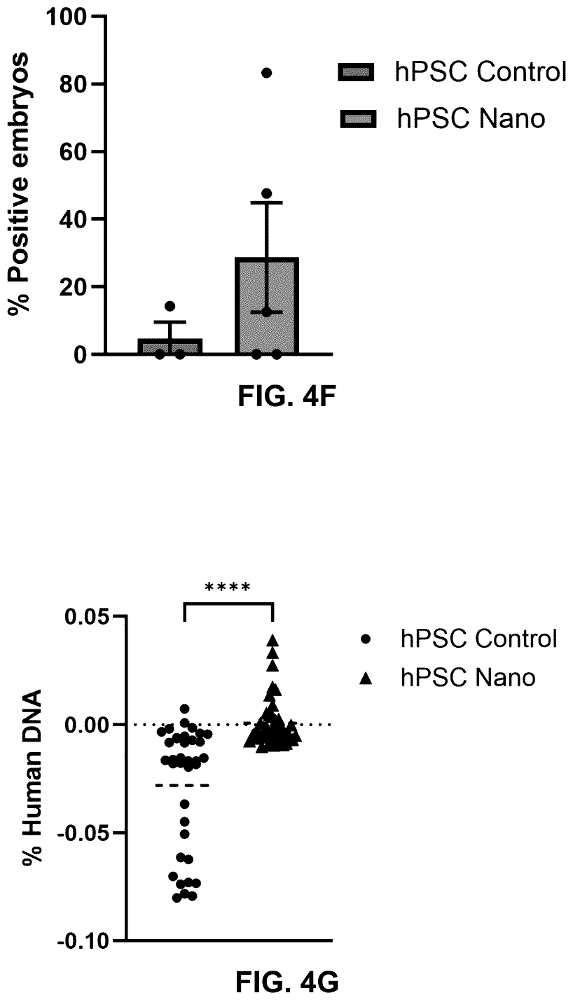

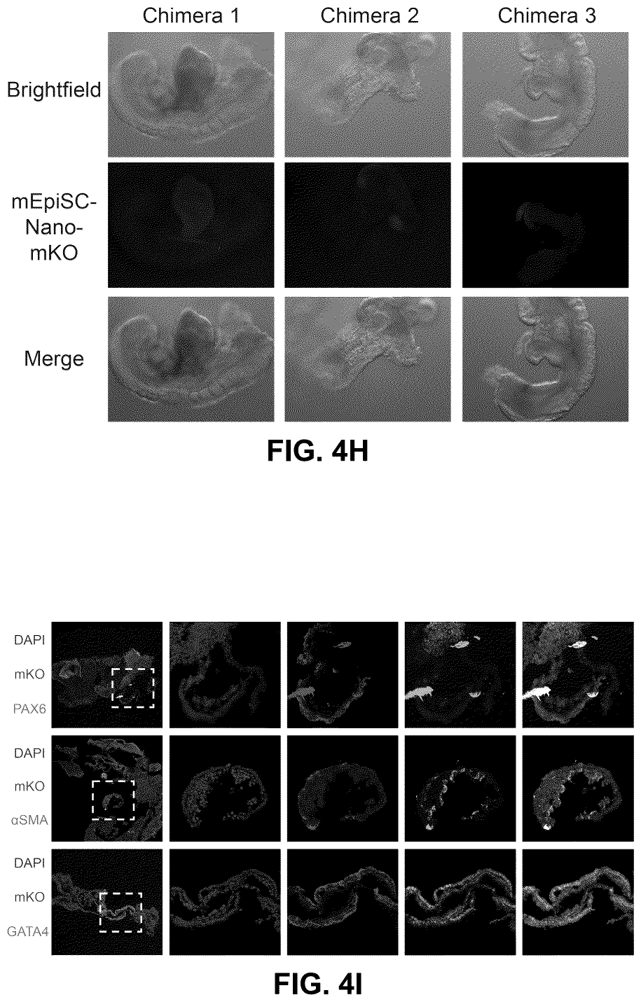

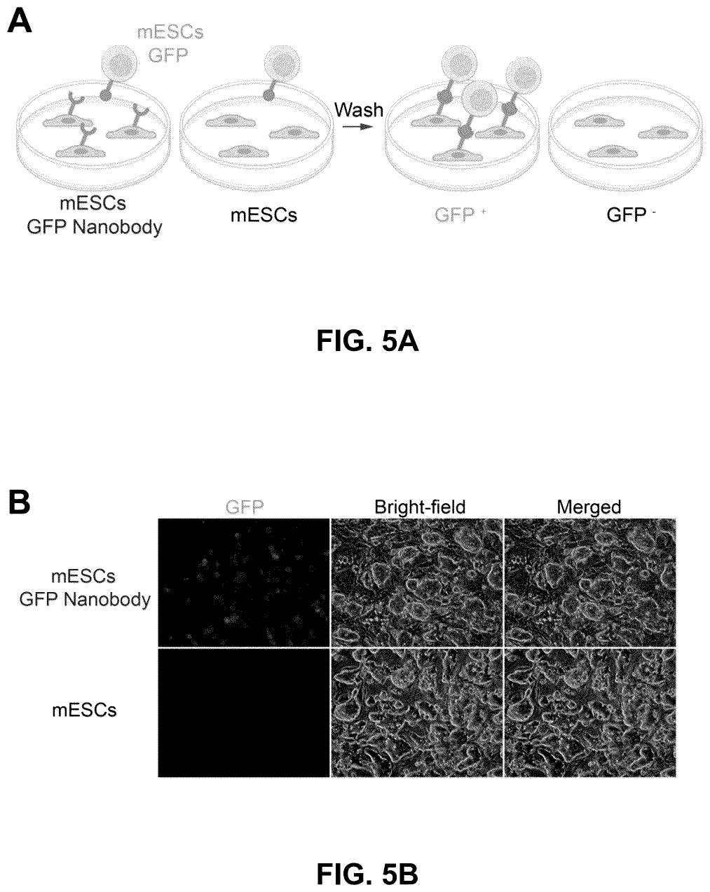

Building on the in vitro success of the membrane-localized nanobody adhesion system in enhancing adhesion between mouse and human PSCs, its impact in vivo was investigated to see if it would lead to increased human contribution to interspecies chimeras. Mice globally expressing GPI-anchored GFP were used (FIG. 4A), which had been previously established and characterized (Rhee et al., Genesis (2006), 44:202-218). Nano-GPI PSCs was employed for the in vivo experiments. First, it was assessed whether enforced cell adhesion had any adverse effects on embryo development, by generating intraspecies chimeras using donor Nano-GPI mESCs injected into GFP-GPI host blastocysts. It was found that the artificial cell adhesion did not disrupt development, as the chimeric embryos appeared grossly normal at mid-gestation and gave rise to live-born pups with visible coat color changes (FIGS. 2A-2B). Nano-GPI mESCs significantly contributed to E12.5 chimeric embryos and differentiated into cells expressing markers of all three germ layers (FIG. 4C). A summary of the results for the intraspecies and interspecies chimera experiments are shown in TABLES 1A-1B.

TABLE 1A
Summary of intraspecies and interspecies chimera experiments

| | Donor cell | Cell culture | Cells injected | Embryos injected |
|---|---|---|---|---|
| Donor cell line | species | media | per embryo | (E3.5) |
| hiPSC MyD88 KO + mKO (#4) | Human | 4CL | 10 | 77 |
| hiPSC MyD88 KO + mKO + Nano GPI (#4) | Human | 4CL | 10 | 144 |
| hiPSC + mKO + Nano GPI (#5) | Human | mTeSR1 | 10 | 51 |
| mESC B6-4 + mKO + Nano GPI (#1) | Mouse | NB2iL | 10 | 16 |
| mESC B6-4 + mKO + Nano GPI (#1) | Mouse | NB2IL | 10 | 63 |
| mESC B6-4 + mKO + Nano GPI (#3) | Mouse | NB2iL | 10 | 64 |
| mEpiSC A1 + mKO + Nano GPI (#3) | Mouse | NBFR | 10 | 25 |

TABLE 1B
Summary of intraspecies and interspecies chimera experiments (continuation of TABLE1A)

| | | mKO | Genomic | | % Positive |
|---|---|---|---|---|---|
| | | fluorescent | qPCR | % Positive | Embryos |
| | Embryos | postive | positive | Embryos | (Genomic |
| Donor cell line | recovered | embryos | embryos | (Fluorescence) | qPCR) |
| hiPSC MyD88 KO + mKO (#4) | 37 | 9 | 3 | 24.3% | 8.1% |
| hiPSC MyD88 KO + mKO + Nano GPI (#4) | 43 | 12 | 20 | 27.9% | 46.5% |
| hiPSC + mKO + Nano GPI (#5) | 22 | 10 | 9 | 45.5% | 40.9% |
| mESC B6-4 + mKO + Nano GPI (#1) | 5 | 4 | N/A | 80.0% | N/A |
| mESC B6-4 + mKO + Nano GPI (#1) | 29 | 18 | N/A | 62.1% | N/A |
| mESC B6-4 + mKO + Nano GPI (#3) | 12 | 7 | N/A | 58.3% | N/A |
| mEpiSC A1 + mKO + Nano GPI (#3) | 20 | 7 | N/A | 35.0% | N/A |

Interspecies chimeras were next generated using Nano-GPI hiPSCs as donor cells. These hiPSCs were cultured under two naïve-like conditions (LCDM18 and 4CL33 media) and genetically modified to knock out myeloid differentiation primary response 88 (MyD88), an innate immunity signaling adaptor protein whose loss reduces interspecies PSC competition. Similar to the intraspecies experiments, these Nano-GPI hiPSCs were injected into GFP-GPI mouse blastocysts, which were then transferred to surrogate mothers.

Next, interspecies chimeras were generated using control (not expressing nanobody) and Nano-GPI hiPSCs that lack MyD88 (to minimize cell competition) (Zheng et al., Nature (2021), 592:272-276). These hiPSCs were cultured in naïve 4CL medium (Mazid et al., Nature (2022), 605:315-324) to match the developmental stage of injected hiPSCs to the host blastocysts. These control and Nano-GPI hiPSCs were injected into GFP-GPI mouse blastocysts, which were then transferred to surrogate mothers and harvested at E8.5. Imaging of chimeric embryos using a fluorescent microscope revealed areas of red fluorescent signal from donor hiPSCs in some embryos (FIGS. 2D and 4D). To further quantify the human cell contribution in these embryos, mitochondrial genomic qPCR and immunohistochemistry (IHC) techniques were employed, with both targeting human-specific mitochondrial markers. The mitochondrial qPCR assay indicated that Nano-GPI hiPSCs contributed to E8.5 chimeric embryos at a significantly higher rate than control hiPSCs (FIGS. 2E-2G and TABLES 1A-1B). Notably, a higher percentage of embryos per embryo transfer derived from Nano-GPI hiPSCs surpassed the positive qPCR threshold (equivalent to 1 human cell in 10,000 mouse cells) compared to those derived from control hiPSCs (FIG. 2F). Additionally, chimeric embryos derived from Nano-GPI hiPSCs contained a higher average human DNA content per embryo than those from control hiPSCs (FIG. 2G). These findings were confirmed by repeating the qPCR assay with some of the embryos using a different primer (FIGS. 4E-4G). Furthermore, IHC staining of frozen sections from the chimeric embryos revealed that Nano-GPI hiPSCs could differentiate within the embryos and contribute to tissues derived from all three germ layers (FIG. 2H).

In addition to overcoming the interspecies adhesion barrier, it was investigated whether membrane-localized nanobody adhesion could address heterochrony, the mismatch in developmental stages between donor PSCs and the host embryo. Primed PSCs usually cannot contribute to chimera formation when injected into blastocysts without blocking cell apoptosis (Wang et al., Cell Res (2018), 28:126-129; Masaki et al., Cell Stem Cell (2016), 19:587-592) or cell competition (Zheng et al., Nature (2021), 592:272-276). Interestingly, a previous study found that overexpressing the adhesion protein E-cadherin enables mEpiSCs to contribute to blastocyst chimeras (Ohtsuka et al., PLOS One (2012), 7). This suggests that enhancing cell adhesion using our method might help overcome heterochrony as well. To test this, intraspecies chimera formation was first examined by injecting Nano-GPI mEpiSCs into GFP-GPI mouse blastocysts. Visible areas of red fluorescence were observed in 7 out of 20 recovered E8.5 embryos (FIG. 4H and TABLES 1A-1B). IHC on sections from these chimeric embryos showed that Nano-GPI mEpiSCs differentiated into cells expressing markers of all three embryonic germ layers (FIG. 4I). Nano-GPI hiPSCs cultured in a primed (mTeSR1) condition were injected into GFP-GPI mouse blastocysts. This also resulted in successful interspecies chimera formation at E8.5 (TABLES 1A-1B). These findings demonstrated that membrane-localized nanobody adhesion can help overcome heterochrony and enable chimeric contribution from primed PSCs in both intra- and inter-species contexts.

### Discussion

This study performed an in-depth analysis of the interspecies cell adhesion barrier by co-culturing PSCs from different species. A synthetic biology approach was developed, utilizing interactions between membrane-localized nanobodies and antigens to overcome this barrier. This approach significantly improved cell adhesion between mouse and human PSCs in vitro, as demonstrated by multiple adhesion assays. Importantly, the in vivo studies show that this synthetic cell adhesion system can also improve the chimeric contribution of human cells into mouse embryos. By attaching the nanobody and antigen to the cell's outer membrane using a GPI anchor, cell adhesion was enhanced in an orthogonal manner, which does not interfere with natural cell junctions or cellular signaling. This synthetic cell adhesion strategy enables interspecies organogenesis across a broader spectrum of species.

### Materials and Methods

All experiments followed the 2021 Guidelines for Stem Cell Research and Clinical Translation released by the International Society for Stem Cell Research (ISSCR). All human-mouse ex vivo and in vivo interspecies chimeric experimental studies were reviewed and approved by the University of Texas Southwestern Stem Cell Oversight Committee (SCRO).

Nanobody and GFP cell-surface constructs

The pOPINE vhhGFP4 anti-GFP nanobody plasmid (Addgene plasmid #49172; RRID: Addgene_49172) was gifted. The Nano-GPI construct was generated by fusing a membrane-targeting signal sequence to the N-terminus of the nanobody and a GPI anchor signal to the C-terminus via Gibson assembly. Membrane-targeted GFP constructs were generated using the same strategy. The plasmid containing the vhhGFP4 anti-GFP nanobody fused to the CD8 transmembrane sequence was gifted. Nano-GPI and Nano-CD8 sequences were cloned into a vector with expression driven by the CAG promoter and containing 5′ and 3′ inverted terminal repeat (ITR) sequences to allow for transposition and genomic integration when co-transfected with a Piggybac Transposase plasmid.

### Transgenic Mouse Lines

Mice with global expression of the GFP-GPI construct were purchased as a cryorecovery strain (#011106) from the Jackson Laboratory, and the colony was expanded by breeding heterozygotes to generate a homozygous transgenic strain.

### Cell Culture

Naïve mESCs derived from the C57BL/6 strain were maintained in the ground state of pluripotency by culture in 2i/LIF medium (Ying et al., Nature (2008), 453:519-523). Primed H9 hESCs, HFF-iPSCs, and mEpiSCs were maintained in NBFR medium (Wu et al., Nature (2015), 521:316-321), with the addition of 10 uM ROCKi (Tocris) during passaging and for 24 hours after each passage for the human cells. Primed mEPISCs and hPSCs cultured in NBFR medium were used for in vitro cell adhesion assays unless otherwise specified. For use in interspecies chimera experiments, hiPSCs were converted to the naïve state by culturing them in 4CL medium (first described by Mazid et al., Nature (2022), 605:315-324) containing the histone deacetylase (HDAC) inhibitor Trichostatin A and the H3K27 methyltransferase inhibitor DZNep for a minimum of three passages before injection into mouse blastocysts. During passaging, cultured cells were treated with TrypLE for 3 minutes at 37 C to detach them from the plate, then counted using a hemocytometer. Cells were transfected using the Lonza 4D Nucleofector in P3 primary cell solution in order to generate nanobody and GFP-expressing cell lines.

### Immunofluorescence Staining of Cell Junctions

Unlabeled mEpiSCs and mKO-labeled hiPSCs were plated at a 1:1 ratio for a total density of 100,000 cells per well on top of glass coverslips in 12 well gelatin-coated dishes with a feeder layer of mouse embryonic fibroblasts (MEFs). Cells were cultured in NBFR+10 uM ROCKi for 24 hours, followed by another 24 hours in NBFR without ROCK inhibitor. After 48 hours of co-culture, cells were fixed in 4% paraformaldehyde (PFA)/PBS for 30 minutes at room temperature (RT), then permeabilized in 0.1% Triton X-100 for 5 minutes at RT. Blocking was carried out in 5% donkey serum diluted in PBST (PBS+0.1% Tween-20) for 1 hour at RT. ZO1 and E-cadherin primary antibodies were diluted 1:200 each in blocking buffer (as described above) and incubated overnight at 4° C. After washing with PBST, samples were incubated with Alexa Fluor 488 conjugated secondary antibodies diluted in blocking buffer for 1 hr at RT, followed by nuclear staining with 1:3000 DAPI diluted in PBS at RT for 20 minutes. Coverslips were mounted onto slides in ProLong Gold Antifade Mountant, and slides were imaged on a LSM700 confocal microscope. Mouse and human cells were distinguished from one another by the presence of mKO red fluorescent protein in the human PSCs.

### Immunofluorescence Staining of Nanobody and Pluripotency Marker Expression

WT, Nano-GPI, and Nano-CD8 mESCs were plated at a density of 100,000 cells per well on glass coverslips in 12 well gelatin-coated plates with a MEF feeder layer. Cells were cultured in NB2iL medium for 48 hours, then fixed in 4% PFA for 30 minutes at RT and permeabilized in 0.1% Triton X-100 for 5 minutes (nanobody) or 15 minutes (OCT4/SOX2). Blocking was carried out in 5% goat serum (nanobody staining) or donkey serum (OCT4/SOX2 staining) diluted in PBST (PBS+0.1% Tween-20) for 1 hour at RT. OCT4 and SOX2 primary antibodies were diluted 1:200 each in blocking buffer and incubated overnight at 4° C. For nanobody staining, the primary antibody was omitted, and cells were incubated directly with Alexa Fluor 647 conjugated goat anti-alpaca secondary antibody diluted 1:100 in blocking buffer at 4° C. overnight. After washing with PBST, OCT4/SOX2 staining samples were incubated with Alexa Fluor 488 conjugated secondary antibodies diluted in blocking buffer for 1 hr at RT. All samples were washed with PBST, then counterstained with 1:3000 DAPI diluted in PBS at RT for 20 minutes. Coverslips were mounted onto slides in ProLong Gold Antifade Mountant, and slides were imaged on a LSM700 confocal microscope.

### Correlative Light-Electron Microscopy

GFP-GPI mEpiSCs and mKO-labeled WT or Nano-GPI hiPSCs were plated in a 1:1 ratio at low density and co-cultured in a 35 mm gridded MatTek glass bottom dish. After 48 hours, cells were fixed in 4% PFA/7.5% sucrose, then imaged via fluorescent microscopy to establish locations of mouse and human cells within the grid. Mouse and human cells were distinguished by red fluorescent labeling of human PSCs and green fluorescent labeling of mouse PSCs. Cells were then post-fixed in 1% glutaraldehyde and washed in 50 mM glycine, then submitted to the University of Texas Southwestern Electron Microscopy core facility for processing and contrast enhancement. Cells were imaged via transmission electron microscopy (TEM) at magnifications between 1000× and 10,000×. Locations of human and mouse cells within mixed colonies were determined by comparing cell shape and location on electron microscopy images with the fluorescence microscopy images previously taken at lower magnification. TEM images were labeled, colorized, and analyzed in FIJI.

### GFP Binding Assay

The ability of Nano-GPI and Nano-CD8 PSCs to bind GFP was measured by adding 5 μg of purified GFP protein to the cell culture media. After 1 hour of culture in GFP-containing medium, cells were imaged using a fluorescence microscope. All nanobody-expressing cell lines demonstrated the ability to capture free-floating GFP and display it on their cell membranes.

### Flow Cytometry Doublet Assay

Differentially labeled primed PSCs (mEpiSCs expressing GFP-GPI and hPSCs/mEpiSCs labeled with monomeric Kusabira Orange (mKO)) were combined at a ratio of 1:1 for a total of 400,000 cells per well. This cell mixture was plated in a 24-well low-adhesion dish (Corning) and agitated at 80 rpm on an orbital shaker at 37° C. for 1 hour. The cells were filtered through a 70 μm cell strainer to break up large clusters and analyzed using a BD LSRII flow cytometer. An expanded live cell gate was used to capture doublets, and only the resulting doublets were analyzed for GFP and mKO fluorescence using FlowJo software. In human+pig doublet assays, cells were cultured in a chemically defined medium first described by Choi et al (Choi et al., Stem Cell Reports (2019), 13:221-234). In human+monkey doublet assays, cells were cultured in mTESR1. For human+mouse and human+rat doublet assays, cells were cultured in NBFR medium in the primed state of pluripotency unless otherwise specified. In addition to primed cells, the doublet assay was also performed on cells cultured in the naïve 4CL condition and after one week of random differentiation in serum-containing medium (FIG. 1F).

### 3D Co-Aggregation Assay

Red and green fluorescently labeled primed PSCs were combined at a ratio of 1:1 for a total of 15,000 cells per sample. The cell mixtures were plated in an Aggrewell 800 plate, resulting in an average aggregate size of 50 total cells per microwell. Aggregates were imaged after 24 hours of culture. For human+mouse co-aggregation studies, aggregates were cultured in NBFR medium. In human+pig co-aggregation studies, aggregates were cultured in a chemically defined medium first described by Choi et al (Choi et al., Stem Cell Reports (2019), 13:221-234). In human+monkey co-aggregation studies, aggregates were cultured in mTESR1. After 24 hours of culture, aggregates were imaged, and each aggregate was scored according to whether it was predominantly intermixed or predominantly segregated (showing a clear line of demarcation between two differently colored cell clusters, with fewer than three cells crossing this midline). Percentages of intermixed aggregates were compared between samples using Student's t-test (between pairs of species) or one-way ANOVA (between more than two samples, such as in FIG. 1P).

### Wash Assay

Ibidi 2-well cell culture inserts were placed in each well of an Ibidi 8-well chamber plate, then coated with a solution of 0.5% Matrigel for one hour at 37° C. 100,000 red fluorescently labeled mEpiSCs, hESCs, or hiPSCs were seeded per side of each culture insert in NBFR medium+ROCKi and allowed to attach to the plate overnight. On the following day, the culture insert was removed and 100,000 GFP-GPI mEpiSCs were seeded per well. After 30 minutes, the cell suspension was removed and each well was washed with PBS three times to remove any unattached cells, followed by fixation in 4% PFA for 20 minutes. Samples were imaged using a Zeiss LSM880 confocal microscope and the number of remaining attached green cells per culture insert was quantified in FIJI.

### Lumen Formation Assay

Red and green fluorescently labeled PSCs were combined at a ratio of 1:1 for a total of 3000 cells per sample. This dilute cell mixture was plated in an Aggrewell 800 plate, resulting in an average aggregate size of 10 total cells per microwell. After 4 hours, the medium (NBFR+ROCKi) was carefully replaced with fresh medium containing 10% Matrigel. Aggregates were cultured for 48 hours in this ECM overlay, which was sufficient for cells to polarize and begin to form lumens. After 48 hours, aggregates were harvested, fixed for 30 minutes in 4% PFA, and permeabilized for 15 minutes with 0.1% Triton X-100. Aggregates were stained with Alexa Fluor 647-conjugated phalloidin dye recognizing the actin protein, and counterstained with DAPI. Aggregates were imaged on the LSM880 confocal microscope. Most aggregates were composed of a mixture of mouse and human cells, and only these chimeric aggregates were considered. Chimeric aggregates were scored according to both the number of human cells they contained and the percentage of these human cells in direct contact with the phalloidin-stained lumen. Percentages of polarized human cells in contact with the lumen were compared between WT-hiPSC and Nano-GPI hiPSC aggregates using Student's t-test.

### Random Differentiation

Mouse and human primed PSCs were plated at a density of 100,000 cells per well onto a 6-well cell culture plate coated with Matrigel in N2B27 basal medium containing 10% FBS. Cells were allowed to randomly differentiate in this serum-containing medium for one week with media changes every other day, then dissociated and used for doublet flow cytometry assays.

### Teratoma Formation

Nano-GPI and Nano-CD8 mESCs at a concentration of 10,000,000 cells/mL suspended in 50% Matrigel and 50% NB2iL culture medium were injected subcutaneously into the flanks of NOD/SCID immunodeficient mice. Three NOD/SCID mice were used, each with one flank receiving Nano-GPI mESCs and the other receiving Nano-CD8 mESCs. 1 million cells were injected per tumor, and this strategy generated three tumors per cell type. Mice were monitored for tumor growth and sacrificed four weeks after cell injection. Tumors were dissected and fixed in 4% PFA for 48 hours, then submitted to the UT Southwestern Histology Core facility for paraffin embedding, sectioning, and H&E staining.

### Intraspecies Chimera Studies

Female GFP-GPI mice were superovulated via injection of PMSG and hCG, then mated with male GFP-GPI mice. Blastocysts were harvested at E3.5 and microinjected with either 10 naïve Nano-GPI mESCs (derived from the C57BL/6 strain) or 10 primed Nano-GPI mEpiSCs (for heterochrony studies) each, then transferred to pseudopregnant CD-1 surrogate females and allowed to develop until E8.5 (mEpiSCs for heterochrony), E12.5 (naïve mESCs), or until the surrogates gave birth naturally at E19.5 (naïve mESCs). E8.5 and E12.5 embryos were dissected and imaged using a fluorescence stereomicroscope, then fixed in 4% PFA/0.1% PVA/PBS overnight at 4° C. Embryos were permeabilized in 30% sucrose for 24 hours, then embedded into tissue blocks in OCT compound and frozen until sectioning. 12 m tissue sections were cut using a Leica cryostat and mounted onto gelatin-coated slides, which were then analyzed via immunohistochemistry. Live-born pups were imaged for coat color change indicating chimerism at one week of age.

### Interspecies Chimera Studies

Female GFP-GPI mice were superovulated via injection of PMSG and hCG, then mated with male GFP-GPI mice. Blastocysts were harvested at E3.5 and microinjected with either 10 control or Nano-GPI hiPSCs each, then transferred to pseudopregnant CD-1 surrogate mothers and allowed to develop until E8.5. E8.5 embryos were dissected and imaged using a fluorescence stereomicroscope, then fixed in 4% PFA/0.1% PVA/PBS overnight at 4° C. Embryos were permeabilized in 30% sucrose for 24 hours, then embedded into tissue blocks in OCT compound and frozen until sectioning. 12 μm sections were cut using a Leica cryostat, and alternating sections were mounted onto slides or reserved for DNA extraction. Genomic DNA was extracted from tissue sections using the Qiagen DNeasy kit and used for mitochondrial genomic qPCR. Slides containing tissue sections were analyzed via immunohistochemistry.

Mitochondrial Genomic Quantitative PCR (qPCR)

Genomic DNA was isolated from interspecies teratomas and interspecies chimeric embryos, as well as known ratios of mixed mouse and human cultured PSCs. DNA was isolated using the Qiagen DNasy Blood and Tissue Kit, then diluted to a concentration of 5 ng/μL for a final amount of 10 ng of DNA per reaction. qPCR reactions were set up using the SYBR Green 2× Mastermix using primers targeting human-specific sequences in the mitochondrial genome (see TABLE 2).

TABLE 2
Primer sequences used for mitochondrial genomic qPCR

| Primer Name | Target | Sequence | SEQ ID NO |
|---|---|---|---|
| Human Mito 1 F | DNA encoding Human | AATATTAAACACAAACTACCACCTA | 114 |
| | Mitochondrial ATP Synthase | CCT | |
| | Subunit 8 | | |
| Human Mito 1 R | DNA encoding Human | TGGTTCTCAGGGTTTGTTATAA | 115 |
| | Mitochondrial ATP Synthase | | |
| | Subunit 8 | | |
| Human Mito 2 F | Human Mitochondrial | CGGGAGCTCTCCATGCATTT | 116 |
| | Genomic DNA | | |
| Human Mito 2 R | Human Mitochondrial | GACAGATACTGCGACATAGGGT | 117 |
| | Genomic DNA | | |
| Common Mito 1 F | DNA encoding | GCTAAGACCCAAACTGGGATT | 118 |
| | Human/Mouse Mitochondrial | | |
| | 12s rRNA | | |
| Common Mito 1 R | DNA encoding | GGTTTGCTGAAGATGGCGGTA | 119 |
| | Human/Mouse Mitochondrial | | |
| | 12s rRNA | | |

qPCR was performed on the Bio-Rad CFX384 thermocycler for a total of 40 cycles, then analyzed using the CFX Manager software and Microsoft Excel, as well as GraphPad Prism for statistics and visualization. Three technical replicates were used per sample, and CT values were normalized to those of a common sequence that is identical between human and mouse mitochondrial genomes. 2-delta-CT values were calculated and compared to the 2DCTs of known mouse-human cell mixtures. These known mixtures served as a standard curve of human DNA percentage, and a linear regression equation was used to calculate the percentage of human DNA in chimeric embryos.

### Immunohistochemistry (Frozen Tissue)

Frozen sections from chimeric embryos were mounted onto gelatin-coated slides and stained via the following IHC protocol: Tissue sections were washed in PBS, then permeabilized in 0.3% Triton X-100/PBS. Antigen retrieval was performed by incubation in 10 mM citrate buffer (pH 6.0) at 90° C. for 10 minutes. Samples were blocked in blocking buffer containing 5% donkey or goat serum (depending on the host species of secondary antibodies in each experiment) and 0.3% Triton X-100 in PBS for 1 hour at room temperature, then incubated overnight at 4° C. with primary antibodies diluted in blocking buffer. All primary antibodies were diluted 1:200 aside from the human mitochondrial antibody, which was diluted 1:800. Fluorescently conjugated donkey or goat secondary antibodies diluted 1:200 in blocking buffer were applied for 1 hour at RT, then samples were counterstained with 1:10,000 DAPI for 15 minutes. Autofluorescence was quenched using TrueBlack Lipofuscin (Cell Signaling Technologies), and coverslips were mounted onto slides in ProLong Gold Antifade Mountant (Invitrogen). Slides were imaged on the LSM700 confocal microscope.

TABLE 3
List of materials and resources

| Reagent or Resource | Source | Identifier |
|---|---|---|
| Antibodies | | |
| GFP antibody, Goat polyclonal | Rockland | Catalog #: 600-101-215, |
| | | RRID: AB_218182 |
| GFP antibody, Rabbit polyclonal | Rockland | Catalog #: 600-401-215, |
| | | RRID: AB_828167 |
| Anti-monomeric Kusabira- | MBL International | Catalog #: M168-3M |
| Orange 2 mAb (Monoclonal | Corporation | RRID: AB_10597268 |
| Antibody) | | |
| Anti-Mitochondria antibody | Abcam | Catalog #: ab92824, |
| [113-1] | | RRID: AB_10562769 |
| PAX6 Polyclonal antibody | Invitrogen | Catalog # 42-6600, |
| | | RRID: AB_2533534 |
| Alpha-Smooth Muscle Actin | Invitrogen | Catalog # MA1-06110, |
| Monoclonal Antibody (1A4) | | RRID: AB_557419 |
| Recombinant Anti-GATA4 | Abcam | Catalog #: ab134057 |
| antibody [EPR23691-12] | | RRID: AB_2725747 |
| Foxa2/hnf3β (D56D6) XP ® | Cell Signaling Technology | Catalog # 8186S |
| Rabbit mAb | | RRID: AB_10891055 |
| Oct3/4 Antibody (C-10) | Santa Cruz Biotechnology | Catalog #: sc-5279, |
| | | RRID: AB_628051 |
| Sox-2 (E-4) antibody | Santa Cruz Biotechnology | Catalog #: sc-365823, |
| | | RRID: AB_10842165 |
| ZO-1 Monoclonal antibody | Invitrogen | Catalog # 33-9100, |
| | | RRID: AB_87181 |
| E-Cadherin (24E10) Rabbit mAb | Cell Signaling Technology | Catalog #: 3195, |
| | | RRID: AB_2291471 |
| Donkey anti-goat IgG (H + L) | Invitrogen | Catalog # A-11055, |
| Antibody, Alexa Fluor ™ 488 | | RRID: AB_2534102 |
| Donkey anti-rabbit IgG (H + L) | Invitrogen | Catalog # A-21206, |
| Antibody, Alexa Fluor ™ 488 | | RRID: AB_2535792 |
| Donkey anti-mouse IgG (H + L) | Invitrogen | Catalog # A-31570, |
| Antibody, Alexa Fluor ™ 555 | | RRID: AB_2536180 |
| Donkey anti-rabbit IgG (H + L) | Invitrogen | Catalog # A-31572, |
| Antibody, Alexa Fluor ™ 555 | | RRID: AB_162543 |
| Donkey anti-mouse IgG (H + L) | Invitrogen | Catalog # A-31571, |
| Antibody, Alexa Fluor ™ 647 | | RRID: AB_162542 |
| Donkey anti-rabbit IgG (H + L) | Invitrogen | Catalog # A-31573, |
| Antibody, Alexa Fluor ™ 647 | | RRID: AB_2536183 |
| Goat anti-Mouse IgG (H + L) | Invitrogen | Catalog # 31430, |
| Secondary Antibody, HRP | | RRID: AB_228307 |
| Goat Anti-Mouse IgG H&L | Abcam | Catalog #: ab39619, |
| (10 nm gold) | | RRID: AB_954440 |
| Goat anti-Mouse IgG1 Cross- | Invitrogen | Catalog #: .A21127, |
| Adsorbed Secondary Antibody, | | RRID: AB_2535769 |
| Alexa Fluor ™ 555 | | |
| Goat anti-Mouse IgG2a Cross- | Invitrogen | Catalog #: .A21241, |
| Adsorbed Secondary Antibody, | | RRID: AB_2535810 |
| Alexa Fluor ™ 647 | | |
| Goat anti-Rabbit IgG (H + L) | Invitrogen | Catalog # A-11008, |
| Cross-Adsorbed Secondary | | RRID: AB_143165 |
| Antibody, Alexa Fluor ™ 488 | | |
| Alexa Fluor ® 647 AffiniPure ™ | Jackson ImmunoResearch | Catalog # 128-605-160 |
| Goat Anti-Alpaca IgG (H + L) | | RRID: AB_2783782 |
| Chemicals, Peptides, | | |
| Recombinant Proteins | | |
| Alexa Fluor ® 647 Phalloidin | Invitrogen | Catalog #: A22287 |
| Recombinant Human FGF-basic | Peprotech | Catalog # 100-18B |
| Endo-IWR 1 | Tocris | Catalog # 3532 |
| Recombinant human LIF | Peprotech | Catalog # 300-05 |
| CHIR99021 | Selleckchem | Catalog # S1263 |
| PD0325901 | Selleckchem | Catalog # S1036 |
| Geltrex ™ LDEV-Free Reduced | Gibco | Catalog # A1413201 |
| Growth Factor Basement | | |
| Membrane Matrix | | |
| Corning ® Matrigel ® hESC- | Corning | Catalog # 354277 |
| Qualified Matrix, LDEV-free | | |
| N2 supplement (100X) | Gibco | Catalog # 17502-048 |
| B27 supplement (50X) | Gibco | Catalog # 17504-044 |
| Recombinant Human/Murine/Rat | Peprotech | Catalog # 120-14E |
| Activin A | | |
| mTeSR ™ Plus | STEMCELL Technologies | Catalog # 100-1130 |
| WH-4-023 | Tocris | Catalog #. 5413 |
| DMEM/F12 | Gibco | Catalog # 11320-033 |
| Advanced DMEM/F12 | Gibco | Catalog #: 12634010 |
| GlutaMAX (100X) | Gibco | Catalog # 35050-061 |
| MEM Non-Essential Amino | Gibco | Catalog # 11140-050 |
| Acids (100X) | | |
| Sodium Pyruvate | Sigma Aldrich | Catalog # S8636-100ML |
| 2-Mercaptoethanol (1000X) | Gibco | Catalog # 21985-023 |
| Fetal Bovine Serum | Sigma | Catalog # 1270548 |
| XAV-939 | Tocris | Catalog # 3748 |
| L-Ascorbic acid 2-phosphate | Sigma-Aldrich | Catalog # A8960 |
| Trichostatin A | Sigma-Aldrich | Catalog # V900931 |
| DZNep | Selleck Chemicals | Catalog # S7120 |
| OptiMEM Reduced Serum | Thermo Fisher Scientific | Catalog # 31985088 |
| Medium | | |
| Trichostatin A | Neta Scientific | Catalog # AST-43193 |
| 3-deazaneplanocin A (DZNeP) | Selleck Chemicals | Catalog # S7120 |
| Green Fluorescent Protein | Millipore | Catalog #14-392 |
| DAPI | Thermo Fisher Scientific | Catalog # D3571 |
| DNase I | Thermo Fisher Scientific | Catalog # 18047019 |
| 2x SYBR Green Mastermix | APExBIO | Catalog # K1070 |
| Y-27632 | APExBIO | Catalog # A3008 |
| KnockOut Serum Replacement | Gibco | Catalog # 10828028 |
| Gelatin | Sigma | Catalog # G1850-500g |
| Donkey serum | Sigma | Catalog # D9663 |
| Goat serum | Sigma | Catalog # G9023 |
| Bovine serum albumin | Sigma | Catalog t# A6003 |
| Paraformaldehyde 16% Aqueous | Electron Microscopy | Catalog # 15710 |
| Solution EM Grade | Sciences | |
| Triton X-100 | Sigma | Catalog # T8787 |
| Tween 20 | Sigma | Catalog # P9416 |
| RIPA buffer | Sigma | Catalog # R0278 |
| Protease and phosphatase | Sigma | Catalog # PPC1010 |
| inhibitor | | |
| TrueBlack ® Lipofuscin | Biotium | Catalog # 23007 |
| Autofluorescence Quencher | | |
| ProLong ™ Gold Antifade | Invitrogen | Catalog # P36934 |
| Mountant | | |
| Anti-Adherence Rinsing Solution | STEMCELL Technologies | Catalog #. 07,010 |
| TrypLE ™ Express | Gibco | Catalog # 12605036 |
| NuPAGE ™ LDS Sample Buffer | Invitrogen | Catalog # NP0008 |
| (4X) | | |
| Card HyperOva PMSG | Cosmo Bio | Catalog # KyD-010-EX-X5 |
| hCG Protein | ProSpec | Catalog # HOR-250 |
| Software and algorithms | | |
| Illustrator | Adobe | Adobe Illustrator 2022 |
| Excel | Microsoft | Microsoft Excel V16.0 |
| FIJI | Schindelin et al., 2012 | N/A |
| GraphPad Prism | Dotmatics | GraphPad Prism 10.1.2 |
| FlowJo | BD (Becton, Dickinson & | See FlowJo website |
| | Company) | |
| CFX Manager | Bio-Rad | N/A |
| BioRender | BioRender | See BioRender website |
| EndNote | Microsoft | EndNote 20 |
| Other | | |
| AggreWell 800 | STEMCELL Technologies | Catalog # 34815 |
| BD LSR II Flow cytometer | BD Biosciences | N/A |
| Zeiss LSM700 confocal | Zeiss | N/A |
| microscope | | |
| Zeiss LSM880 confocal | Zeiss | N/A |
| microscope | | |
| ECHO Revolve microscope | Echo | N/A |
| Stereo Microscope SMZ800N | Nikon | Catalog # SMZ800N |
| CFX384 Touch Real-Time PCR | Bio-Rad | Catalog # 1855484 |
| Detection System | | |
| μ-Slide 8-well chamber slide | Ibidi | Catalog # 80821 |
| Costar ® 24-well Clear Flat | Corning | Catalog # 3473 |
| Bottom Ultra-Low Attachment | | |
| Multiple Well Plates | | |
| 4D Nucleofector X Unit | Lonza | Catalog # AAF-1003X |
| P3 Primary Cell 4D- | Lonza | Catalog # V4XP-3024 |
| Nucleofector ® X Kit L | | |
| Dneasy Blood & Tissue Kit | Qiagen | Catalog # 69504 |
| PureLink ™ HiPure Plasmid | Invitrogen | Catalog #K210002 |
| Miniprep Kit | | |
| 25 Culture-Inserts 2 Well | Ibidi | Catalog # 80209 |

### Example 2: Improvements to Cellular Adhesion

In an effort to improve cell adhesion, optimization of the cell adhesion proteins was performed. Notably, the Nano-GPI constructs prepared in Example 1 contained a membrane-targeting signal sequence and GPI anchor signal, which localize the cell-adhesion nanobodies at the cell membrane. It was recognized that during cell adhesion, this peripheral positioning has the potential to instigate instability of adhesion and potentially tear the cells apart. To enhance the stability of cell adhesion, it was hypothesized that implementing a means for integrally anchoring the nanobodies or EGFP could solve this problem. Accordingly, the nanobodies and/or EGFP were fused with the cytoskeletal protein intercellular adhesion molecule 1 (ICAM1). With this solution, integration with the cytoskeleton improves the stability of cellular adhesion. To achieve this, plasmids having ICAM1 cell membrane anchor proteins were designed, according to TABLE 4 below.

TABLE 4
Adhesion constructs comprising ICAM1
cell membrane anchor proteins

| Construct | |
|---|---|
| Description | Design |
| nanobody-ICAM1 | PiggyBac (PB)_TRE3G |
| | Promoter_GFP_Nano_hICAM1_HA_EF1a |
| | Promoter_Tet-on 3G_PuroR_AmpR |
| EGFP-ICAM1 | PB_CAG Promoter_EGFP_hICAM1_AmpR |

To verify whether the nanobody fused to the cytoskeleton can efficiently bind EGFP, wash assay experiments were performed according to protocols described in Example 1. Plasmids expressing GFP nanobody or GFP were incorporated into the mESCs genome using the PiggyBac transposon system, resulting in the mESCs GFP nanobody and mESCs GFP cell lines, respectively (FIG. 5A). 100,000 mESCs expressing GFP nanobody were seeded on each side of the culture insert in NBFR medium and allowed to attach overnight. The next day, the culture insert was removed, and mESCs expressing GFP were seeded in each well. After 30 minutes, the cell suspension was aspirated, and each well was washed three times with PBS to remove any unattached cells. The samples were imaged using a Zeiss LSM880 confocal microscope. The results show that compared to mESCs, which do not express the GFP nanobody, mESCs expressing GFP nanobody can efficiently bind to mESCs expressing GFP (FIG. 5B). These results demonstrate that cell adhesion proteins bound to the cytoskeleton can efficiently facilitate cell binding.

Additionally, to ensure that cells specifically adhere to embryonic tissues rather than extra-embryonic tissues, plasmids were designed having EGFP expression driven by the Primed Oct4 promoter (see TABLE 5). This approached ensures that only epiblast cells, which form the embryo, express EGFP, allowing cells expressing nanobody to adhere exclusively to these tissues.

TABLE 5
Epiblast-directed construct

| Construct type | Design |
|---|---|
| Oct4-EGFP-ICAM1 | PB_Primed Oct4 |
| | Promoter_EGFP_hICAM1_AmpR |

To test the efficiency of these adhesion proteins in vivo, the EGFP-ICAM1 and Oct4-EGFP-ICAM1 plasmids, of TABLES 4 and 5, respectively, were injected along with PBase mRNA into mouse zygotes, generating mice expressing the plasmids

To test which binding strength is more favorable for cell-cell adhesion during embryonic development, nanobodies with varying strengths of GFP binding were incorporated into construct designs, as shown in TABLE 6.

TABLE 6
Epiblast-directed constructs comprising LAG nanobodies

| Construct type | Design |
|---|---|
| LAG5-ICAM1 | PB_TRE3G Promoter_Nano LAG5 (Kd = 14,200 nM)_hICAM1_HA_EF1a |
| | Promoter_Tet-on 3G_PuroR_AmpR |
| LAG6-ICAM1 | PB_TRE3G Promoter_Nano LAG6 (Kd = 310 nM)_hICAM1_HA_EF1a |
| | Promoter_Tet-on 3G_PuroR_AmpR |
| LAG8-ICAM1 | PB_TRE3G Promoter_Nano LAG8 (Kd = 20,000 nM)_hICAM1_HA_EF1a |
| | Promoter_Tet-on 3G_PuroR_AmpR |
| LAG10-ICAM1 | PB_TRE3G Promoter_Nano LAG10(Kd = 97 nM)_hICAM1_HA_EF1a |
| | Promoter_Tet-on 3G_PuroR_AmpR |
| LAG11-ICAM1 | PB_TRE3G Promoter_Nano LAG11(Kd = 22,900 nM)_hICAM1_HA_EF1a |
| | Promoter_Tet-on 3G_PuroR_AmpR |
| LAG17-ICAM1 | PB_TRE3G Promoter_Nano LAG17 (Kd = 50 nM)_hICAM1_HA_EF1a |
| | Promoter_Tet-on 3G_PuroR_AmpR |
| LAG18-ICAM1 | PB_TRE3G Promoter_Nano LAG18 (Kd = 3,800 nM)_hICAM1_HA_EF1a |
| | Promoter_Tet-on 3G_PuroR_AmpR |
| LAG27-ICAM1 | PB_TRE3G Promoter_Nano LAG27(Kd = 9.5 nM)_hICAM1_HA_EF1a |
| | Promoter_Tet-on 3G_PuroR_AmpR |
| LAG29-ICAM1 | PB_TRE3G Promoter_Nano LAG29 (Kd = 110 nM)_hICAM1_HA_EF1a |
| | Promoter_Tet-on 3G_PuroR_AmpR |
| LAG30-ICAM1 | PB_TRE3G Promoter_Nano LAG30 (Kd = 0.5 nM)_hICAM1_HA_EF1a |
| | Promoter_Tet-on 3G_PuroR_AmpR |
| LAG42-ICAM1 | PB_TRE3G Promoter_Nano LAG42 (Kd = 600 nM)_hICAM1_HA_EF1a |
| | Promoter_Tet-on 3G_PuroR_AmpR |

To verify whether the adhesion strategy could enhance the contribution of hPSCs to mouse embryos, nanobody plasmids with different binding affinities (i.e., the LAG5-ICAM1, LAG11-ICAM1, LAG18-ICAM1, and LAG42-ICAM1 constructs of TABLE 6) were incorporated into the hPSCs genome using the PiggyBac transposon system, generating the hPSCs.

1. A population of cells, comprising one or more cell of a first mammalian species and one or more cell of a second mammalian species, wherein the one or more cell of the first mammalian species comprises an exogenous nucleic acid encoding
(a) an antigen-binding protein that specifically binds to an antigen; and
(b) a cell membrane signaling peptide and/or a cell membrane anchor protein,
wherein the antigen-binding protein is expressed at the cell surface of the one or more cell of the first mammalian species and specifically binds to an antigen at the cell surface of the one or more cell of the second mammalian species, and
wherein the antigen is endogenous or exogenous to the one or more cell of a second mammalian species,
optionally wherein the population of cells is a chimeric embryo or a chimeric blastocyst.

2. The population of cells of claim 1, wherein the cell membrane signaling peptide of the one or more cell of a first mammalian species
(a) comprises at least one transmembrane domain;
(b) binds to a signal recognition protein (SRP);
(c) targets the antigen-binding protein to the endoplasmic reticulum (ER) membrane;
(d) introduces a lipid anchor, optionally wherein the lipid anchor is glycosylphosphatidylinositol (GPI); and/or
(e) localizes the antigen-binding protein to the cell membrane.

3. (canceled)

4. The population of cells of claim 1, wherein the membrane signaling peptide comprises an amino acid sequence selected from any one of SEQ ID NOS: 1-4 or 8, or an amino acid sequence having at least 80% identity thereto.

5. The population of cells of claim 1, wherein the cell membrane anchor protein of the one or more cell of the first mammalian species
(a) comprises at least one transmembrane domain;
(b) localizes the antigen-binding protein to cell surface of the cell of a first mammalian species; and/or
(c) is glycosylated or is not glycosylated.

6. The population of cells of claim 1, wherein the cell membrane anchor protein of the one or more cell of the first mammalian species comprises one or more transmembrane domain selected from a CD8 transmembrane domain, ICAM1 transmembrane domain, E-cadherin transmembrane domain, CLDN6 transmembrane domain, ITGB 1 transmembrane domain, ITGB2 transmembrane domain, JAM-B transmembrane domain, NCAM-1 transmembrane domain, MUC-4 transmembrane domain, PDGFRA transmembrane domain, or fragment thereof each, optionally wherein the cell membrane anchor protein of the one or more cell of the first mammalian species is selected from CD8, ICAM1, E-Cad, CLDN6, ITGB1, ITGB2, JAM-B, NCAM-1, MUC-4, PDGFRA, or a fragment thereof each.

7. (canceled)

8. The population of cells of claim 1, wherein the cell membrane anchor protein of the one or more cell of the first mammalian species comprises an amino acid sequence comprising the sequence set forth in any one of SEQ ID NO:9-13, or an amino acid sequence having at least 80% identity thereto.

9. The population of cells of claim 1, wherein the antigen-binding protein of the one or more cell of a first mammalian species comprises an antibody or antigen-binding fragment thereof, optionally wherein the antigen-binding protein comprises a fragment variable domain (FV); a fragment antigen binding (Fab) domain; a Fab′; a (Fab′)2; a half-IgG; a single-chain fragment variable domain (scFv); a diabody (di-scFv); a triabody (tri-scFv)′ a single chain Fab (scFab); a minibody; an scFv-Fc, or a nanobody,
optionally wherein the antigen-binding protein of the one or more cell of a first mammalian species is a nanobody, optionally wherein the nanobody is selected from an anti-GFP nanobody, further optionally wherein the anti-GFP nanobody is selected from vhhGFP4, LaG-2, LaG-3, LaG-5, LaG-6, LaG-8, LaG-9, LaG-10, LaG-11, LaG-12, LaG-17, LaG-18, LaG-43, LaG-19, LaG-21, LaG-42, LaG-24, LaG-26, LaG-27, LaG-29, LaG-30, LaG-35, LaG-37, LaG-41, LaM-2, LaM-3, LaM-4, LaM-6, or LaM-8.

10. (canceled)

11. The population of cells of claim 1, wherein the antigen-binding protein of the one or more cell of a first mammalian species comprises the amino acid sequence set forth in any one of SEQ ID NOS: 9-36, or a sequence having at least 80% identity thereto.

12. The population of cells of claim 1, wherein the antigen-binding protein, cell membrane signaling peptide, and/or a cell membrane anchor protein of the cell of the first mammalian species are connected with linker, optionally wherein the linker comprises the amino acid sequence set forth in any one of SEQ ID NOS: 5-7.

13. The population of cells of claim 1, wherein the antigen of the one or more cell of the second mammalian species is endogenous to the second mammalian species, optionally is endogenous to the first and second mammalian species; or the antigen of the one or more cell of the second mammalian species is exogenous to the second mammalian species, optionally is exogenous to the first and second mammalian species.

14. (canceled)

15. The population of cells of claim 13, wherein the one or more cell of the second mammalian species comprises an exogenous nucleic acid encoding
(a) the exogenous antigen; and
(b) a cell membrane signaling peptide and/or a cell membrane anchor protein, optionally wherein the exogenous antigen is a selected from mCherry, or an isoform thereof; or green-fluorescent protein, or isoform thereof, optionally wherein the exogenous antigen comprises the amino acid sequence set forth in any one of SEQ ID NOS: 14-16, or a sequence having at least 80% identity thereto.

16. (canceled)

17. The population of cells of claim 15, wherein the cell membrane signaling peptide of the one or more cell of a second mammalian species
(a) comprises at least one transmembrane domain;
(b) binds to a signal recognition protein (SRP);
(c) targets the antigen to the endoplasmic reticulum (ER) membrane;
(d) introduces a lipid anchor, optionally wherein the lipid anchor is glycosylphosphatidylinositol (GPI); and/or
(e) localizes the antigen to the cell membrane.

18. (canceled)

19. The population of cells of claim 15, wherein the cell membrane signaling peptide comprises an amino acid sequence selected from any one of SEQ ID NOS: 1-4 or 8, or an amino acid sequence having at least 80% identity thereto.

20. The population of cells of claim 15, wherein the cell membrane anchor protein of the one or more cell of a second mammalian species
(a) comprises at least one transmembrane domain;
(b) localizes the antigen to cell surface of the cell of a second mammalian species; and/or
(c) is glycosylated or is not glycosylated.

21. The population of cells of claim 15, wherein the cell membrane anchor protein of the one or more cell of a second mammalian species comprises one or more transmembrane domain selected from a CD8 transmembrane domain, ICAM1 transmembrane domain, E-cadherin transmembrane domain, CLDN6 transmembrane domain, ITGB1 transmembrane domain, ITGB2 transmembrane domain, JAM-B transmembrane domain, NCAM-1 transmembrane domain, MUC-4 transmembrane domain, PDGFRA transmembrane domain, or fragment thereof each, optionally wherein the cell membrane anchor protein of the one or more cell of the second mammalian species is selected from CD8, ICAM1, E-cad, CLDN6, ITGB1, ITGB2, JAM-B, NCAM-1, MUC-4, PDGFRA, or a fragment thereof each.

22. (canceled)

23. The population of cells of claim 15, wherein the cell membrane anchor protein of the one or more cell of the second mammalian species comprises an amino acid sequence comprising the sequence set forth in any one of SEQ ID NO:9-13, or an amino acid sequence having at least 80% identity thereto.

24. (canceled)

25. The population of cells of claim 1, wherein
(a) one or more cell of the first mammalian species is a human cell and the one or more cell of the second mammalian species a cell of a non-human mammalian species; or
(b) one or more cell of the first mammalian species is a cell of a non-human mammalian species and the one or more cell of the second mammalian species a human cell,
optionally wherein the one or more cell of the non-human mammalian species is selected from: a rodent cell, optionally selected from a mouse cell or rat cell; a non-human primate cell, optionally selected from a monkey cell, chimpanzee cell, gorilla cell, orangutan cell, rhesus macaque cell, marmoset cell or bonobo cell); or an ungulate cell, optionally selected from a pig cell, horse cell, cattle cell, sheep cell, goat cell, or donkey cell,
further optionally wherein the one or more cell of the non-human mammalian species is organogenesis-disabled.

26. (canceled)

27. (canceled)

28. The population of cells of claim 1, wherein the one or more cell of the first mammalian species or the one or more cell of the second mammalian species is a stem cell, optionally an embryonic stem cell,
optionally wherein the one or more cell of the first mammalian species or the one or more cell of the second mammalian species is an induced pluripotent stem cell (iPSC), embryonic stem cell (ESC), or epiblast-derived stem cell (EpiSCs),
further optionally wherein the one or more cell of the first mammalian species is an iPSC and the one or more cell of the second mammalian species is an ESC, or the one or more cell of the first mammalian species is an ESC and the one or more cell of the second mammalian species is an iPSC.

29. (canceled)

30. (canceled)

31. (canceled)

32. (canceled)

33. A method of generating an organ, organoid, or tissue mass comprising of a first mammalian species, the method comprising the steps of:
(a) providing a population of one or more cell of the first mammalian species comprising an antigen-recognition domain at the cell membrane, and providing a population of one or more cell of a second mammalian species comprising cells expressing an antigen at the cell membrane, optionally wherein the first mammalian species is a human, and the second mammalian species is a non-human mammalian; or
providing a population of one or more cell of the first mammalian species comprising an antigen at the cell membrane, and providing a population of one or more cell of a second mammalian species comprising cells expressing an antigen-binding protein at the cell membrane, whereby an interspecies chimera is generated, optionally wherein the interspecies chimera is a chimeric blastocyst,
whereby an interspecies chimera is generated; and
(b) culturing the interspecies chimera to under conditions that allow the interspecies chimera to develop into the organ, organoid, or tissue mass of the cell of a first mammalian species,
optionally wherein the organ, organoid, or tissue mass is a kidney, liver, heart, lung, pancreas, muscle, stomach, intestine, spleen, bladder, reproductive organs, bone marrow, or skin.

34. (canceled)

35. (canceled)

36. (canceled)

37. An organ, organoid, or tissue, which is developed from a population of cells as described herein, or a method as described herein, optionally wherein the organ, organoid, or tissue is a human organ, human organoid, human tissue, and/or comprises more than 90% human cells.

38. (canceled)

39. (canceled)
